(19) United States
(12) Patent Application Publication
(10) Pub. No.: US 2009/0035313 A1
(43) Pub. Date: Feb. 5, 2009

(54) Compounds

(75) Inventors: Christohper Gordon Barber, Kent (GB); Mark Edward Bunnage, Kent (GB); John Wilson Harvey, Kent (GB); John Paul Mathias, Kent (GB)

Correspondence Address: Pfizer Inc.;Corporate Patent Department, P. O. Box 1027, Chesterfield, MO, 63006, US

(73) Assignee: Pfizer Inc.

(21) Appl. No.: 10/564,693

(22) PCT Filed: Jul. 13, 2004
(86) PCT No.: PCT/IB2004/002380

§ 371 Date: Apr. 26, 2007

(30) Foreign Application Priority Data
Jul. 25, 2003 (GB) 0317498.4

(51) Int. Cl. A61K 39/395; C07D 409/12; A61K 31/4436; A61P 11/00; A61K 31/522; A61K 31/56

(52) U.S. Cl. 424/141.1; 546/280.1; 514/336; 514/263.35; 514/171

(57) ABSTRACT

This invention relates to nicotinamide derivatives of general formula (I):

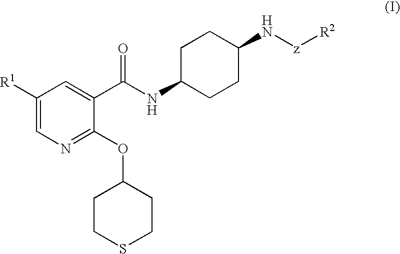

in which R¹, Z and R² have the meanings defined herein, and to processes for the preparation of, intermediates used in the preparation of, compositions containing and the uses of such derivatives.

This application is the National Stage of International Application No. PCT/IB2004/002380, filed Jul. 13, 2004, and claims priority from U.S. patent application Ser. No. 10/896,085 filed Jul. 20, 2004 which claims the benefit of Provisional Application No. 60/497,120 filed Aug. 22, 2003.

This invention relates to nicotinamide derivatives useful as PDE4 inhibitors and to processes for the preparation of, intermediates used in the preparation of, compositions containing and the uses of such derivatives.

The 3′,5′-cyclic nucleotide phosphodiesterases (PDEs) comprise a large class of enzymes divided into at least eleven different families which are structurally, biochemically and pharmacologically distinct from one another. The enzymes within each family are commonly referred to as isoenzymes, or isozymes. A total of more than fifteen gene products is included within this class, and further diversity results from differential splicing and post-translational processing of those gene products. The present invention is primarily concerned with the four gene products of the fourth family of PDEs, i.e., PDE4A, PDE4B, PDE4C, and PDE4D. These enzymes are collectively referred to as being isoforms or subtypes of the PDE4 isozyme family.

The PDE4s are characterized by selective, high affinity hydrolytic degradation of the second messenger cyclic nucleotide, adenosine 3′,5′-cyclic monophosphate (cAMP), and by sensitivity to inhibition by rolipram. A number of selective inhibitors of the PDE4s have been discovered in recent years, and beneficial pharmacological effects resulting from that inhibition have been shown In a variety of disease models (see, e.g., Torphy et al., Environ. Health Perspec., 1994, 102 Suppl. 10, p. 79-84 Duplantier et al., J. Med. Chem., 1996, 39, p. 120-125; Schneider et al., Pharmacol. Biochem. Behav., 1995, 50, p. 211-217; Banner and Page, Br. J. Pharmacol., 1995, 114, p. 93-98; Barnette et al., J. Pharmacol. Exp. Ther., 1995, 273, p. 674-679 Wright et al., Can. J. Physiol. Pharmacol., 1997, 75; p. 1001-1008; Manabe et al., Eur. J. Pharmacol., 1997, 332, p. 97-107 and Ukita et al., J. Med. Chem., 1999, 42, p. 1088-1099). Accordingly, there continues to be considerable interest in the art with regard to the discovery of further selective inhibitors of PDE4s.

Successful results have already been obtained in the art with the discovery and development of selective PDE4 inhibitors. In vivo, PDE4 inhibitors reduce the influx of eosinophils to the lungs of allergen-challenged animals while also reducing the bronchoconstriction and elevated bronchial responsiveness occurring after allergen challenge. PDE4 inhibitors also suppress the activity of Immune cells (including CD4⁺ T-lymphocytes, monocytes, mast cells, and basophils), reduce pulmonary edema, inhibit excitatory nonadrenergic noncholinergic neurotransmission (eNANC), potentiate inhibitory nonadrenergic noncholinergic neurotransmission (iNANC), reduce airway smooth muscle mitogenesis, and induce bronchodilation. PDE4 inhibitors also suppress the activity of a number of inflammatory cells associated with the pathophysiology of COPD, including monocytes/macrophages, CD4⁺ T-lymphocytes, eosinophils and neutrophils. PDE4 inhibitors also reduce vascular smooth muscle mitogenesis and potentially interfere with the ability of airway epithelial cells to generate pro-inflammatory mediators. Through the release of neutral proteases and acid hydrolases from their granules, and the generation of reactive oxygen species, neutrophils contribute to the tissue destruction associated with chronic inflammation, and are further implicated in the pathology of conditions such as emphysema. Therefore, PDE4 inhibitors are particularly useful for the treatment of a great number of inflammatory, respiratory and allergic diseases, disorders or conditions and for wounds and some of them are in clinical development mainly for treatment of asthma, COPD, bronchitis and emphysema.

The effects of PDE4 inhibitors on various inflammatory cell responses can be used as a basis for profiling and selecting inhibitors for further study. These effects include elevation of cAMP and inhibition of superoxide production, degranulation, chemotaxis, and tumor necrosis factor alpha (TNFa) release In eosinophils, neutrophils and monocytes.

Some nicotinamide derivatives having a PDE4 inhibitory activity have already been made. For example, the patent application WO 98/45268 discloses nicotinamide derivatives having activity as selective inhibitors of PDE4D isozyme.

The patent applications WO 01/57036 and WO 03/068235 also disclose nicotinamide derivatives which are PDE4 inhibitors useful in the treatment of various inflammatory allergic and respiratory diseases and conditions.

However, there is still a huge need for additional PDE4 inhibitors that are good drug candidates. In particular, preferred compounds should bind potently to the PDE4 enzyme whilst showing little affinity for other receptors and enzymes. They should also possess favourable pharmacokinetic and metabolic activities, be non-toxic and demonstrate few side effects. Furthermore, it is also desirable that the ideal drug candidate will exist in a physical form that is stable and easily formulated.

The present invention therefore provides new nicotinamide derivatives of formula (I):

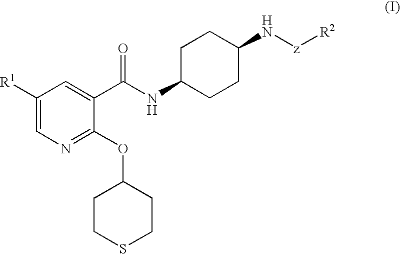

wherein:
R¹ is selected from H, halo and (C₁-C₄)alkyl;
Z is a linker group selected from CO and SO₂;
R² is selected from phenyl, benzyl, naphthyl, heteroaryl and (C₃-C₈)cycloalkyl, each of which being substituted with 1 substituent selected from (C₁-C₆)alkoxy, ((C₃-C₈)cycloalkyl)-(C₁-C₆)alkoxy, hydroxy(C₂-C₆)alkoxy, ((C₃-C₈)cycloalkyl)oxy and phenyl substituted by (C₁-C₆)alkoxy (said phenyl being additionally optionally substituted by OH and/or halo),
and each of which being additionally optionally substituted with 1 or 2 substituents each independently selected from halo, CN, CONR³R⁴, (C₁-C₆)alkyl, halo(C₁-C₆)alkyl, OH, hydroxy(C₁-C₆)alkyl, ((C₃-C₈)cycloalkyl)-(C₁-C₆)alkyl, (C₃-C₈)cycloalkyl and NR³R⁴; and
R³ and R⁴ are each independently selected from H, (C₁-C₄)alkyl, and SO₂(C₁-C₄)alkyl;
and pharmaceutically acceptable salts and solvates thereof.

In the here above general formula (I), halo denotes a halogen atom selected from the group consisting of fluoro (F), chloro (Cl), bromo (Br) and Iodo (I) in particular fluoro or chloro.

(C₁-C₄)alkyl or (C₁-C₆)alkyl or (C₂-C₆)alkyl radicals denote a straight-chain or branched group containing respectively 1 to 4 or 1 to 6 or 2 to 6 carbon atoms. This also applies if they carry substituents or occur as substituents of other radicals, for example in (C₁-C₆)alkoxy radicals, hydroxy(C₁-C₆)alkyl, hydroxy(C₂-C₆)alkoxy radicals and halo(C₁-C₆)alkyl radicals. Examples of suitable (C₁-C₄)alkyl and (C₁-C₆)alkyl radicals are methyl, ethyl, n-propyl, iso-propyl, n-butyl, iso-butyl, sec-butyl, tert-butyl, pentyl, hexyl etc. Examples of suitable (C₁-C₆)alkoxy and (C₂-C₆)alkoxy radicals are methoxy, ethoxy, n-propyloxy, iso-propyloxy, n-butyloxy, iso-butyloxy, sec-butyloxy, tert-butyloxy, pentyloxy, hexyloxy etc. Hydroxy(C₁-C₆)alkyl and hydroxy(C₂-C₆)alkoxy radicals may contain more than one hydroxy group (—OH). According to a preferred embodiment of said invention, such radicals contain one hydroxy substituent. Examples of suitable hydroxy (C₁-C₆)alkyl radicals are hydroxymethyl, 1-hydroxyethyl or 2-hydroxyethyl. Accordingly, halo(C₁-C₆)alkyl radicals may contain more than one halo group. According to a preferred embodiment of said invention, such radicals contain 1, 2 or 3 halo substituent. Examples of suitable halo(C₁-C₆)alkyl radicals are difluoromethyl, trifluoromethyl, difluoroethyl or trifluoroethyl.

In the hereabove general formula (I), “heteroaryl” means a monocyclic or polycyclic ring system comprising at least one aromatic ring, having 5 to 14 ring atoms, which ring system contains 1, 2, 3, 4 or 5 ring heteroatom(s) independently selected from N, O and S. Examples of suitable heteroaryl radicals are pyrrole, furan, furazan, thiophene, imidazole, pyrazole, oxazole, isoxazole, thiazole, isothiazole, tetrazole, triazine, pyridine, pyrazine, pyrimidine, pyridazine, indolizine, indole, isoindole, indazole, purine, naphthyridine, phthalazine, quinoline, isoquinoline, quinoxaline, quinazoline, cinnoline, benzofuran, thiadiazole, benzothiadiazole, oxadiazole, benzofuran, dihydrobenzofuran, benzoxadiazole, benzopyrimidine, benzothiophene, benzoxazole, benzothiazole, imidazopyridine, benzimidazole, pyrazolopyridine, pyrazolopyrimidine, etc. including, where a ring nitrogen atom is present, the corresponding N-oxides and quaternary salts.

Finally, (C₃-C₈)cycloalkyl radical means a 3-membered to 8-membered saturated carbocyclic ring. Examples of suitable (C₃-C₈)cycloalkyl radicals are cyclopropyl, cyclobutyl, cyclopentyl, cyclohexyl, cycloheptyl and cyclooctyl

It has been found that these nicotinamide derivatives are inhibitors of PDE4 isoenzymes, particularly useful for the treatment of inflammatory, respiratory and allergic diseases and conditions or for wounds.

In the general formula (I) according to the present invention, when a radical is mono- or poly-substituted, said substituent(s) can be located at any desired and chemically-feasible position(s). Also, when a radical is polysubstituted, said substituents can be identical or different, unless otherwise stated.

Preferably R¹ is H, halo, CH₃ or C₂H₅. More preferably R¹ is H, F, Cl or CH₃. Most preferably R¹ is F.

Preferably R² is selected from the group consisting of phenyl, imidazole, pyrazine, indazole, purine, quinoline, quinazoline, benzofuran, dihydrobenzofuran, benzothiadiazole, benzoxadiazole, pyrazole, imidazopyridine, benzimidazole, pyrazolopyridine, pyrazolopyrimidine, benzyl and cyclopropyl,

each of which being substituted with 1 substituent selected from (C₁-C₆)alkoxy, ((C₃-C₈)cycloalkyl)(C₁-C₆)alkoxy, hydroxy(C₂-C₆)alkoxy, ((C₃-C₈)cycloalkyl)oxy and phenyl substituted by (C₁-C₆)alkoxy (said phenyl being additionally optionally substituted by OH and/or halo),
and each of which being additionally optionally substituted with 1 or 2 substituents each independently selected from halo, CN, CONR³R⁴, (C₁-C₆)alkyl, halo(C₁-C₆)alkyl, OH, hydroxy(C₁-C₆)alkyl, ((C₃-C₈)cycloalkyl)-(C₁-C₆)alkyl, (C₃-C₈)cycloalkyl and NR³R⁴.

More preferably R² is phenyl, Imidazole, indazole, quinoline, quinazoline, dihydrobenzofuran, benzothiadiazole, benzoxadiazole, pyrazole, imidazopyridine, benzimidazole, pyrazolopyridine, benzyl or cyclopropyl,

each of which being substituted with 1 substituent selected from OCH₃, OC₂H₄OH, O(CH₂)₃OH, OC₂H₅, cyclopropylmethoxy or cyclopentyloxy,
and each of which being additionally optionally substituted by o1 or 2 substituents independently selected from CH₃, N(CH₃)SO₂CH₃, NHSO₂CH₂CH₃, NHSO₂CH(CH₃)₂, OH, CH₂OH, Cl, F, C₂ H₅, CH(CH₃)₂, C₂H₄OH, CF₃.

Most preferably R² is as defined in the Examples.

Preferably Z is CO.

Preferably the compound is selected from any one of the Examples, or a pharmaceutically acceptable salt or solvate thereof.

Preferred compounds according to the present invention are the nicotinamide derivatives of formula (I) wherein:

R¹ is H, halo, CH₃ or C₂H₅, more preferably R¹ is H, F, C₁ or CH₃, and most preferably

### R¹ is F, and

R² is selected from the group consisting of phenyl, imidazole, pyrazine, indazole, purine, quinoline, quinazoline, benzofuran, dihydrobenzofuran, benzothiadiazole, benzoxadiazole, pyrazole, imidazopyridine, benzimidazole, pyrazolopyridine, pyrazolopyrimidine, benzyl and cyclopropyl,
each of which being substituted with 1 substituent selected from (C₁-C₆)alkoxy, ((C₃-C₈)cycloalkyl)-(C₁-C₆)alkoxy, hydroxy(C₂-C₆)alkoxy, ((C₃-C₈)cycloalkyl)oxy and phenyl substituted by (C₁-C₆)alkoxy (said phenyl being additionally optionally substituted by OH and/or halo),
and each of which being additionally optionally substituted with 1 or 2 substituents each independently selected from halo, CN, CONR³R⁴, (C₁-C₆)alkyl, halo(C₁-C₆)alkyl, OH, hydroxy(C₁-C₆)alkyl, ((C₃-C₈)cycloalkyl)-(C₁-C₆)alkyl, (C₃-C₈)cycloalkyl and NR³R⁴.

More preferably, the compounds are selected from the nicotinamide derivatives of formula (I) as described in the here above paragraph wherein Z is CO.

Further preferred compounds according to the present invention are the nicotinamide derivatives of formula (I) wherein:

R¹ is H, halo, CH₃ or C₂H₅, more preferably R¹ is H, F, Cl or CH₃, and most preferably

### R¹ is F, and

R² is phenyl, imidazole, Indazole, quinoline, quinazoline, dihydrobenzofuran, benzothiadiazole, benzoxadiazole, pyrazole, imidazopyridine, benzimidazole, pyrazolopyridine, benzyl or cyclopropyl,
each of which being substituted with 1 substituent selected from OCH₃, OC₂H₄OH, O(CH₂)₃OH, OC₂H₅, cyclopropylmethoxy or cyclopentyloxy, and each of which being additionally optionally substituted by o1or 2 substituents independently selected from CH₃, N(CH₃)SO₂CH₃, NHSO₂CH₂CH₃, NHSO₂CH(CH₃)₂, OH, CH₂OH, Cl, F, C₂H₅, CH(CH₃)₂, C₂H₄OH, CF₃.

Still more preferably, the compounds are selected from the nicotinamide derivatives of formula (I) as described in the here above paragraph wherein Z is CO.

Still more preferably, the compound is selected from any one of Examples 4, 5, 7, 8, 9, 10, 14, 15, 16, 19, 20, 23 and 25 or a pharmaceutically acceptable salt or solvate thereof.

Yet more preferably, the compound is selected from any one of Examples 4, 5, 7, 8, 9, 10, 15 and 20 or a pharmaceutically acceptable salt or solvate thereof.

A most preferred compound is that of Example 5 or a pharmaceutically acceptable salt or solvate thereof.

The nicotinamide derivatives of the formula (I) can be prepared using the Routes disclosed hereunder, and exemplified in the Examples and Preparations, in which the substituents R¹, R² and Z are as previously defined for the nicotinamide derivatives of the formula (I) unless otherwise stated. Other conventional methods may be used in accordance with the skilled person's knowledge.

Unless otherwise provided herein:
PyBOP® means Benzotriazol-1-yloxytris(pyrrolidino)phosphonium hexafluorophosphate;
PyBrOP® means bromo-tris-pyrrolidino-phosphonium hexafluorophosphate;
CDl means N,N′-carbonyldiimidazole;
WSCDI means 1-(3-dimethylaminopropyl)-3-ethylcarbodiimide hydrochloride;
Mukaiyama's reagent means 2-chloro-1-methylpyridinium iodide;
HATU means O-(7-Azabenzotriazol-1-yl)-N,N,N′N′-tetramethyluronium hexafluorophosphate;
HBTU means O-Benzotriazol-1-yl-N,N,N′N′-tetramethyluronium hexafluorophosphate;
DCC means N,N′-dicyclohexylcarbodiimide;
CDI means N,N′-carbonyldiimidazole;
HOAT means 1-hydroxy-7-azabenzotriazole;
HOBT means 1-hydroxybenzotriazole hydrate;
Hünig's base means N-ethyldiisopropylamine;
Et₃N means triethylamine;
NMM means N-methylmorpholine;
NMP means 1-methyl-2-pyrrolidinone;
DMAP means 4-dimethylaminopyridine;
NMO means 4-methylmorpholine N-oxide;
KHMDS means potassium bis(trimethylsilyl)amide;
NaHMDS means sodium bis(trimethylsilyl)amide;
DIAD means diisopropyl azodicarboxylate;
DEAD means diethyl azodicarboxylate;
DIBAL means diisobutylammonium hydride;
Dess-Martin periodinane means 1,1,1-triacetoxy-1,1-dihydro-1,2-benziodoxol-3(1H)-one;
TBDMS-Cl means tert-butyldimethylchlorosilane;
TMS-Cl means chlorotrimethylsilane;
Boc means tert-butoxycarbonyl;
CBz means benzyloxycarbonyl;
MeOH means methanol, EtOH means ethanol, and EtOAc means ethyl acetate;
THF means tetrahydrofuran, DMSO means dimethyl sulphoxide, and DCM means dichloromethane; DMF means N,N-dimethylformamide;
AcOH means acetic acid, TFA means trifluoroacetic acid; rt means room temperature; 3° means tertiary; eq means equivalents; Me means methyl, Et means ethyl, Bn means benzyl; other abbreviations are used in accordance with standard synthetic chemistry practice.

### Route A

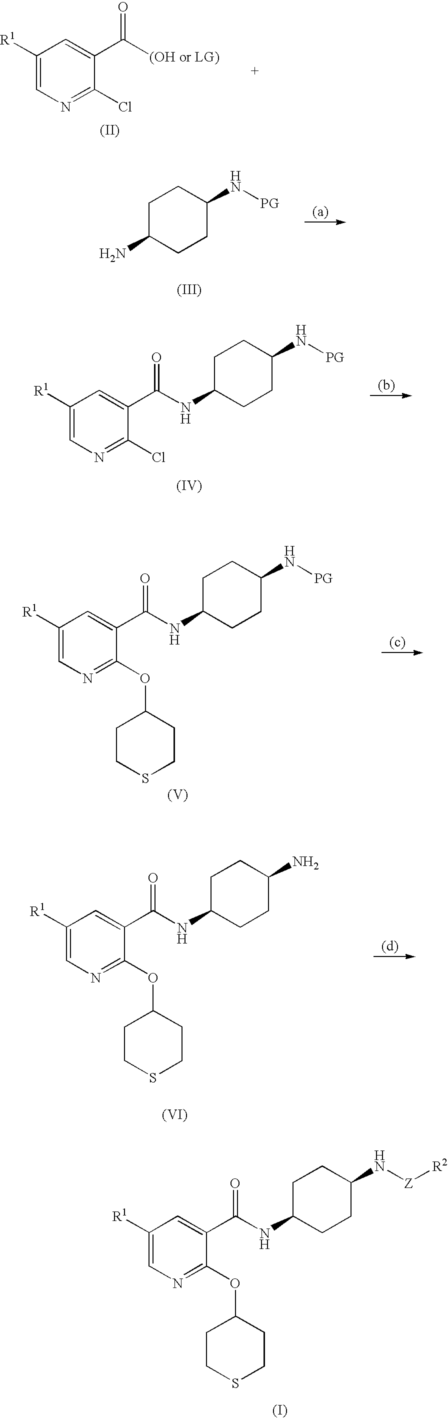

Nicotinic acids of formula (II) are either available commercially or may be obtained by analogy with the methods of Haylor et. al. (EP 0634413); and Marzi et. al. European J. Org. Chem. (2001), (7), 1371-1376.

The protected amines of formula (III) are either available commercially or may be prepared by analogy with the method of Oku et al (WO 99/54284).

In the scheme above, R¹, R² and Z are as previously defined, PG is a suitable amine protecting group, typically Boc, CBz or Bn, and preferably Boc, and LG is a suitable leaving group, typically halo, and preferably Cl.

Step (a)—Acid-Amine Coupling.

This acid/amine coupling may be undertaken by using either

(i) an acyl chloride derivative of acid (II)+amine (III), with an excess of acid acceptor in a suitable solvent, or
(ii) the acid (II) with a conventional coupling agent+amine (III), optionally in the presence of a catalyst, with an excess of acid acceptor in a suitable solvent.

Typically the conditions are as follows:

(i) acid chloride of acid (II) (generated in-situ), an excess of amine (III), optionally with an excess of 3° amine such as Et₃N, Hünig's base or NMM, in DCM or THF, without heating for 1 to 24 hrs, or
(ii) acid (II), WSCDI/DCC/CDI optionally in the presences of HOBT or HOAT, an excess of amine (III), with an excess of NMM, Et₃N, Hünig's base in THF, DCM or EtOAc, at rt. for 4 to 48 hrs; or, acid (II), PYBOP®/PyBrOP®/Mukalyama's reagent/HATU/HBTU, an excess of amine (III), with an excess of NMM, Et₃N, Hünig's base in THF, DCM or EtOAc, at rt. for 4 to 24 hrs.

The preferred conditions are: acid chloride of acid (II) (generated in-situ), about 1.1 eq amine (III), In DCM at rt. for 18 hrs,

Or, acid (II), 1.1 eq amine (III), CDI in DMF at rt. for up to 72 hrs.

Step (b)—Ether Formation

The chloride (IV) is treated with an excess of tetrahydrothiopyran-4-ol, in the presence of a suitable alkali metal base (NaH, K₂CO₃, Cs₂CO₃) in a suitable solvent (eg. MeCN, DMF), optionally in the presence of a catalyst (eg imidazole, DMAP) to provide the ether (V).

The preferred conditions are: chloride (IV), 1.5-2.5 eq tetrahydrothiopyran-4-ol, in the presence of an excess of Cs₂CO₃ in MeCN at about the reflux temperature of the reaction.

### Step (c) —Removal of Protecting Group

Deprotection of the N protecting group (PG) is undertaken using standard methodology, as described in “Protective Groups in Organic Synthesis” by T. W. Greene and P. Wutz.

When PG Is Boc, the preferred conditions are: hydrochloric acid in dioxan and dichloromethane at rt for about 3 hrs.

Step (d) —Reaction of Amino Group with Y-Z-R²

Compounds of the formula (I) may be prepared by reaction of amine (VI) with a suitable reagent of formula Y-Z-R², where Y represents OH or Cl.

When Z represents CO, and Y represents OH or Cl, compounds of formula (I) may be prepared by reaction of the amine of formula (VI) with R²CO₂H according to the general methods described previously for step (a).

The preferred conditions are: WSCDI, HOBT, amine (VI), R²CO₂H, an excess of 3° amine base (Hünig's base, Et₃N or NMM) in dichloromethane, N,N-dimethylformamide, NMP or DMA, at rt. for up to 36 hrs, or amine (VI), acid R²CO₂H, HBTU in the presence of an excess of 3° amine base (Hünig's base, Et₃N or NMM) in DMF for up to 24 hrs at rt.

When Z represents SO₂ and Y represents Cl, compounds of formula (I) may be prepared by reaction of the amine of formula (VI) with R²SO₂Cl by analogy with the general methods described in step (a).

The preferred conditions are: WSCDI, HOBT, amine (VI), R²SO₂Cl, an excess of 3° amine base (Hünig's base, Et₃N or NMM) in N,N-dimethylformamide, at rt. for 18 hrs, or amine (IV), R²SO₂Cl in the presence of excess Et₃N in dichloromethane at rt.

Compounds of formula R²ZY, are either commercially available, or may be obtained using standard methodology, or when R² is a heterocycle, by analogy with the methods described in Comprehensive Heterocyclic Chemistry I and II (Elsevier Science Ltd.) and references therein.

### Step (d) is Exemplified Below in Examples 1-3, 6-13 and 25-29.

### Route B

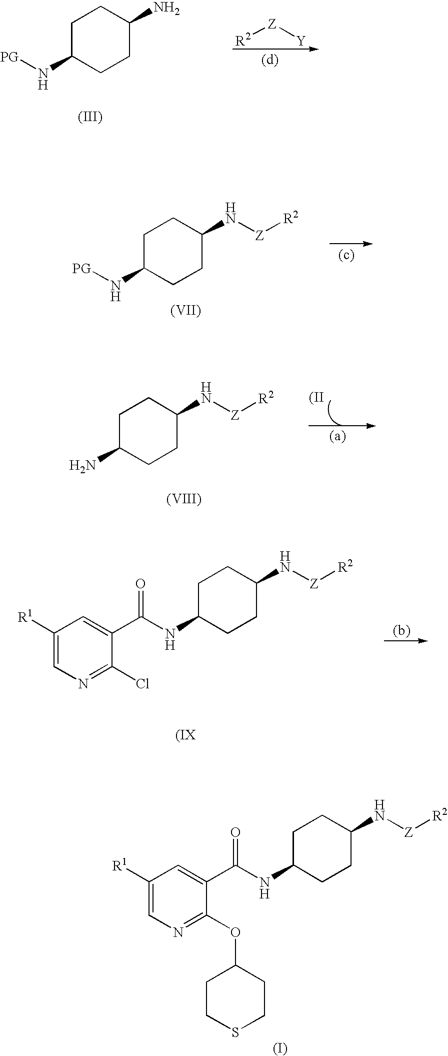

The compound of formula (VII) may be prepared from the amine (III) by reaction with R²ZY according to the methods described previously in step (d), Route A.

The compound of formula (VIII) may be prepared from the compound of formula (VII) by analogy to the methods described previously in step (c), Route A.

Compounds of formula (IX) may be prepared by reaction of the amine of formula (VIII) with the acid or acid derivative (II) according to the methods described previously in step (a), Route A.

Compounds of formula (I) may be prepared by reaction of compounds of formula (IX) with tetrahydrothipyran-4-ol as described previously in step (b), Route A.

The transformation (IX) to (I) is exemplified by Example 88.

### Route C

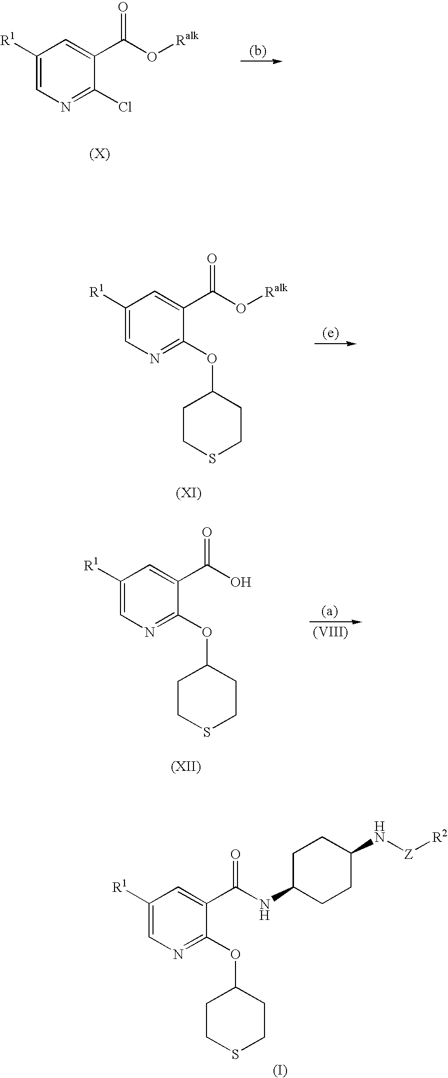

R^{alk} represents a C₁-C₄ alkyl group, preferably Me or Et.

Compounds of formula (X) are either available commercially or may be obtained from the compounds of formula (II), using standard esterification conditions.

Compounds of formula (XI) may be prepared by reaction of the ester (X) with tetrahydrothiopyran-4ol, as described previously in step (b), Route A.

Step (e)—Ester Hydrolysis

Hydrolysis of the ester (XI) may be achieved in the presence of acid or base, in a suitable solvent, optionally at elevated temperature to afford the acid (XII).

Typically, the ester (XI) is treated with an alkali metal hydroxide (eg Li, Na, Cs) in aqueous solvent (MeOH, EtOH, dioxan, THF) at between rt and the reflux temperature of the reaction, to give the acid of formula (XII)

Reaction of the acid (XII) with the amine (VIII) as described previously in step (a) provides the compounds of formula (I).

### Further Routes

Certain R² groups may undergo further functional group interconversions (FGIs) and transformations, such as alkylation of a phenol hydroxy group, using a suitable alkylbromide, In the presence of a suitable alkali metal base (such as K₂CO₃), optionally in the presence of a catalyst (eg KI) in a suitable solvent such as acetonitrile and/or N,N-dimethylformamide at elevated temperature (see ex 15-21), or demethylation of a methoxy group by treatment with lithium iodide in pyridine or collidine, or by treatment with BBr₃ in dichloromethane.

For certain compounds of the description, a suitable protecting group strategy may be employed. For example, a hydroxyl group may be protected using a tetrahydropyran group, and deprotection may be achieved by treatment with a solution of acetic acid:water:tetrahydrofuran (4:1:2 by volume) at rt. for upto 18 hrs. (see e.g. Examples 4 to 21). Further, a benzyloxy group may be used and deprotected to give the corresponding hydroxyl compound, for example by using a reduction (e.g. with palladium black in acid).

FGI and protection/deprotection strategies are exemplified in Examples 4-5 and 22-24.

All of the above reactions and the preparations of novel starting materials used in the preceding methods are conventional and appropriate reagents and reaction conditions for their performance or preparation as well as procedures for isolating the desired products will be well-known to those skilled in the art with reference to literature precedents and the examples and preparations hereto.

As mentioned above, use of protection/deprotection strategies are needed in some instances. Methods such as those described by T. W. GREENE (Protective Groups in Organic Synthesis, A. Wiley-Interscience Publication, 1981) or by McOMIE (Protective Groups in Organic Chemistry, Plenum Press, 1973), can be used.

Compounds of formula (I), as well as Intermediate for the preparation thereof can be purified according to various well-known methods, such as for example crystallization or chromatography.

The nicotinamide derivatives of formula (I) may also be optionally transformed in pharmaceutically acceptable salts. In particular, these pharmaceutically acceptable salts of the nicotinamide derivatives of the formula (I) include the acid addition and the base salts (including disalts) thereof.

Suitable acid addition salts are formed from acids which form non-toxic salts. Examples include the acetate, aspartate, benzoate, besylate, bicarbonate/carbonate, bisulphate, camsylate, citrate, edisylate, esylate, fumarate, gluceptate, gluconate, glucuronate, hibenzate, hydrochloride/chloride, hydrobromide/bromide, hydroiodide/iodie, hydrogen phosphate, isethionate, D- and L-lactate, malate, maleate, malonate, mesylate, methylsulphate, 2-napsylate, nicotinate, nitrate, orotate, palmoate, phosphate, saccharate, stearate, succinate sulphate, D- and L-tartrate, 1-hydroxy-2-naphtoate, 3-hydroxy-2-naphthoate and tosylate saltes.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see Stahl and Wermuth, Handbook of Pharmaceutical Salts: Properties, Selection and Use, Wiley-VCH, Weinheim, Germany (2002).

A pharmaceutically acceptable salt of a nicotinamide derivative of the formula (I) may be readily prepared by mixing together solutions of the nicotinamide derivative of formula (I) and the desired acid or base, as appropriate. The salt may precipitate from solution and be collected by filtration or may be recovered by evaporation of the solvent.

Pharmaceutically acceptable solvates in accordance with the invention include hydrates and solvates wherein the solvent of crystallization may be isotopically substituted, e.g. D₂O, d₆-acetone, d₆-DMSO.

Also within the scope of the invention are clathrates, drug-host inclusion complexes wherein, in contrast to the aforementioned solvates, the drug and host are present in non-stoichlometric amounts. For a review of such complexes, see J Pharm Sci, 64 (8), 1269-1288 by Haleblian (August 1975).

Hereinafter all references to nicotinamide derivatives of formula (I) include references to salts thereof and to solvates and clathrates of compounds of formula (I) and salts thereof.

The invention includes all polymorphs of the nicotinamide derivatives of formula (I).

Also within the scope of the invention are so-called “prodrugs” of the nicotinamide derivatives of formula (I). Thus certain derivatives of nicotinamide derivatives of formula (I) which have little or no pharmacological activity themselves can, when metabolised upon administration into or onto the body, give rise to nicotinamide derivatives of formula (I) having the desired activity. Such derivatives are referred to as “prodrugs”.

Prodrugs in accordance with the invention can, for example, be produced by replacing appropriate functionalities present in the nicotinamide derivatives of formula (I) with certain moieties known to those skilled in the art as “pro-moieties” as described, for example, in “Design of Prodrugs” by H Bundgaard (Elsevier, 1985).

Finally, certain nicotinamide derivatives of formula (I) may themselves act as prodrugs of other nicotinamide derivatives of formula (I).

Nicotinamide derivatives of formula (I) containing one or more asymmetric carbon atoms can exist as two or more optical isomers. Where a nicotinamide derivative of formula (I) contains an alkenyl or alkenylene group, geometric cis/trans (or Z/E) isomers are possible, and where the nicotinamide derivative contains, for example, a keto or oxime group, tautomeric isomerism (‘tautomerism’) may occur. It follows that, unless otherwise defined, a single nicotinamide derivative may exhibit more than one type of isomerism.

Included within the scope of the present invention are all optical isomers, geometric isomers and tautomeric forms of the nicotinamide derivatives of formula (I), including compounds exhibiting more than one type of isomerism, and mixtures of one or more thereof.

Cis/trans isomers may be separated by conventional techniques well known to those skilled in the art, for example, fractional crystallisation and chromatography.

Conventional techniques for the preparation/isolation of individual stereoisomers include the conversion of a suitable optically pure precursor, resolution of the racemate (or the racemate of a salt or derivative) using, for example, chiral HPLC, or fractional crystallisation of diastereoisomeric salts formed by reaction of the racemate with a suitable optically active acid or base, for example, tartaric acid.

The present invention also includes all pharmaceutically acceptable isotopic variations of a nicotinamide derivative of formula (I). An isotopic variation is defined as one in which at least one atom is replaced by an atom having the same atomic number, but an atomic mass different from the atomic mass usually found in nature.

Examples of isotopes suitable for inclusion in the nicotnamide derivatives of the invention include isotopes of hydrogen, such as ²H and ³H, carbon such as ¹³C and ¹⁴C, nitrogen, such as ¹⁵N, oxygen, such as ¹⁷O and ¹⁸O, phosphorus, such as ³²P, sulphur, such as ³⁵S, fluorine, such as ¹⁸F, and chlorine, such as ³⁶Cl.

Substitution of the nicotinamide derivative of formula (I) isotopes such as deuterium, i.e. ²H, may afford certain therapeutic advantages resulting from greater metabolic stability, for example, increased in vivo half-life or reduced dosage requirements, and hence may be preferred in some circumstances.

Certain isotopic variations of the nicotinamide derivatives of formula (I), for example, those incorporating a radioactive isotope, are useful in drug and/or substrate tissue distribution studies. The radioactive Isotopes tritium, i.e. ³H, and carbon-14, i.e. ¹⁴C, are particularly useful for this purpose in view of their ease of incorporation and ready means of detection.

Isotopic variations of the nicotinamide derivatives of formula (I) can generally be prepared by conventional techniques known to those skilled in the art or by processes analogous to those described in the accompanying Examples and Preparations using appropriate isotopic variations of suitable reagents.

According to a further aspect, the present invention concerns mixtures of nicotinamide derivatives of the formula (I), as well as mixtures with or of their pharmaceutically acceptable salts, solvates, polymorphs, isomeric forms and/or isotope forms.

According to the present invention, all the here above mentioned forms of the nicotinamide derivatives of formula (I) except the pharmaceutically acceptable salts (i.e. said solvates, polymorphs, isomeric forms and isotope forms), are defined as “derived forms” of the nicotinamide derivatives of formula (I) in what follows.

The nicotinamide derivatives of formula (I), their pharmaceutically acceptable salts and/or derived forms, are valuable pharmaceutical active compounds, which are suitable for the therapy and prophylaxis of numerous disorders in which the PDE4 enzymes are involved, in particular the inflammatory disorders, allergic disorders, respiratory diseases and wounds.

The nicotinamide derivatives of formula (I) and their pharmaceutically acceptable salts and derived forms as mentioned above can be administered according to the invention to animals, preferably to mammals, and in particular to humans, as pharmaceuticals for therapy or prophylaxis. They can be administered per se, in mixtures with one another or in combination with other drugs, or in the form of pharmaceutical preparations which permit enteral (gastric) or parenteral (non-gastric) administration and which as active constituent contain an efficacious dose of at least one nicotinamide derivative of the formula (I), its pharmaceutically acceptable salts and/or derived forms, in addition to customary pharmaceutically Innocuous excipients and/or additives. The term “excipient” Is used herein to describe any ingredient other than the compound of the invention. The choice of excipient will to a large extent depend on the particular mode of administration.

The nicotinamide derivatives of formula (I), their pharmaceutically acceptable salts and/or derived forms may be freeze-dried, spray-dried, or evaporatively dried to provide a solid plug, powder, or film of crystalline or amorphous material. Microwave or radio frequency drying may be used for this purpose.

### Oral Administration

The nicotinamide derivatives of formula (I) their pharmaceutically acceptable salts and/or derived forms of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, propylene glycol, methylcellulose, or a suitable oil, and one or more emulsifying agents and/or suspending agents. Liquid formulations may also be prepared by the reconstitution of a solid, for example, from a sachet.

The nicotinamide derivatives of formula (I), their pharmaceutically acceptable salts and/or derived forms of the invention may also be used in fast-dissolving, fast-disintegrating dosage forms such as those described In Expert Opinion in Therapeutic Patents, 11 (6), 981-986 by Liang and Chen (2001).

The composition of a typical tablet in accordance with the invention may comprise:

| Ingredient | % w/w |
|---|---|
| Nicotinamide derivative of formula (I) | 10.00* |
| Microcrystalline cellulose | 64.12 |
| Lactose | 21.38 |
| Croscarmellose sodium | 3.00 |
| Magnesium stearate | 1.50 |

*Quantity adjusted in accordance with drug activity.

A typical tablet may be prepared using standard processes known to a formulation chemist, for example, by direct compression, granulation (dry, wet, or melt), melt congealing, or extrusion. The tablet formulation may comprise one or more layers and may be coated or uncoated.

Examples of excipients suitable for oral administration include carriers, for example, cellulose, calcium carbonate, dibasic calcium phosphate, mannitol and sodium citrate, granulation binders, for example, polyvinylpyrrolidine, hydroxypropylcellulose, hydroxypropylmethylcellulose and gelatin, disintegrants, for example, sodium starch glycolate and silicates, lubricating agents, for example, magnesium stearate and stearic acid, wetting agents, for example, sodium lauryl sulphate, preservatives, anti-oxidants, flavours and colourants.

Solid formulations for oral administration may be formulated to be Immediate and/or modified release. Modified release formulations include delayed-, sustained-, pulsed-, controlled dual-, targeted and programmed release. Details of suitable modified release technologies such as high energy dispersions, osmotic and coated particles are to be found in Verma et al, Pharmaceutical Technology On-line, 25(2), 1-14 (2001). Other modified release formulations are described in U.S. Pat. No. 6,106,864.

### Parenteral Administration

The nicotinamide derivatives of formula (I), their pharmaceutically acceptable salts and/or derived forms of the invention may also be administered directly Into the blood stream, into muscle, or into an internal organ. Suitable means for parenteral administration include Intravenous, intraarterial, Intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of nicotinamide derivatives of formula (I) used in the preparation of parenteral solutions may be increased by suitable processing, for example, the use of high energy spray-dried dispersions (see WO 01/47495) and/or by the use of appropriate formulation techniques, such as the use of solubility-enhancing agents.

Formulations for parenteral administration may be formulated to be immediate and/or modified release. Modified release formulations include delayed-, sustained-, pulsed-, controlled dual-, targeted and programmed release.

### Topical Administration

The nicotinamide derivatives of the invention may also be administered topically to the skin or mucosa, either dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin and propylene glycol. Penetration enhancers may be incorporated—see, for example, J Pharm Sci, 88 (10), 955-958 by Finnin and Morgan (October 1999).

Other means of topical administration include delivery by iontophoresis, electroporation, phonophoresis, sonophoresis and needle-free or microneedle injection.

Formulations for topical administration may be formulated to be immediate and/or modified release. Modified release formulations include delayed-, sustained-, pulsed-, controlled dual-, targeted and programmed release. Thus nicotinamide derivatives of formula (I) may be formulated in a more solid form for administration as an implanted depot providing long-term release of the active compound.

### Inhaled/Intranasal Administration

The nicotinamide derivatives of formula (I) can also be administered intranasally or by inhalation, typically in the form of a dry powder (either alone, as a mixture, for example, in a dry blend with lactose in anhydrous or monohydrate form, preferably monohydrate, mannitol, dextran, glucose, maltose, sorbitol, xylitol, fructose, sucrose or trehalose, or as a mixed component particle, for example, mixed with phospholipids) from a dry powder inhaler or as an aerosol spray from a pressurised container, pump, spray, atomiser (preferably an atomiser using electrohydrodynamics to produce a fine mist), or nebuliser, with or without the use of a suitable propellant, such as dichlorofluoromethane.

The pressurised container, pump, spray, atomizer, or nebuliser contains a solution or suspension of the active compound comprising, for example, ethanol (optionally, aqueous ethanol) or a suitable alternative agent for dispersing, solubilising, or extending release of the active, the propellant(s) as solvent and an optional surfactant, such as sorbitan trioleate or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

A suitable solution formulation for use in an atomiser using electrohydrodynamics to produce a fine mist may contain from 1 μg to 20 mg of the nicotinamide derivative of formula (I) per actuation and the actuation volume may vary from 1 μl to 100 μl. A typical formulation may comprise a nicotinamide derivative of formula (I), propylene glycol, sterile water, ethanol and sodium chloride. Alternative solvents which may be used instead of propylene glycol include glycerol and polyethylene glycol.

Capsules, blisters and cartridges (made, for example, from gelatin or HPMC) for use in an inhaler or insufflator may be formulated to contain a powder mix of the nicotinamide derivative of formula (I), a suitable powder base such as lactose or starch and a performance modifier such as I-leucine, mannitol, or magnesium stearate.

In the case of dry powder inhalers and aerosols, the dosage unit is determined by means of a valve which delivers a metered amount. Units in accordance with the invention are typically arranged to administer a metered dose or “puff” containing from 1 μg to 4000 μg of the nicotinamide derivative of formula (I). The overall daily dose will typically be in the range 1 μg to 20 mg which may be administered in a single dose or, more usually, as divided doses throughout the day.

Formulations for inhaled/intranasal administration may be formulated to be immediate and/or modified release. Modified release formulations include delayed-, sustained-, pulsed-, controlled dual-, targeted and programmed release. Sustained or controlled release can be obtained by using for example poly(D,L-lactic-co-glycolic acid).

Flavouring agents, such as methol and levomethol and/or sweeteners such as saccharing or saccharin sodium can be added to the formulation.

According to a preferred aspect, the nicotinamide derivatives of formula (I) of the present invention are administered intranasally or by inhalation.

### Rectal/Intravaginal Administration

The nicotinamide derivatives of formula (I) may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled dual-, targeted and programmed release.

### Ocular/Andial Administration

The nicotinamide derivatives of formula (I) may also be administered directly to the eye or ear, typically in the form of drops of a micronised suspension or solution in isotonic, pH-adjusted, sterile saline. Other formulations suitable for ocular and andial administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/andial administration may be formulated to be Immediate and/or modified release. Modified release formulations include delayed-, sustained-, pulsed-, controlled dual-, targeted, or programmed release.

### Enabling Technologies

The nicotinamide derivatives of formula (I) may be combined with soluble macromolecular entities such as cyclodextrin or polyethylene glycol-containing polymers to improve their solubility, dissolution rate, taste-masking, bioavailability and/or stability.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

### Dosage

For administration to human patients, the total daily dose of the nicotinamide derivatives of formula (I) is typically In the range 0.001 mg/kg to 100 mg/kg depending, of course, on the mode of administration. The total daily dose may be administered in single or divided doses. The physician will readily be able to determine doses for subjects depending on age, weight, health state and sex or the patient as well as the severity of the disease.

According to another embodiment of the present invention, the nicotinamide derivatives of the formula (I), their pharmaceutically acceptable salts and/or their derived forms, can also be used as a combination with one or more additional therapeutic agents to be co-administered to a patient to obtain some particularly desired therapeutic end result. The second and more additional therapeutic agents may also be a nicotinamide derivatives of the formula (I), their pharmaceutically acceptable salts and/or their derived forms, or one or more PDE4 inhibitors known in the art. More typically, the second and more therapeutic agents will be selected from a different class of therapeutic agents.

As used herein, the terms “co-administration”, “co-administered” and “in combination with”, referring to the nicotinamide derivatives of formula (I) and one or more other therapeutic agents, is intended to mean, and does refer to and include the following:
simultaneous administration of such combination of nicotinamide derivative(s) and therapeutic agent(s) to a patient in need of treatment, when such components are formulated together into a single dosage form which releases said components at substantially the same time to said patient,
substantially simultaneous administration of such combination of nicotinamide derivative(s) and therapeutic agent(s) to a patient in need of treatment, when such components are formulated apart from each other into separate dosage forms which are taken at substantially the same time by said patient, whereupon said components are released at substantially the same time to said patient,
sequential administration of such combination of nicotinamide derivative(s) and therapeutic agent(s) to a patient in need of treatment, when such components are formulated apart from each other Into separate dosage forms which are taken at consecutive times by said patient with a significant time interval between each administration, whereupon said components are released at substantially different times to said patient; and
sequential administration of such combination of nicotinamide derivative(s) and therapeutic agent(s) to a patient in need of treatment, when such components are formulated together into a single dosage form which releases said components in a controlled manner whereupon they are concurrently, consecutively, and/or overlappingly administered at the same and/or different times by said patient.

Suitable examples of other therapeutic agents which may be used in combination with the nicotinamide derivatives of the formula (I), their pharmaceutically acceptable salts and/or their derived forms include, but are by no mean limited to:

(a) 5-Lipoxygenase (5-LO) inhibitors or 5-lipoxygenase activating protein (FLAP) antagonists,
(b) Leukotriene antagonists (LTRAs) including antagonists of LTB4, LTC4, LTD4, and LTE4,
(c) Histaminic receptor antagonists including H1, H3 and H4 antagonists,
(d) α1- and α2-adrenoceptor agonist vasoconstrictor sympathomimetic agents for decongestant use,
(e) Muscarinic M3 receptor antagonists or anticholinergic agents,
(f) β2-adrenoceptor agonists,

### (g) Theophylline,

(h) Sodium cromoglycate,
(i) COX-1 inhibitors (NSAIDs) and COX-2 selective inhibitors,
(j) Oral or inhaled Glucocorticosteroids,
(k) Monoclonal antibodies active against endogenous inflammatory entities,
(l) Anti-tumor necrosis factor (anti-TNF-a) agents,
(m) Adhesion molecule inhibitors including VLA-4antagonists,
(n) Kinin-B1- and B2-receptor antagonists,
(o) Immunosuppressive agents,
(p) Inhibitors of matrix metalloproteases (MMPs),
(q) Tachykinin NK1, NK2 and NK3 receptor antagonists,
(r) Elastase inhibitors,
(s) Adenosine A2a receptor agonists,
(t) Inhibitors of urokinase,
(u) Compounds that act on dopamine receptors, e.g. D2 agonists,
(v) Modulators of the NFkb pathway, e.g. IKK inhibitors,
(w) Agents that can be classed as mucolytics or anti-tussive,
(x) antibiotics, and
(y) p38 MAP kinase inhibitors

According to the present invention, combination of the nicotinamide derivatives of formula (I) with:
muscarinic M3 receptor agonists or anticholinergic agents including in particular ipratropium salts, namely bromide, tiotropium salts, namely bromide, oxitropium salts, namely bromide, perenzepine, and telenzepine,
β2-adrenoceptor agonists including albutarol, salbutamol, formoterol and salmeterol,
p38 MAP kinase inhibitors,
H3 anatgonists,
glucocorticosteroids, in particular inhaled glucocorticosteroids with reduced systemic side effects, including prednisone, prednisolone, flunisolide, triamcinolone acetonide, beclomethasone dipropionate, budesonide, fluticasone propionate, and mometasone furoate,
or adenosine A2a receptor agonists,
   are preferred.

It is to be appreciated that all references herein to treatment include curative, palliative and prophylactic treatment. The description which follows concerns the therapeutic applications to which the nicotinamide derivatives of formula (I) may be put.

The nicotinamide derivatives of formula (I) inhibit the PDE4 Isozyme and thereby have a wide range of therapeutic applications, as described further below, because of the essential role, which the PDE4 family of isozymes plays in the physiology of all mammals. The enzymatic role performed by the PDE4 isozymes is the intracellular hydrolysis of adenosine 3′,5′-monophosphate (cAMP) within pro-inflammatory leukocytes, cAMP, in turn, is responsible for mediating the effects of numerous hormones in the body, and as a consequence, PDE4 inhibition plays a significant role in a variety of physiological processes. There is extensive literature in the art describing the effects of PDE inhibitors on various inflammatory cell responses, which in addition to cAMP increase, include inhibition of superoxide production, degranulation, chemotaxis and tumor necrosis factor (TNF) release in eosinophils, neutrophils and monocytes.

Therefore, a further aspect of the present invention relates to the use of the nicotinamide derivatives of formula (I), their pharmaceutically acceptable salts and/or derived forms, In the treatment of diseases, disorders, and conditions in which the PDE4 isozymes are involved. More specifically, the present invention also concerns the use of the nicotinamide derivatives of formula (I), their pharmaceutically acceptable salts and/or derived forms, in the treatment of diseases, disorders, and conditions selected from the group consisting of:
asthma of whatever type, etiology, or pathogenesis, in particular asthma that is a member selected from the group consisting of atopic asthma, non-atopic asthma, allergic asthma, atopic bronchial IgE-mediated asthma, bronchial asthma, essential asthma; true asthma, intrinsic asthma caused by pathophysiologic disturbances, extrinsic asthma caused by environmental factors, essential asthma of unknown or inapparent cause, non-atopic asthma, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma and wheezy infant syndrome,
chronic or acute bronchoconstriction, chronic bronchitis, small airways obstruction, and emphysema,
obstructive or inflammatory airways diseases of whatever type, etiology, or pathogenesis, in particular an obstructive or inflammatory airways disease that is a member selected from the group consisting of chronic eosinophilic pneumonia, chronic obstructive pulmonary disease (COPD), COPD that includes chronic bronchitis, pulmonary emphysema or dyspnea associated therewith, COPD that is characterized by irreversible, progressive airways obstruction, adult respiratory distress syndrome (ARDS) and exacerbation of airways hyper-reactivity consequent to other drug therapy
pneumoconiosis of whatever type, etiology, or pathogenesis, in particular pneumoconiosis that is a member selected from the group consisting of aluminosis or bauxite workers' disease, anthracosis or miners' asthma, asbestosis or steam-fitters' asthma, chalicosis or flint disease, ptilosis caused by inhaling the dust from ostrich feathers, siderosis caused by the inhalation of iron particles, silicosis or grinders' disease, byssinosis or cotton-dust asthma and talc pneumoconiosis;
bronchitis of whatever type, etiology, or pathogenesis, in particular bronchitis that is a member selected from the group consisting of acute bronchitis, acute laryngotracheal bronchitis, arachidic bronchitis, catarrhal bronchitis, croupus bronchitis, dry bronchitis, infectious asthmatic bronchitis, productive bronchitis, staphylococcus or streptococcal bronchitis and vesicular bronchitis,
bronchiectasis of whatever type, etiology, or pathogenesis, in particular bronchiectasis that is a member selected from the group consisting of cylindric bronchiectasis, sacculated bronchiectasis, fusiform bronchiectasis, capillary bronchiectasis, cystic bronchiectasis, dry bronchiectasis and follicular bronchiectasis,
seasonal allergic rhinitis or perennial allergic rhinitis or sinusitis of whatever type, etiology, or pathogenesis, in particular sinusitis that is a member selected from the group consisting of purulent or nonpurulent sinusitis, acute or chronic sinusitis and ethmoid, frontal, maxillary, or sphenoid sinusitis,
rheumatoid arthritis of whatever type, etiology, or pathogenesis, in particular rheumatoid arthritis that is a member selected from the group consisting of acute arthritis, acute gouty arthritis, chronic inflammatory arthritis, degenerative arthritis, infectious arthritis, Lyme arthritis, proliferative arthritis, psoriatic arthritis and vertebral arthritis,
gout, and fever and pain associated with inflammation,
an eosinophil-related disorder of whatever type, etiology, or pathogenesis, in particular an eosinophil-related disorder that is a member selected from the group consisting of eosinophilia, pulmonary infiltration eosinophilia, Loffler's syndrome, chronic eosinophilic pneumonia, tropical pulmonary eosinophilia, bronchopneumonic aspergillosis, aspergilloma, granulomas containing eosinophils, allergic granulomatous angiitis or Churg-Strauss syndrome, polyarteritis nodosa (PAN) and systemic necrofizing vasculitis,
atopic dermatitis, allergic dermatitis, contact dermatitis, or allergic or atopic eczema,
urticaria of whatever type, etiology, or pathogenesis, in particular urticaria that is a member selected from the group consisting of immune-mediated urticaria, complement-mediated urticaria, urticariogenic material-induced urticaria, physical agent-induced urticaria, stress-induced urticaria, idiopathic urticaria, acute urticaria, chronic urticaria, angioedema, cholinergic urticaria, cold urticaria in the autosomal dominant form or in the acquired form, contact urticaria, giant urticaria and papular urticaria,
conjunctivitis of whatever type, etiology, or pathogenesis, in particular conjunctivitis that is a member selected from the group consisting of actinic conjunctivitis, acute catarrhal conjunctivitis, acute contagious conjunctivitis, allergic conjunctivitis, atopic conjunctivitis, chronic catarrhal conjunctivitis, purulent conjunctivitis and vernal conjunctivitis,
uveitis of whatever type, etiology, or pathogenesis, in particular uveitis that is a member selected from the group consisting of inflammation of all or part of the uvea, anterior uveitis, iritis, cyclitis, iridocyclitis, granulomatous uveitis, nongranulomatous uveitis, phacoantigenic uveitis, posterior uveitis, choroiditis; and chorioretinitis,
psoriasis;
multiple sclerosis of whatever type, etiology, or pathogenesis, in particular multiple sclerosis that is a member selected from the group consisting of primary progressive multiple sclerosis and relapsing remitting multiple sclerosis,
autoimmune/inflammatory diseases of whatever type, etiology, or pathogenesis, in particular an autoimmune/inflammatory disease that is a member selected from the group consisting of autoimmune hematological disorders, hemolytic anemia, aplastic anemia, pure red cell anemia, idiopathic thrombocytopenic purpura, systemic lupus erythematosus, polychondritis, scleroderma, Wegner's granulomatosis, dermatomyositis, chronic active, hepatitis, myasthenia gravis, Stevens-Johnson syndrome, idiopathic sprue, autoimmune inflammatory bowel diseases, ulcerative colitis, endocrin opthamopathy, Grave's disease, sarcoidosis, alveolitis, chronic hypersensitivity pneumonitis, primary biliary cirrhosis, juvenile diabetes or diabetes mellitus type I, keratoconjunctivitis sicca, epidemic keratoconjunctivitis, diffuse interstitial pulmonary fibrosis or interstitial lung fibrosis, idiopathic pulmonary fibrosis, cystic fibrosis, glomerulonephritis with and without nephrotic syndrome, acute glomerulonephritis, idiopathic nephrotic syndrome, minimal change nephropathy, inflammatory/hyperproliferative skin diseases, benign familial pemphigus, pemphigus erythematosus, pemphigus foliaceus, and pemphigus vulgaris,
prevention of allogeneic graft rejection following organ transplantation,
inflammatory bowel disease (IBD) of whatever type, etiology, or pathogenesis, in particular Inflammatory bowel disease that is a member selected from the group consisting of collagenous colitis, colitis polyposa, transmural colitis, ulcerative colitis and Crohn's disease (CD),
septic shock of whatever type, etiology, or pathogenesis, in particular septic shock that is a member selected from the group consisting of renal failure, acute renal failure, cachexia, malarial cachexia, hypophysial cachexia, uremic cachexia, cardiac cachexia, cachexia suprarenalis or Addison's disease, cancerous cachexia and cachexia as a consequence of infection by the human immunodeficiency virus (HIV),
liver injury,
pulmonary hypertension of whatever type, etiology or pathogenesis including primary pulmonary hypertension/essential hypertension, pulmonary hypertension secondary to congestive heart failure, pulmonary hypertension secondary to chronic obstructive pulmonary disease, pulmonary venous hypertension, pulmonary arterial hypertension and hypoxia-induced pulmonary hypertension,
bone loss diseases, primary osteoporosis and secondary osteoporosis,
central nervous system disorders of whatever type, etiology, or pathogenesis, in particular a central nervous system disorder that is a member selected from the group consisting of depression, Alzheimers disease, Parkinson's disease, learning and memory impairment, tardive dyskinesia, drug dependence, arteriosclerotic dementia and dementias that accompany Huntington's chorea, Wilson's disease, paralysis agitans, and thalamic atrophies,
infection, especially infection by viruses wherein such viruses increase the production of TNF-α in their host, or wherein such viruses are sensitive to upregulation of TNF-α in their host so that their replication or other vital activities are adversely impacted, including a virus which is a member selected from the group consisting of HIV-1, HIV-2, and HIV-3, cytomegalovirus (CMV), Influenza, adenoviruses and Herpes viruses including Herpes zoster and Herpes simplex,
yeast and fungus infections wherein said yeast and fungi are sensitive to upregulation by TNF-α or elicit TNF-α production in their host, e.g., fungal meningitis, particularly when administered in conjunction with other drugs of choice for the treatment of systemic yeast and fungus infections, including but are not limited to, polymixins, e.g. Polymycin B, imidazoles, e.g. clotrimazole, econazole, miconazole, and ketoconazole, triazoles, e.g. fluconazole and itranazole as well as amphotericins, e.g. Amphotericin B and liposomal Amphotericin B,
ischemia-reperfusion injury, Ischemic heart disease, autoimmune diabetes, retinal autoimmunity, chronic lymphocytic leukemia, HIV infections, lupus erythematosus, kidney and ureter disease, urogenital and gastrointestinal disorders and prostate diseases,
reduction of scar formation in the human or animal body, such as scar formation in the healing of acute wounds, and
psoriasis, other dermatological and cosmetic uses, including antiphlogistic, skin-softening, skin elasticity and moisture-increasing activities.

According to one aspect the present invention relates in particular to the treatment of a respiratory disease, such as adult respiratory distress syndrome (ARDS), bronchitis, chronic bronchitis, chronic obstructive pulmonary disease (COPD), cystic fibrosis, asthma, emphysema, bronchiectasis, chronic sinusitis and rhinitis.

According to another aspect the present invention relates In particular to the treatment of gastrointestinal (GI) disorders, In particular inflammatory bowel diseases (IBD) such as Crohn's disease, ileitis, collagenous colitis, colitis polyposa, transmural colitis and ulcerative colitis.

A still further aspect of the present invention also relates to the use of the nicotinamide derivatives of formula (I), their pharmaceutically acceptable salts and/or derived forms, for the manufacture of a drug having a PDE4 inhibitory activity. In particular, the present inventions concerns the use of the nicotinamide derivatives of formula (I), their pharmaceutically acceptable salts and/or derived forms, for the manufacture of a drug for the treatment of inflammatory, respiratory, allergic and scar-forming diseases, disorders, and conditions, and more precisely for the treatment of diseases, disorders, and conditions that are listed above.

As a consequence, the present invention provides a particularly interesting method of treatment of a mammal, including a human being, with a PDE4 inhibitor including treating said mammal with an effective amount of a nicotinamide derivative of formula (I), its pharmaceutically acceptable salts and/or derived forms. More precisely, the present invention provides a particularly interesting method of treatment of a mammal, including a human being, to treat an inflammatory, respiratory, allergic and scar-forming disease, disorder or condition, including treating said mammal with an effective amount of a nicotinamide derivative of formula (I), its pharmaceutically acceptable salts and/or derived forms.

Further aspects of the invention are mentioned in the claims.

The following Examples illustrate the preparation of the nicotinamide derivatives of the formula (I):

### EXAMPLE 1

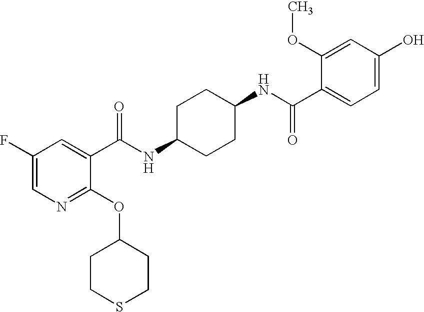

The amine hydrochloride from preparation 15a was dissolved in dichloromethane, the solution washed with 1N sodium hydroxide solution, then dried (MgSO₄) and evaporated under reduced pressure. 1-(3-Dimethylaminopropyl)-3-ethylcarbodiimide hydrochloride (200 mg, 1.05 mmol) was added to a solution of the freshly prepared amine (200 mg, 0.57 mmol), 1-hydroxybenzotriazole hydrate (93 mg, 0.69 mmol), the appropriate acid (0.52 mmol) and N-ethyldiisopropylamine (480 μl, 2.28 mmol) in N,N-dimethylformamide (3 ml), and the reaction stirred at room temperature for 18 hours. The mixture was partitioned between ethyl acetate and 2N hydrochloric acid and the layers separated. The organic phase was washed with additional 2N hydrochloric acid, sodium bicarbonate solution, water and brine, then dried (MgSO₄) and concentrated under reduced pressure. The crude products were purified by column chromatography on silica gel using an elution gradient of ethyl acetate:pentane (30:70 to 100:0) or using acetonitrile:dichloromethane (1:99 to 50:50). The products were then azeotroped with dichloromethane:diisopropyl ether and triturated with diisopropyl ether to afford the title compounds as white solids (yield=64%).

¹HNMR (CDCl₃, 400 MHz) δ: 1.60-2.04 (m, 10H), 2.44 (m, 2H), 2.76 (m, 4H), 3.96 (s, 3H), 4.10-4.24 (m, 2H), 5.30 (m, 1H), 6.52 (s, 1H), 6.60 (d, 1H), 8.04 (m, 4H), 8.26 (m, 1H)

LRMS: m/z ES⁺ 526 [MNa]⁺

Microanalysis found; C, 59.45; H, 6.14; N, 8.05, C₂₅H₃₀FN₃O₅S; requires C, 59.63; H. 6.00; N, 8.34%.

### EXAMPLE 2

### Syn-5-Fluoro-N-(4-{[5-(2-methoxy-phenyl)-1H-pyrazole-3-carbonyl]-amino}-cyclohexyl)-2-(tetrahydro-thiopyran-4-yloxy)-nicotinamide

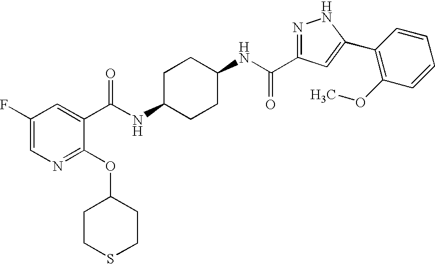

The amine hydrochloride from preparation 15a (325 mg, 0.83 mmol) was dissolved in dichloromethane, the solution washed with 1N sodium hydroxide solution, then dried (MgSO₄) and evaporated under reduced pressure.

1-(3-Dimethylaminopropyl)-3-ethylcarbodiimide hydrochloride (176 mg, 0.92 mmol) was added to a solution of the freshly prepared amine, 1-hydroxybenzotriazole hydrate (124 mg, 0.92 mmol), 5-(2-methoxy-phenyl)-2H-pyrazole-3-carboxylic acid (200 mg, 0.92 mmol) and N-methylmorpholine (101 μl, 0.92 mmol) in dichloromethane (5 ml), and the reaction stirred at room temperature for 18 hours. The mixture was partitioned between ethyl acetate (20 ml) and 2N hydrochloric acid (20 ml) and the layers separated. The aqueous phase was extracted with further ethyl acetate (20 ml), the combined organic extracts washed with sodium bicarbonate solution (20 ml), brine (20 ml), then dried (MgSO₄) and evaporated under reduced pressure. The crude product was purified by column chromatography on silica gel using ethyl acetate:pentane (50:50) as eluant to afford the title compound as white crystals, 273 mg.

¹HNMR (CDCl₃, 400 MHz) δ: 1.75-1.92 (m, 4H), 1.93-2.13 (m, 6H), 2.46 (m, 2H), 2.84 (m, 4H), 4.04 (s, 3H), 4.23 (brs, 2H), 5.30 (m, 1H), 7.08 (m, 2H), 7.21 (s, 1H), 7.35 (m, 2H), 7.71 (d, 1H), 8.03 (s, 1H), 8.10 (d, 1H), 8.26 (d, 1H)

LRMS: m/z ES⁺ 576 [MNa]⁺

### EXAMPLE 3

### Syn-7-Methoxy-imidazo[1,2-a]pyridine-8-carboxylic acid (4-{[5-fluoro-2-(tetrahydro-thiopyran-4-yloxy)-pyridine-3-carbonyl]-amino}-cyclohexyl)-amide

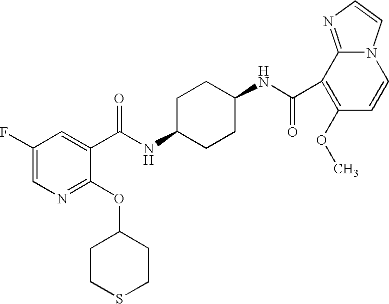

A mixture of the amine hydrochloride form preparation 15a (270 mg, 0.70 mmol), the acid from preparation 25 (125 mg, 0.65 mmol), O-(1H-benzotriazol-1-yl)-N,N,N′N′-tetramethyluronium hexafluorophosphate (152 mg, 0.65 mmol) and triethylamine (388 μl, 2.88 mmol) in N,N-dimethylformamide (5 ml) was stirred at room temperature for 24 hours. The solution was concentrated under reduced pressure and the residue diluted with 10% citric acid and extracted with ethyl acetate. The combined organic extracts were washed with sodium bicarbonate solution and brine, then dried (MgSO₄) and evaporated under reduced pressure. The crude product was purified by column chromatography on silica gel using an elution gradient of dichloromethane:methanol:0.88 ammonia (95:5:0.5 to 90:10:1) to afford the title compound, 70 mg.

¹HNMR (CD₃OD, 400 MHz) δ: 1.82-2.00 (m, 10H), 2.35-2.42 (m, 2H), 2.72 (m, 4H), 4.09 (m, 4H), 4.17 (m, 1H), 5.31 (m, 1H), 7.20 (d, 1H), 7.60 (s, 1H), 7.84 (s, 1H), 8.05 (m, 1H), 8.15 (d, 1H), 8.60 (d, 1H).

LRMS: m/z ES⁺ 528 [MH]⁺

### EXAMPLE 4

### Syn-5-Fluoro-N-{4-[2-(2-hydroxy-ethoxy)-benzoylamino]-cyclohexyl}-2-(tetrahydro-thiopyran-4-yloxy)-nicotinamide

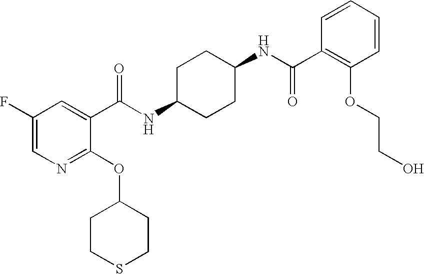

A mixture of the compound from preparation 80 (230 mg, 0.38 mmol), acetic acid (4 ml), water (1 ml) and tetrahydrofuran (2 ml) were stirred at 80° C. for 18 hours. The cooled reaction was partitioned between ether and saturated sodium bicarbonate solution, and the layers separated. The organic phase was washed with water, 2N hydrochloric acid, then dried (MgSO₄) and evaporated under reduced pressure. The residue was purified by column chromatography on silica gel using an elution gradient of ethyl acetate:pentane (50:50 to 100:0). The product was triturated with isopropyl ether to afford the title compound as a white solid, 161 mg.

¹HNMR (CD₃OD, 400 MHz) δ: 1.83-2.07 (m, 10H), 2.38 (m, 2H), 2.63-2.81 (m, 4H), 3.91 (t, 2H), 4.11 (m, 2H), 4.25 (t, 2H), 5.27 (m, 1H), 7.06 (m, 1H), 7.12 (m, 1H), 7.48 (m, 1H), 7.93 (m, 1H), 8.02 (m, 1H) 8.15 (m, 1H)

LRMS: m/z APCI⁺ 518 [MH]⁺

Microanalysis found; C, 60.30; H, 6.22; N, 8.09, C₂₂H₃₂FN₃O₅S requires C, 60.33; H, 6.23; N, 8.12%.

### EXAMPLE 5

### Syn-5-Fluoro-N-{5-[2-(2-hydroxy-ethoxy)-4-methyl-benzoylamino]-cyclohexyl}-2-(tetrahydro-thiopyran-4-yloxy)-nicotinamide

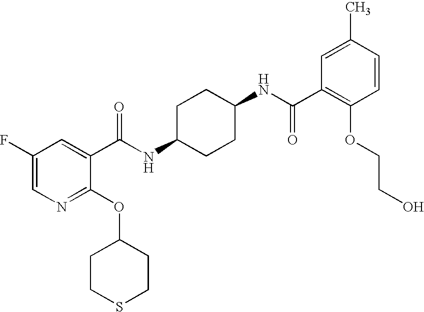

A mixture of the compound from preparation 86 (1.19 g, mmol) in water (5 ml), acetic acid (20 ml) and tetrahydrofuran (10 ml) was heated at 60° C. for 24 hours. The reaction mixture was evaporated under reduced pressure and the residue dissolved in ethyl acetate and washed with water, saturated sodium bicarbonate solution and brine, then dried (MgSO₄) and concentrated under reduced pressure. The residue was purified by column chromatography on silica gel using ethyl acetate and the product crystallised from isopropyl acetate to afford the title compound, 683 mg.

¹HNMR (CDCl₃, 400 MHz) δ: 1.60-2.04 (m, 10H), 2.34 (s, 3H), 2.42 (m, 2H), 2.78 (m, 4H), 4.04 (m, 2H), 4.10-4.30 (m, 4H), 5.26 (m, 1H), 6.86 (m, 1H), 7.22 (m, 1H), 7.98 (m, 1H), 8.04 (m, 1H), 8.10 (m, 1H), 8.16 (m, 1H), 8.28 (m, 1H)

LRMS: m/z APCI⁺ 532 [MH]⁺

### Further Method:

A mixture of the compound from preparation 86 (21.2 g, 34.5 mmol) in water (75 ml), acetic acid (300 ml) and tetrahydrofuran (150 ml) was heated at 70° C. for 24 hours. The reaction mixture was evaporated under reduced pressure and the residue dissolved in ethyl acetate and washed with water, saturated sodium bicarbonate solution and brine, then dried (MgSO₄) and concentrated under reduced pressure. The residue was dissolved in methanol (200 ml), tosic acid (1 g) added and the mixture stirred at room temperature for 1.5 hours. The solution was poured into ethyl acetate and washed with saturated sodium bicarbonate solution, and brine, then dried (MgSO₄) and evaporated under reduced pressure. The resulting solid was recrystallised from isopropyl acetate to afford the title compound as a white solid, 14.12 g.

¹HNMR (CD₃OD, 400 MHz) δ: 1.89 (m, 8H), 2.01 (m, 2H), 2.32 (s, 3H), 2.38 (m, 2H), 2.74 (m, 4H), 3.94 (t, 2H), 4.10 (m, 2H), 4.24 (t, 2H), 5.28 (m, 1H), 7.03 (d, 1H), 7.29 (dd, 1H), 7.77 (d, 1H), 8.02 (dd, 1H), 8.14 (d, 1H)

LRMS: m/z APCI⁺ 532 [MH]⁺

Microanalysis found: C, 60.18; H. 6.42; N, 7.75. C₂₇H₃₄FN₃O₅S;0.4H₂O requires C, 60.18; H, 6.51; N. 7.80%.

### EXAMPLES 6 to 12

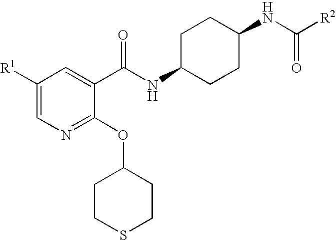

1-(3-Dimethylaminopropyl)-3-ethylcarbodilmide hydrochloride (1.5-2 eq) was added to a solution of the appropriate amine (preparation 15a and 18) (1-1.5 eq), 1-hydroxybenzotriazole hydrate (1.25 eq), the appropriate acids from preparation 58 to 63 (1 eq) and N-ethyldiisopropylamine (3 eq) in N,N-dimethylformamide (3-4 mlmmol⁻¹), and the reaction stirred at room temperature for 18 hours. The mixture was partitioned between ethyl acetate and 10% citric acid solution, and the layers separated. The organic phase was washed with water, sodium carbonate solution, dried (MgSO₄) and evaporated under reduced pressure. The residue was dissolved in tetrahydrofuran (2.5 mlmmol⁻¹), and acetic acid (5 mlmmol⁻¹) and water (1.25 mlmmol⁻¹) added, and the solution stirred at 70° C. for 18 hours. The mixture was partitioned between ethyl acetate and water, the layers separated, and the organic phase washed with sodium bicarbonate solution, water and brine, then dried (MgSO₄) and evaporated under reduced pressure. The crude products were purified by column chromatography on silica gel using either acetonitrile:dichloromethane (20:80 to 100:0) or ethyl acetate:pentane (30:70 to 100:0) elution gradients, then triturated from isopropyl ether to afford the title compounds as white solids.

| Ex | | | Yield | |
|---|---|---|---|---|
| No | R¹ | R² | (%) | Data |
| 6 | F | 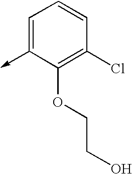 | 51 | ¹HNMR (CDCl₃, 400 MHz) δ: 1.62-2.04 (m,10H), 2.44 (m, 2H), 2.80 (m, 4H), 2.80-3.20 (brs,1H), 4.00 (m, 2H), 4.10-4.25 (m, 4H), 5.30 (m,1H), 7.16 (m, 1H), 7.50 (m, 1H), 7.80 (m, 1H),7.92 (m, 1H), 8.02 (m, 1H), 8.12 (m, 1H), 8.22(m, 1H)LRMS: m/z ES⁺ 574 [MNa]⁺Microanalysis found; C, 56.54; H, 5.67; N, 7.54,C₂₆H₃₁ClFN₃O₅S; requires C, 56.57; H, 5.66; N,7.61%. |
| 7 | F | 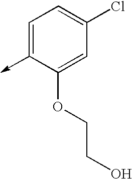 | 56 | ¹HNMR (CDCl₃, 400 MHz) δ: 1.68-1.96 (m,10H), 2.40 (m, 2H), 2.78 (m, 4H), 2.80-3.15 (brs,1H), 4.04 (m, 2H), 4.12 (m, 2H), 4.22 (m, 2H),5.24 (m, 1H), 6.92 (m, 1H), 7.00 (m, 1H),8.00-8.22 (m, 5H)LRMS: m/z ES⁺ 574 [MNa]⁺Microanalysis found; C, 56.24; H, 5.63; N, 7.60,C₂₆H₃₁ClFN₃O₅S; requires C, 56.57; H, 5.66; N,7.61%. |
| 8 | F | 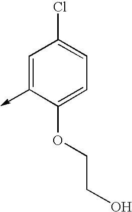 | 64 | ¹HNMR (CDCl₃, 400 MHz) δ: 1.68-1.96 (m,10H), 2.40 (m, 2H), 2.78 (m, 4H), 2.80-3.15 (brs,1H), 4.04 (m, 2H), 4.12 (m, 2H), 4.22 (m, 2H),5.24 (m, 1H), 6.92 (m, 1H), 7.00 (m, 1H),8.00-8.22 (m, 5H)LRMS: m/z ES⁺ 574 [MNa]⁺Microanalysis found; C, 56.24; H, 5.63; N, 7.60,C₂₆H₃₁ClFN₃O₅S; requires C, 56.57; H, 5.66; N,7.61%. |
| 9 | F | 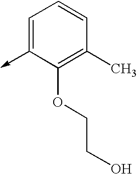 | 43 | ¹HNMR (CDCl₃, 400 MHz) δ: 1.60-2.06 (m,10H), 2.34, (s, 3H), 2.44 (m, 2H), 2.78 (m, 4H),3.92-4.02 (m, 4H), 4.14-4.30 (m, 2H), 5.30 (m,1H), 7.14 (t, 1H), 7.32 (d, 1H), 7.70 (d, 1H), 7.84(m, 1H), 8.04 (d, 1H), 8.14 (d, 1H), 8.25 (m, 1H)LRMS: m/z ES⁺ 554 [MNa]⁺Microanalysis found; C, 60.09; H, 6.52; N, 7.77,C₂₇H₃₄FN₃O₅S; 0.45H₂O requires C, 60.08; H,6.52; N, 7.78%. |
| 10 | F | 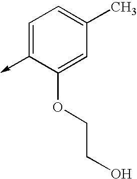 | 66 | ¹HNMR (CDCl₃, 400 MHz) δ: 1.68-2.10 (m,10H), 2.38 (s, 3H), 2.44 (m, 2H), 2.78 (m, 4H),4.04 (m, 2H), 4.10-4.30 (m, 4H), 5.28 (m, 1H),6.76 (m, 1H), 6.92 (m, 1H), 8.00-8.18 (m, 4H),8.26 (m, 1H)LRMS: m/z ES⁺ 554 [MNa]⁺Microanalysis found; C, 60.53; H, 6.47; N, 7.84,C₂₇H₃₄FN₃O₅S; 0.2H₂O requires C, 60.59; H,6.48; N, 7.85%. |
| 11^{A} | H | 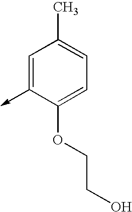 | 40 | ¹HNMR (CD₃OD, 400 MHz) δ: 1.80-2.06 (m,10H), 2.31 (s, 3H), 2.38 (m, 2H), 2.65-2.81 (m,4H), 3.92 (t, 2H), 4.11 (m, 2H), 4.22 (t, 2H), 5.37(m, 1H), 7.05 (m, 2H), 7.29 (d, 1H), 7.77 (s, 1H),8.25 (m, 2H).LRMS: m/z APCI⁺ 514 [MH]⁺Microanalysis found; C, 62.96; H, 6.86; N, 8.05.C₂₇H₃₅N₃O₅S requires C, 63.14; H, 6.87; N,8.18%. |
| 12^{A} | H | 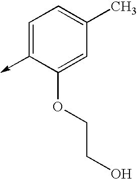 | 54 | ¹HNMR (CD₃OD, 400 MHz) δ: 1.80-2.05 (m,10H), 2.38 (m, 5H), 2.65-2.81 (m, 4H), 3.93 (t,2H), 4.10 (m, 2H), 4.24 (t, 2H), 5.37 (m, 1H),6.88 (d, 1H), 6.97 (s, 1H) 7.09 (m, 1H), 7.84 (d,1H), 8.22 (m, 2H)LRMS: m/z APCI⁺ 514 [MH]⁺Microanalysis found; C, 62.61; H, 6.90; N, 7.96.C₂₇H₃₅N₃O₅S; 0.25 H₂O requires C, 62.59; H,6.91; N, 8.11%. |

A = triethylamine was used instead of N-ethyldiisopropylamine

### EXAMPLE 13

### Syn-N-{4-[2-(2-Hydroxy-ethoxy)-benzoylamino]-cyclohexyl}-5-methyl-2-(tetrahydro-thiopyran-4-yloxy)-nicotinamide

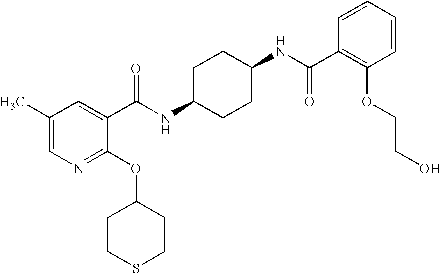

1-(3-Dimethylaminopropyl)-3-ethylcarbodiimide hydrochloride (112 mg, 0.59 mmol) was added to a mixture of the amine from preparation 17 (150 mg, 0.39 mmol), the acid from preparation 64 (105 mg, 0.39 mmol), 1-hydroxybenzotriazole hydrate (52 mg, 0.39 mmol) and N-ethyldiisopropylamine (135 μl, 0.78 mmol) in N,N-dimethylformamide (2 ml), and the reaction stirred at room temperature for 18 hours. The mixture was partitioned between dichloromethane (10 ml) and 2N hydrochloric acid (20 ml) and the layers separated. The organic layer was concentrated under reduced pressure and the residue taken up in a solution of tetrahydrofuran (2 ml), water (1 ml) and acetic acid (4 ml), and the solution stirred at 85° C. for 24 hours. The reaction was basified by the addition of solid potassium carbonate, the mixture diluted with water and ethyl acetate, and the layers separated. The organic phase was dried (MgSO₄) and evaporated under reduced pressure.

The crude product was purified by HPLC using a Phenomenex C₁₈ column, and an elution gradient of 0.1% aqueous trifluoroacetic acid:acetonitrile (95:5 to 0:100) to give the title compound.

HRMS: m/z APCI⁺ 514.2353 [MH]⁺ req 514.2370

### EXAMPLE 14

### Syn-5-Fluoro-N-{4-[2-(3-hydroxy-propoxy)-benzoylamino]-cyclohexyl}-2-(tetrahydro-thiopyran-4-yloxy)-nicotinamide

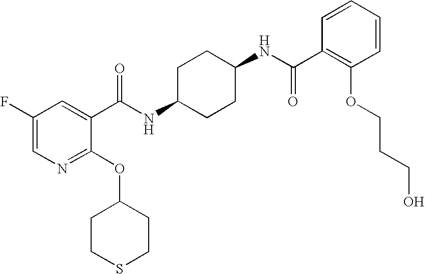

Potassium carbonate (130 mg, 0.94 mmol), potassium iodide (8.3 mg, 0.05 mmol) and 1-tetrahydropyranyloxy-3-bromopropane (105 μl, 0.62 mmol) were added to a solution of the phenol from preparation 89 (226 mg, 0.47 mmol) in acetonitrile (6 ml) and N,N-dimethylformamide (1 ml) and the reaction stirred at 90° C. for 4 hours. The mixture was partitioned between ethyl acetate and 10% citric acid, and the layers separated. The organic layer was washed with water, sodium bicarbonate solution and brine, then dried (MgSO₄) and evaporated under reduced pressure. The residue was dissolved in acetic acid:tetrahydrofuran:water (4 ml:2 ml:1 ml), and the solution stirred at 70° C. for 18 hours. The reaction was partitioned between ethyl acetate and water, the layers separated, and the organic phase washed with sodium bicarbonate solution, water and brine, then dried (MgSO₄) and evaporated under reduced pressure. The crude product was purified by column chromatography on silica gel using an elution gradient of ethyl acetate:pentane (40:60 to 100:0) and the product triturated with diisopropyl ether to afford the title compound as a white solid, 85 mg.

¹HNMR (CDCl₃, 400 MHz) δ: 1.60-2.00 (m, 10H), 2.14 (m, 2H), 2.42 (m, 2H), 2.64-2.80 (m, 4H), 3.80 (t, 2H), 4.08 (m, 2H), 4.30 (t, 2H), 5.24 (m, 1H), 6.94-7.06 (m, 2H), 7.38 (t, 1H), 8.04 (m, 3H), 8.10 (m, 1H), 8.20 (m, 1H)

LRMS: m/z ES⁺ 554 [MNa]⁺

Microanalysis found; C, 60.85; H, 6.51; N, 7.76, C₂₇H₃₄FN₃O₅S; requires C, 61.00; H, 6.45; N, 7.90%.

### EXAMPLES 15 to 21

The following compounds of general formula:

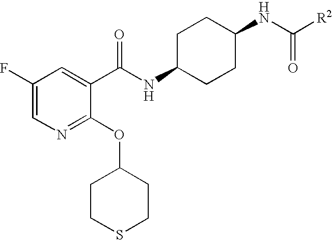

were prepared as white solids from the appropriate phenols (examples 37-40 and 48) and 2-(2-bromoethoxy)tetrahydro-2-pyran or 1-tetrahydropyranyloxy-3-bromopropane following the procedure described in example 14.

| Ex | | Yield | |
|---|---|---|---|
| No | R² | (%) | Data |
| 15¹ | 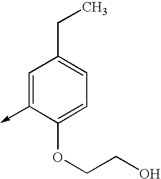 | 47 | ¹HNMR (CDCl₃, 400 MHz) δ: 1.22 (m, 3H),1.64-2.04 (m, 10H), 2.10 (m, 2H), 2.60 (q, 2H), 2.76(m, 4H), 3.20-3.50 (brs, 1H), 4.00 (m, 2H), 4.10 (m,2H), 4.22 (m, 2H), 5.22 (m, 1H), 6.94 (d, 1H), 7.22(m, 1H), 7.96 (m, 1H), 8.00 (m, 1H), 8.06 (d, 1H),8.16-2.26 (m, 2H)LRMS: m/z ES⁺ 546 [MH]⁺Microanalysis found; C, 61.45; H, 6.73; N, 7.46,C₂₈H₃₆FN₃O₅S; requires C, 61.63; H, 6.65; N, 7.70%. |
| 16² | 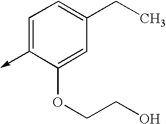 | 50 | ¹HNMR (CDCl₃, 400 MHz) δ: 1.22 (m, 3H),1.64-2.04 (m, 10H), 2.42 (m, 2H), 2.66 (q, 2H), 2.78(m, 4H), 3.00-3.30 (brs, 1H), 4.04 (t, 2H), 4.14 (m,2H), 4.26 (t, 2H), 5.24 (m, 1H), 6.76 (s, 1H), 6.90 (d,1H), 8.00-8.12 (m, 3H), 8.14-8.26 (m, 2H)LRMS: m/z ES⁺ 568 [MNa]⁺Microanalysis found; C, 61.25; H, 6.69; N, 7.67,C₂₈H₃₆FN₃O₅S; 0.2H₂O requires C, 61.23; H, 6.68; N,7.65%. |
| 17³ | 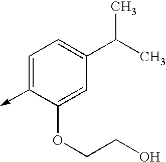 | 41 | ¹HNMR (CDCl₃, 400 MHz) δ: 1.24 (d, 6H), 1.64-2.00(m, 10H), 2.42 (m, 2H), 2.76 (m, 4H), 2.88 (m, 1H),3.20-3.60 (brs, 1H), 4.04 (m, 2H), 4.10 (m, 2H), 4.26(m, 2H), 5.22 (m, 1H), 6.78 (s, 1H), 6.92 (d, 1H),8.00-8.10 (m, 3H), 8.20 (m, 2H)LRMS: m/z ES⁺ 582 [MNa]⁺Microanalysis found; C, 61.86; H, 6.88; N, 7.38,C₂₉H₃₈FN₃O₅S; 0.2H₂O requires C, 61.84; H, 6.87; N,7.46%. |
| 18⁴ | 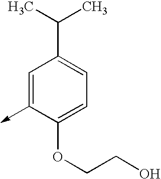 | 56 | ¹HNMR (CDCl₃, 400 MHz) δ: 1.24 (d, 6H), 1.64-2.00(m, 10H), 2.42 (m, 2H), 2.76 (m, 4H), 2.90 (m, 1H),3.20-3.40 (brs, 1H), 4.02 (m, 2H), 4.08 (m, 2H), 4.26(m, 2H), 5.24 (m, 1H), 6.78 (s, 1H), 6.94 (d, 1H),8.00-8.10 (m, 3H), 8.20 (m, 2H)LRMS: m/z ES⁺ 582 [MNa]⁺Microanalysis found; C, 61.84; H, 6.85; N, 7.43,C₂₉H₃₈FN₃O₅S; 0.2H₂O requires C, 61.84; H, 6.87; N,7.46%. |
| 19 | 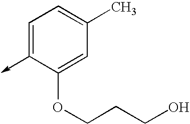 | 30 | ¹HNMR (CDCl₃, 400 MHz) δ: 1.64-2.04 (m, 10H),2.12 (m, 2H), 2.36-2.50 (m, 5H), 2.78 (m, 4H), 3.86(t, 2H), 4.08-4.22 (m, 2H), 4.30 (t, 2H), 5.26 (m, 1H),6.80 (s, 1H), 6.88 (d, 1H), 7.94 (m, 1H), 8.06 (m, 3H),8.26 (m, 1H)LRMS: m/z ES⁺ 568 [MNa]⁺Microanalysis found; C, 61.16;.H, 6.57; N, 7.58,C₂₈H₃₆FN₃O₅S; 0.2H₂O requires C, 61.23; H, 6.68; N,7.65%. |
| 20 | 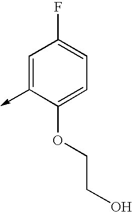 | 54 | ¹HNMR (DMSO-d₆ 400 MHz) δ: 1.70 (m, 8H), 1.86(m, 2H), 2.25 (m, 2H), 2.62 (m, 2H), 2.72 (m, 2H),3.74 (q, 2H), 3.92 (m, 2H), 4.15 (t, 2H), 4.98 (t, 1H),5.14 (m, 1H), 7.20 (m, 1H), 7.32 (m, 1H), 7.58 (dd,1H), 7.96 (dd, 1H), 8.05 (d, 1H), 8.27 (d, 1H), 8.40(d, 1H).LRMS: m/z ES³⁰ 558 [MNa⁺]Microanalysis found: C, 58.20; H, 5.87; N, 7.75.C₂₆H₃₁F₂N₃O₅S requires C, 58.30; H, 5.83; N, 7.85%. |
| 21 | 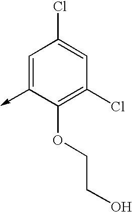 | 49 | ¹HNMR (DMSO-d₆ 400 MHz) δ: 1.70 (m, 8H), 1.85(m, 2H), 2.27 (m, 2H), 2.64 (m, 2H), 2.75 (m, 2H),3.65 (q, 2H), 3.85 (m, 1H), 3.95 (m, 1H), 4.02 (t, 2H),4.95 (t, 1H), 5.16 (m, 1H), 7.46 (s, 1H), 7.74 (s, 1H),7.96 (m, 1H), 8.07 (d, 1H), 8.26 (d, 1H), 8.40 (d, 1H).LRMS: m/z ES⁺ 608 [MNa⁺]Microanalysis found: C, 53.26; H, 5.23; N, 7.03.C₂₆H₃₀Cl₂FN₃O₅S requires C, 53.25; H, 5.16; N,7.16%. |

### EXAMPLE 22

### Syn-N-[4-(2-Ethoxy-4-hydroxy-benzoylamino)cyclohexyl]-5-fluoro-2-(tetrahydro-thiopyran-4-yloxy)-nicotinamide

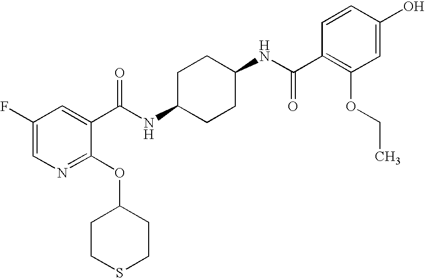

Palladium black (180 mg) was added to a solution of the compound from preparation 83 (172 mg, 0.28 mmol) in formic acid (20 ml, 4.4% in methanol) and N,N-dimethylformamide (2 ml), and the reaction stirred under nitrogen for 18 hours. TLC analysis showed starting material remaining, so formic acid (0.72 ml) and additional palladium black (90 mg) were added and the mixture stirred for a further 48 hours. The reaction was filtered, the filtrate evaporated under reduced pressure and the residue partitioned between sodium bicarbonate and ethyl acetate. The organic phase was washed with water and brine, then dried (MgSO₄) and concentrated under reduced pressure. The crude product was purified by column chromatography on silica gel using an elution gradient of ethyl acetate:pentane (40:60 to 100:0) to afford the title compound as a white solid, 45 mg.

¹HNMR (CDCl₃, 400 MHz) δ: 1.50 (t, 3H), 1.60-2.06 (m, 10H), 2.42 (m, 2H), 2.74 (m, 4H), 4.04-4.24 (m, 4H), 5.24 (m, 1H), 6.54 (s, 1H), 6.62 (m, 1H), 8.06 (m, 3H), 8.24 (m, 2H), 9.08-9.36 (m, 1H)

LRMS: m/z ES⁺ 540 [MNa]⁺

Microanalysis found; C, 58.56; H, 6.29; N, 7.81, C₂₆H₃₂FN₃O₅S; 0.85H₂O requires C, 58.60; H. 6.37; N, 7.88%.

### EXAMPLE 23

### Syn-N-[4-(2-Cyclopropylmethoxy-4-hydroxy-benzoylamino)-cyclohexyl]-5-fluoro-2-(tetrahydro-thiopyran-4-yloxy)-nicotinamide

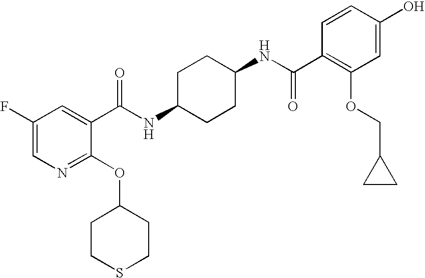

The title compound was obtained as a white solid in 59% yield from the compound from preparation 84, following the procedure described in example 22.

¹HNMR (CDCl₃, 400 MHz) δ: 0.40 (m, 2H), 0.64 (m, 2H), 1.24-1.38 (m, 1H), 1.62-2.04 (m, 10H), 2.42 (m, 2H), 2.76 (m, 4H), 3.92 (d, 2H), 4.04-4.22 (m, 2H), 5.24 (m, 1H), 6.44 (s, 1H), 6.58 (d, 1H), 8.06 (m, 3H), 8.26 (m, 1H), 8.40 (d, 1H)

LRMS: m/z ES⁺ 566 [MNa]⁺

Microanalysis found; C, 60.87; H, 6.37; N, 7.64, C₂₈H₃₄FN₃O₅S; 0.5H₂O requires C, 60.85; H, 6.38; N, 7.60%.

### EXAMPLE 24

### Syn-N-[4-(2-Cyclopentoxy-4-hydroxy-benzoylamino)cyclohexyl]-5-fluoro-2-(tetrahydro-thiopyran-4-yloxy)-nicotinamide

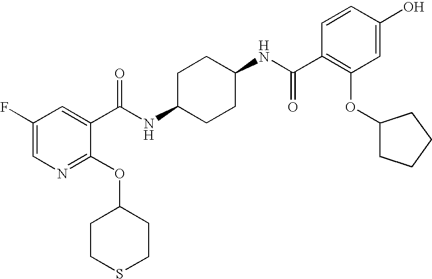

The title compound was obtained as a white solid in 60% yield from the compound from preparation 85, following the procedure described in example 22.

¹HNMR (CD₃OD, 400 MHz) δ: 1.64-2.12 (m, 18H), 2.20 (m, 2H), 2.68-2.82 (m, 4H), 4.00-4.14 (m, 2H), 5.02 (m, 1H), 5.30 (m, 1H), 6.46 (m, 1H), 6.52 (m, 1H), 7.84 (m, 1H), 8.04 (m, 1H), 8.16 (m, 1H), 8.22 (m, 1H)

LRMS: m/z ES³⁰ 580 [MNa]⁺

Microanalysis found; C, 62.12; H, 6.50; N. 7.43, C₂₉H₃₆FN₃O₅S requires C, 62.46; H, 6.51; N, 7.53%.

### EXAMPLES 25 and 26

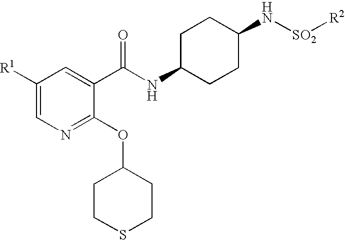

A mixture of the appropriate amine hydrochloride from preparations 15a and 18 (1 eq), the appropriate sulphonyl chlorides (1.3 eq) and triethylamine (3 eq) in dichloromethane (25 mlmmol⁻¹) was stirred at room temperature for 18 hours. The solution was washed with 10% citric acid solution then evaporated under reduced pressure. The product was crystallised from isopropyl acetate, to afford the title compounds as solids.

| Ex | | | Yield | |
|---|---|---|---|---|
| no | R¹ | R² | (%) | Data |
| 25 | F | 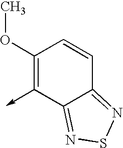 | 95 | ¹HNMR (DMSO-d₆, 400 MHz) δ: 1.51 (m, 6H),1.66 (m, 2H), 1.90 (m, 2H), 2.27 (m, 2H), 2.69(m, 2H), 2.76 (m, 2H), 3.22 (m, 1H), 3.77 (m,1H), 4.14 (s, 3H), 5.16 (m, 1H), 7.49 (d, 1H),7.90 (d, 1H), 7.98 (dd, 1H), 8.03 (d, 1H), 8.28 (d,1H), 8.35 (d, 1H).LRMS: m/z (APCI⁺) 604 [MNa]⁺Microanalysis found: C, 48.89; H, 5.31; N, 11.49.C₂₄H₂₈FN₅O₅S₃; 0.4H₂O requires C, 48.95; H,4.93; N, 11.89%. |
| 26^{B} | H | 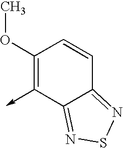 | 57 | ¹HNMR (DMSO-d₆, 400 MHz) δ: 1.52 (m, 6H),1.67 (m, 2H), 1.90 (m, 2H), 2.32 (m, 2H),2.70-2.82 (m, 4H), 3.23 (m, 1H), 3.76 (m, 1H),4.13 (s, 3H), 5.25 (m, 1H), 7.10 (dd, 1H), 7.49(d, 1H), 7.90 (d, 1H), 7.93 (d, 1H), 8.11 (d, 1H),8.25 (m, 1H), 8.35 (d, 1H).LRMS: m/z (APCI⁺) 586 [MNa]⁺Microanalysis found: C, 50.60; H, 5.11; N, 12.23.C₂₄H₂₉N₅O₅S₃; 0.1H₂O requires C, 50.97; H,5.20; N, 12.38%. |

B = compound additionally purified by column chromatography on silica gel using dichloromethane:methanol (99:1).

### EXAMPLE 27

### Syn-N-[4-(2-methoxy-5-methyl-benzenesulfonylamino)-cyclohexyl]-2-(tetrahydro-thiopyran-4-yloxy)-nicotinamide

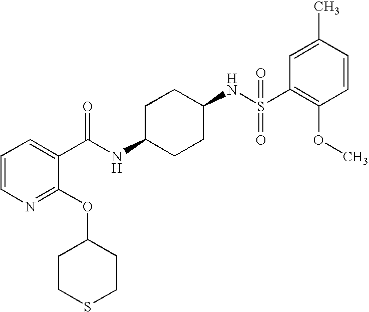

The amine hydrochloride from preparation 18 (500 mg, 1.34 mmol) was dissolved in dichloromethane, the solution washed with 1N sodium hydroxide solution, then dried (MgSO₄) and evaporated under reduced pressure.

1-(3-Dimethylaminopropyl)-3-ethylcarbodiimide hydrochloride (385 mg, 2.0 mmol) was added to a solution of this amine, 1-hydroxybenzotriazole hydrate (181 mg, 1.34 mmol), 6-methoxy-m-toluenesulphonylchloride (267 mg, 1.21 mmol) and N-ethyldiisopropylamine (934 μl, 5.36 mmol) in N,N-dimethylformamide (5 ml), and the reaction stirred at room temperature for 18 hours. The mixture was evaporated under reduced pressure and the residue purified by column chromatography on silica gel using ethyl acetate:pentane (50:50) to afford the title compound as a white solid, 317 mg.

¹HNMR (CDCl₃, 400 MHz) δ: 1.53-1.72 (m, 6H), 1.74-1.87 (m, 2H), 1.90-2.02 (m, 2H), 2.33 (s, 3H), 2.38-2.49 (m, 2H), 2.72-2.86 (m, 4H), 3.23 (brs, 1H), 3.89-4.04 (m, 4H), 5.10 (d, 1H), 5.36 (m, 1H), 6.91 (d, 1H), 7.01 (m, 1H), 7.31 (d, 1H), 7.64 (s, 1H), 7.93 (d, 1H), 8.18 (d, 1H), 8.47 (d, 1H)

LRMS: m/z ES⁺ 542 [MNa]⁺

### EXAMPLE 28

### Syn-5-Fluoro-N-[4-(7-methoxy-quinoline-8-sulfonylamino)-cyclohexyl]-2-(tetrahydro-thiopyran-4-yloxy)-nicotinamide

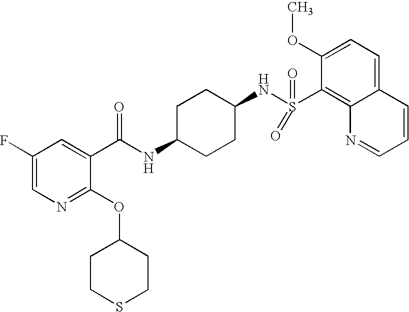

Chlorosulphonic acid (0.21 ml, 3.2 mmol) was added dropwise to ice-cooled 7-methoxyquinoline (Syn. Comm. 2000; 30(2); 367) (100 mg, 0.63 mmol), and the solution then heated to 100° C. for 1 hour. The cooled mixture was poured onto ice, sodium bicarbonate slowly added, followed by acetonitrile (30 ml) and the amine from preparation 15a (171 mg, 0.44 mmol). Triethylamine (0.2 ml, 1.44 mmol) was then added and the solution stirred at room temperature for 18 hours. The solution was evaporated under reduced pressure and the residue partitioned between dichloromethane and water. The organic layer was evaporated under reduced pressure and the residue purified by column chromatography on silica gel using dichloromethane:methanol (98:2) to give the title compound as a white solid, 154 mg.

¹HNMR (DMSO-d₆, 400 MHz) δ: 1.42 (m, 4H), 1.54 (m, 4H), 1.86 (m, 2H), 2.23 (m, 2H), 2.66 (m, 2H), 2.77 (m, 2H), 3.25 (m, 1H), 3.75 (m, 1H), 4.03 (s, 3H), 5.16 (m, 1H), 7.53 (dd, 1H), 7.60 (d, 1H), 7.70 (d, 1H), 7.89 (dd, 1H), 8.01 (d, 1H), 8.26 (m, 2H), 8.45 (d, 1H), 8.98 (dd, 1H).

LRMS: m/z (APCI⁻) 573 [M-H]⁻

### EXAMPLE 29

### Syn-N-[4-(7-methoxy-quinoline-8-sulfonylamino)-cyclohexyl]-2-(tetrahydro-thiopyran-4-yloxy)-nicotinamide

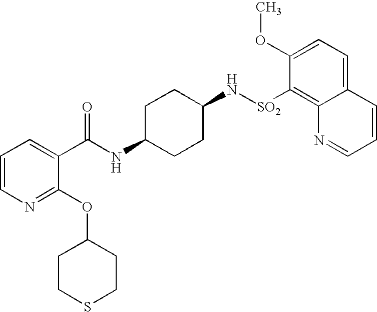

The title compound was obtained as white crystals from the amine from preparation 18 and 7-methoxyquinoline (Syn. Comm. 2000; 30(2); 367) following the procedure described in example 28.

¹HNMR (DMSO-d₆, 400 MHz) δ: 1.40 (m, 4H), 1.52 (m, 4H), 1.88 (m, 2H), 2.24 (m, 2H), 2.67 (m, 2H), 2.76 (m, 2H), 3.25 (m, 1H), 3.74 (m, 1H), 4.02 (s, 3H), 5.24 (m, 1H), 7.08 (dd, 1H), 7.52 (dd, 1H), 7.60 (d, 1H), 7.70 (d, 1H), 7.92 (dd, 1H), 8.03 (d, 1H), 8.25 (m, 2H), 8.45 (d, 1H), 8.99 (dd, 1H).

LRMS: m/z (APCI⁺) 579 [MNa]⁺

Microanalysis found: C, 56.88; H, 5.87; N, 9.80. C₂₇H₃₂N₄O₅S₂;0.6H₂O requires C, 57.14; H, 5.90; N, 9.87%.

### PREPARATION 1

### 2-Chloro-5-fluoro nicotinic acid

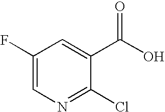

Ethyl-2-chloro-5-fluoro-nicotinoate (50:4 g, 0.247 mol) (see reference J. Med. Chem., 1993, 36(18), 2676-88) was dissolved in tetrahydrofuran (350 ml) and a 2M aqueous solution of lithium hydroxide (247 ml, 0.495 mol) added. The reaction mixture was stirred at room temperature for 3 days. The pH of the solution was reduced to pH1 by addition of 6 N hydrochloric acid and then extracted with dichloromethane (3×). The combined extracts were dried (MgSO₄) and the solvent evaporated under reduced pressure to give a solid which was triturated with diethyl ether and then dried to give the title compound (40.56 g) as a white solid.

¹H NMR (400 MHz, DMSO-d₆): δ 8.20 (s, 1H), 8.62 (s, 1H)

LRMS (ES⁺): m/z [MH]⁺174.

### PREPARATION 2

### Trans-N-tert-butyl (4-hydroxy-cyclohexyl)-carbamate

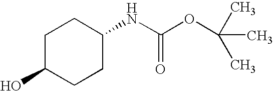

Trans-4-aminocyclohexanol (100 g, 0.87 mol) was added to acetonitrile (1 L), with stirring followed by di-tert-butyl dicarbonate (208 g, 0.96 mol) in portions over 1 hour. The reaction was stirred at room temperature for 18 hours, the resulting precipitate filtered off, and washed with ethyl acetate:hexane (1:3, 250 ml), then hexane (250 ml) and dried to afford the title compound as a white solid, 166.9 g.

m.p.-167-170° C.

### PREPARATION 3

### Trans-Methanesulphonic acid 4-tert-butoxycarbonylamino-cyclohexyl ester

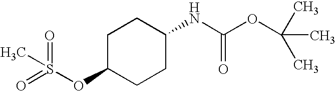

A solution of mesyl chloride (122.4 g, 1.07 mol) in dichloromethane (400 ml) was added dropwise over 45 minutes to an ice-cooled solution of the alcohol from preparation 2 (200 g, 0.93 mol) and triethylamine (112.8 g, 1.115 mol) in dichloromethane (1 L). The reaction was stirred for 15 minutes, then allowed to warm to room temperature over 1 hour. The mixture was washed with water (3×1.5 L), then stirred with silica (100 ml, Merck 60H). This mixture was filtered and the filtrate concentrated under reduced pressure to approx quarter volume. Hexane (500 ml) was added, the mixture cooled to 0° C., the resulting solid filtered off, dried and recrystallised from ethyl acetate to give the title compound, 221.1 g.

m.p.-146-148° C.

### PREPARATION 4

### syn-(4-Azido-cyclohexyl)-carbamic acid tert-butyl ester

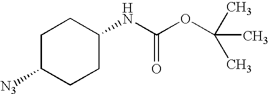

Sodium azide (25.5 g, 0.39 mol) was added to a solution of the mesylate from preparation 3 (100 g, 0.34 mol) in N,N-dimethylformamide (500 ml) and the reaction slowly warmed to 80° C. and stirred for a further 24 hours at this temperature. Ice/water (1 L) was added slowly to the cooled reaction, and the resulting precipitate was filtered off, washed with water and dried. The solid was dissolved in ethyl acetate (200 ml), the solution washed with water, dried (MgSO₄) and evaporated under reduced pressure. The residual solid was recrystallised from hexane to afford the title compound as a white solid, 50.8 g.

m.p.-79-81° C.

### PREPARATION 5

### Syn-tert-Butyl 4-aminocyclohexylcarbamate

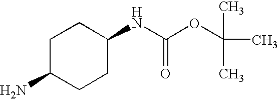

5% Palladium on charcoal (5 g) was mixed with toluene (10 ml) and was added to the azide from preparation 4 (170 g, 0.71 mol) in methanol (400 ml). The mixture was hydrogenated (80 atmospheres) at room temperature for 18 hours and then filtered. The solvent was evaporated in-vacuo and the residue was triturated with ethyl acetate (50 ml) and then with hexane (200 ml). The solid obtained was isolated by filtration, dissolved in ethyl acetate (600 ml) and filtered through Celite®. The filtrate was concentrated in-vacuo to give a slush that was diluted with hexane (300 ml). The solid obtained was isolated by filtration and was washed with ethyl acetate in hexane (20:80). The mother liquors were combined and evaporated in-vacuo, the residue was purified by chromatography on silica gel using ethyl acetate and then methanol as eluant. The material obtained was crystallised from ethyl acetate and hexane and combined with the first crop to give the title compound as a white solid (76 g).

Mp 88-90° C.

### PREPARATION 6

### Syn-{4-[(2-Chloro-5-fluoropyridine-3-carbonyl)amino]-cyclohexyl}-carbamic acid tert-butyl ester

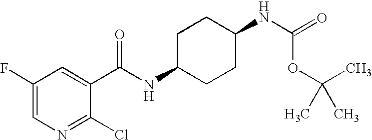

Oxalyl chloride (8 ml, 90 mmol) was added over 10 minutes to an ice-cooled suspension of the acid from preparation 1 (10 g, 57 mmol) and N,N-dimethylformamide (5 drops) in dichloromethane (200 ml). The suspension was then stirred at room temperature for 3 hours, and concentrated under reduced pressure. The residue was azeotroped with dichloromethane to give the intermediate acid chloride as a white solid.

This was dissolved in dichloromethane (200 ml), the solution cooled in a water bath, then N-diisopropyl-ethylamine (20 ml, 115 mmol) and the amine from preparation 5 (13.4 g, 62 mmol) added. The reaction mixture was stirred for 18 hours, diluted with dichloromethane (100 ml) and washed sequentially with 10% citric acid solution, saturated sodium bicarbonate solution (×2), water then brine. The organic solution was dried (MgSO₄) and evaporated under reduced pressure to afford the title compound as a yellow foam, 20.2 g.

¹H NMR (400 MHz, CDCl₃): δ 1.27 (s, 9H), 1.76 (m, 2H), 1.86 (m, 6H), 3.64 (m, 1H), 4.16 (m, 1H), 4.54 (m, 1H), 6.67 (s, 1H), 7.80 (m, 1H), 8.33 (d, 1H).

LRMS: m/z ES⁺ 394 [MNa]⁺

### PREPARATION 7

### Syn-{4-[(2,5-Dichloro-pyridine-3-carbonyl)-amino]-cyclohexyl}carbamic acid tert-butyl ester

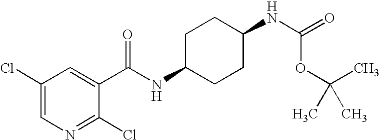

Carbonyl diimidazole (1.7 g, 10.5 mmol), was added to a solution of 2,5-dichloronicotinic acid (WO 95/30676, pg 19, method 1b) (2 g, 10.55 mmol) in N,N-dimethylformamide (20 ml), and the solution stirred at room temperature for 1 hour. The amine from preparation 5 (2.46 g, 11.5 mmol) was added and the reaction stirred at room temperature for 3 days. The mixture was concentrated under reduced pressure and the residue partitioned between 10% citric acid solution and ether. The layers were separated, the organic washed with further 10% citric acid solution, water, saturated sodium bicarbonate solution and brine. The solution was dried (MgSO₄) and evaporated under reduced pressure to afford the title compound as a white foam, 3.61 g.

¹HNMR (CDCl₃, 400 MHz) δ: 1.43 (s, 9H), 1.44-1.92 (m, 8H), 3.63 (m, 1H), 4.17 (m, 1H), 4.54 (m, 1H), 6.55 (m, 1H), 8.14 (s, 1H), 8.42 (s, 1H)

LRMS: m/z ACPI⁻ 388 [M-H]⁻

### PREPARATION 8

### 2-Chloro-5-methylnicotinic acid

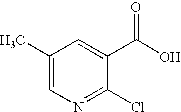

2,2,6,6-Tetramethylpiperidine (4.4 ml, 26 mmol) was added to a cooled (−78° C.) solution of n-butyl lithium (9.4 ml, 2.5M in hexane, 23.5 mmol) in tetrahydrofuran (50 ml), and the solution stirred for 30 minutes. 2-Chloro-5-methylpyridine (3 g, 23.5 mmol) was then added, and the reaction stirred at −78° C. for 2.5 hours. The solution was poured onto solid carbon dioxide, and warmed to room temperature using a water bath. The solution was extracted with water, the aqueous acidified using 2N HCl, and extracted with ether. These organic extracts were washed with water and brine, then dried (MgSO₄) and evaporated under reduced pressure to afford the title compound as a yellow solid, 1.65 g.

¹HNMR (CDCl₃, 400 MHz) δ: 2.41 (s, 3H), 8.16 (s, 1H), 8.41 (s, 1H)

LRMS: m/z APCI⁺ 172 [MH]⁺

### PREPARATION 9

### Syn-{4-[(2-Chloro-5-methyl-pyridine-3-carbonyl)-amino]-cyclohexyl}-carbamic acid tert-butyl ester

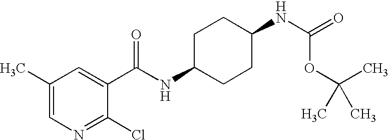

The title compound was obtained as a white foam in 82% yield from the nicotinic acid from preparation 8 and the amine from preparation 5, following the method of preparation 7.

¹HNMR (CDCl₃, 400 MHz) δ: 1.45 (s, 9H), 1.68-1.88 (m, 8H), 2.38 (s, 3H), 3.62 (m, 1H), 4.08 (m, 1H), 4.52 (m, 1H), 6.55 (m, 1H), 7.97 (s, 1H), 8.27 (s, 1H)

LRMS: m/z APCI⁺ 312 [MH₂-Bu]⁺

### PREPARATION 10

### Syn-{4-[(2-Chloro-pyridine-3-carbonyl)-amino]-cyclohexyl}-carbamic acid tert-butyl ester

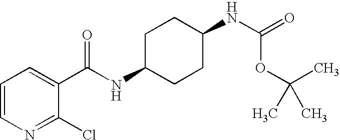

The title compound was obtained in 97% yield from 2-chloronicotinic acid and the amine from preparation 5, following the procedure described in preparation 6.

¹HNMR (CDCl₃, 400 MHz) δ: 1.33-1.49 (brs, 9H), 1.52-1.94 (m, 8H), 3.63 (m, 1H), 4.17 (brs, 1H), 4.53 (brs, 1H), 6.57 (brs, 1H), 7.38 (m, 1H), 8.16 (m, 1H), 8.48 (d, 1H)

LRMS: m/z ES⁺ 376 [MNa]⁺

### PREPARATION 11

### Syn-(4-{[5-fluoro-2-(tetrahydrothiopyran-4-yloxy)-pyridine-3-carbonyl]-amino}-cyclohexyl)-carbamic acid tert-butyl ester

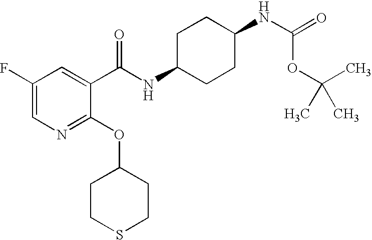

A mixture of the chloride from preparation 6 (3 g, 8.1 mmol), tetrahydrothiopyran-4-ol (WO 94/14793, pg 77) (2.4 g, 20.3 mmol) and cesium carbonate (6.5 g, 20 mmol) in acetonitrile (15 ml) was stirred at 100° C. for 24 hours. The cooled mixture was partitioned between water and ethyl acetate and the layers separated. The organic phase was washed with 10% citric acid solution, saturated sodium bicarbonate solution, water and brine, then dried (MgSO₄) and evaporated under reduced pressure to afford the title compound, 4.1 g.

¹HNMR (CDCl₃, 400 MHz) δ: 1.44-1.49 (s, 9H), 1.50-1.77 (m, 4H), 1.79-1.99 (m, 4H), 2.42 (m, 2H), 2.81 (m, 4H), 3.65 (m, 1H), 4.12 (m, 1H), 4.55 (m, 1H), 5.32 (m, 1H), 8.03 (m, 2H), 8.26 (m, 1H),

LRMS: m/z ACPI⁺ 476 [MNa]⁺

### PREPARATION 12

### Syn-(4-{[5-Chloro-2-(tetrahydrothiopyran-4-yloxy)-pyridine-3-carbonyl]-amino}-cyclohexyl)-carbamic acid tert-butyl ester

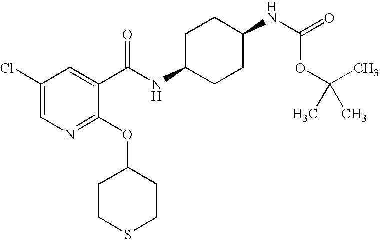

A mixture of the chloride from preparation 7 (1 g, 2.57 mmol), tetrahydrothiopyran-4-ol (WO 94/14793, pg 77) (500 mg, 4.23 mmol) and cesium carbonate (1.4 g, 4.23 mmol) in acetonitrile (5 ml) was stirred at reflux for 20 hours. The cooled mixture was partitioned between water (75 ml) and ethyl acetate (75 ml) and the layers separated. The organic phase was washed with water, 1N HCl, saturated sodium bicarbonate solution and brine, then dried (MgSO₄) and evaporated under reduced pressure. The product was purified by column chromatography on silica gel using an elution gradient of ethyl acetate:pentane (5:95 to 70:30) to afford the title compound as a white solid, 1.02 g.

¹HNMR (CDCl₃, 400 MHz) δ: 1.45 (s, 9H), 1.49-2.00 (m, 10H), 2.41 (m, 2H), 2.79 (m, 4H), 3.67 (m, 1H), 4.13 (m, 1H), 4.60 (m, 1H), 5.34 (m, 1H), 7.91 (m, 1H), 8.14 (d, 1H), 8.47 (d, 1H)

LRMS: m/z APCI⁺ 470 [MH]+

### PREPARATION 13

### Syn-(4-{[5-Methyl-2-(tetrahydrothiopyran-4-yloxy)-pyridine-3-carbonyl]-amino}-cyclohexyl)-carbamic acid tert-butyl ester

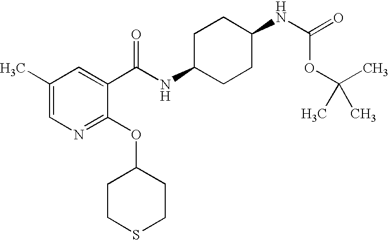

The title compound was obtained in 67% yield from the chloride from preparation 9 and tetrahydrothiopyran-4-ol (WO 94/14793, pg 77), following the procedure described in preparation 12.

¹HNMR (CDCl₃, 400 MHz) δ: 1.45 (s, 9H), 1.62-1.75 (m, 4H), 1.80-1.97 (m, 6H), 2.27 (s, 3H), 2.41 (m, 2H), 2.80 (m, 4H), 3.63 (m, 1H), 4.11 (m, 1H), 4.60 (m, 1H), 5.37 (m, 1H), 8.01 (s, 2H), 8.35 (m, 1H),

LRMS: m/z APCI⁺ 450 [MH]⁺

### PREPARATION 14

### Syn-(4-{[2-(Tetrahydrothiopyran-4-yloxy)-pyridine-3-carbonyl]-amino}-cyclohexyl)-carbamic acid tert-butyl ester

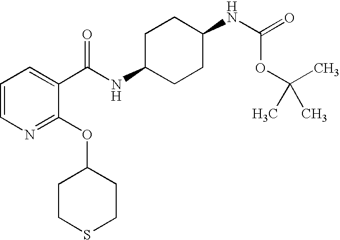

The title compound was obtained in 84% yield from the chloride from preparation 10 and tetrahydrothiopyran-4-ol (WO 94/14793, pg 77), following the procedure described in preparation 12.

¹HNMR (CDCl₃, 400 MHz) δ: 1.37-1.50 (s, 9H), 1.52-2.91 (m, 16H), 3.64 (m, 1H), 4.12 (m, 1H), 4.58 (brs, 1H), 5.41 (m, 1H), 7.04 (m, 1H), 7.98 (d, 1H), 8.22 (m, 1H), 8.53 (d, 1H)

LRMS: m/z ES⁺ 436 [MH]⁺, 458 [MNa]³⁰

### PREPARATION 15a

### Syn-N-(4-Amino-cyclohexyl)-5-fluoro-2-(tetrahydrothiopyran-4-yloxy)-nicotinamide hydrochloride

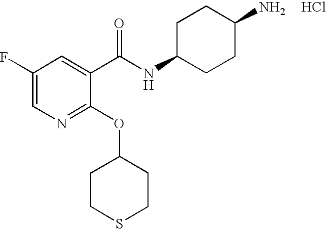

4N Hydrochloric acid in dioxan (50 ml) was added to a solution of the protected amine from preparation 11 (4.1 g, 9.0 mmol) in dichloromethane (10 ml), and the reaction stirred at room temperature for 3 hours. The mixture was evaporated under reduced pressure, the residue suspended in ether and the suspension sonicated. The mixture was filtered, the solid dried at 50° C. in vacuo to give the title compound as an off-white solid, 2.8 g.

¹HNMR (CD₃OD, 400 MHz) δ: 1.64-2.02 (m, 10H), 2.42 (m, 2H), 2.78 (m, 4H), 3.30 (m, 1H), 4.10 (m, 1H), 5.30 (m, 1H), 8.04 (m, 1H), 8.18 (d, 1H)

LRMS: m/z ES⁺ 354 [MH]⁺

### PREPARATION 15b

### Syn-N-(4-Amino-cyclohexyl)-5-fluoro-2-(tetrahydrothiopyran-4-yloxy)-nicotinamide

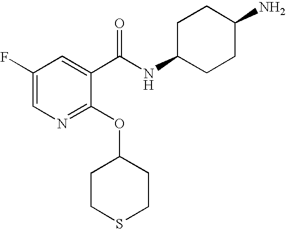

The amine hydrochloride from preparation 15a (95 mg, 0.24 mmol) was partitioned between dichloromethane and 1N sodium hydroxide solution, and the layers separated. The aqueous phase was extracted further with dichloromethane (2×), and the combined organic solutions dried (MgSO₄) and evaporated under reduced pressure to give the title compound, 75 mg.

¹HNMR (CDCl₃, 400 MHz)δ: 1.56-2.06 (m, 12H), 2.44 (m, 2H), 2.78 (m, 4H), 2.98 (m, 1H), 4.16 (m, 1H), 5.28 (m, 1H), 8.04 (m, 2H), 8.24 (m, 1H)

### PREPARATION 16

### Syn-N-(4-Amino-cyclohexyl)-5-chloro-2-(tetrahydrothiopyran-4-yloxy)-nicotinamide hydrochloride

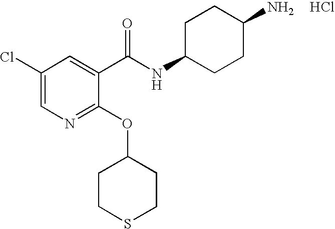

4N Hydrochloric acid in dioxan (15 ml) was added to a solution of the compound from preparation 12 (980 mg, 2.1 mmol) in dichloromethane (5 ml), and the reaction stirred at room temperature for 3 hours. Diisopropyl ether was added, the resulting suspension filtered, and the solid washed with further diisopropyl ether and dried in vacuo to afford the title compound, 835 mg.

¹HNMR (DMSO-d₆, 400 MHz) δ: 1.57-1.93 (m, 10H), 2.22-2.30 (m, 2H), 2.61-2.78 (m, 4H), 3.15 (m, 1H), 3.92 (m, 1H), 5.17 (m, 1H), 7.92-8.12 (m, 5H), 8.32 (s, 1H)

LRMS: m/z APCI⁺ 370 [MH]⁺

Microanalysis found; C, 49.92; H, 6.29; N, 10.09. C₁₇H₂₄ClN₃O₂S; HCl; 0.15H₂O requires C, 49.91; H, 6.23; N, 10.27%.

### PREPARATION 17

### Syn-N-(4-Amino-cyclohexyl)-5-methyl-2-(tetrahydrothiopyran-4-yloxy)-nicotinamide hydrochloride

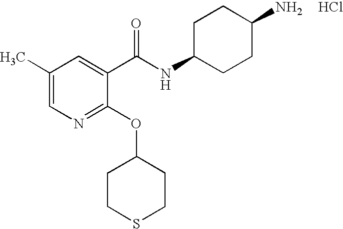

4N Hydrochloric acid in dioxan (15 ml) was added to a solution of the protected amine from preparation 13 (800 mg, 1.78 mmol) In dichloromethane (5 ml), and the reaction stirred at room temperature for 3 hours. The mixture was evaporated under reduced pressure, the residue suspended in diisopropyl ether and the suspension sonicated. The mixture was filtered, and the solid dried at 50° C. in vacuo to give the title compound as an off-white solid, 457 mg.

¹HNMR (DMSO-d₆, 400 MHz) δ: 1.57-1.92 (m, 10H), 2.22-2.34 (m, 5H), 2.64-2.77 (m, 4H), 3.15 (m, 1H), 3.91 (m, 1H), 5.17 (m, 1H), 7.89-8.07 (brs, 4H), 8.11 (s, 1H)

LRMS: m/z APCI⁺ 350 [MH]⁺

Microanalysis found; C, 55.52; H. 7.43; N, 10.38. C₁₈H₂₇FN₃O₂S; HCl; 0.33H₂O requires C, 55.16; H, 7.37; N, 10.72%.

### PREPARATION 18

### Syn-N-(4-Amino-cyclohexyl)-2-(tetrahydrothiopyran-4-yloxy)-nicotinamide hydrochloride

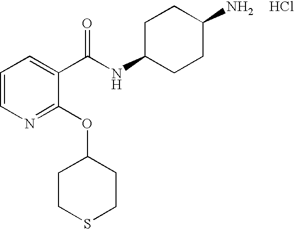

The title compound was obtained in 96% yield from the compound from preparation 14, following the procedure described in preparation 15a.

¹HNMR (DMSO-d₆, 400 MHz) δ: 1.54-2.00 (m, 8H), 2.27-2.39 (m, 2H), 2.50 (s, 2H), 2.59-2.80 (m, 4H), 3.14 (brs, 1H), 3.92 (brs, 1H), 5.22 (m, 1H), 7.10 (q, 1H), 8.01 (d, 1H), 8.11 (m, 2H), 8.27 (m, 1H)

LRMS: m/z ES⁺ 336 [MH]⁺

### PREPARATION 19

### 2-Amino-1-(3-ethoxy-2,3-dioxopropyl)pyridinium bromide

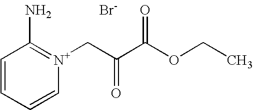

Ethyl bromopyruvate (51.9 g, 266 mmol) was added dropwise to a solution of 2-aminopyridine (25 g, 266 mmol) in ethylene glycol dimethyl ether (270 ml), and the reaction then stirred at room temperature for 1 hour. The resulting precipitate was filtered off, the solid washed with ether and dried to afford the title compound as a pale yellow solid, 71.9 g.

¹HNMR (CDCl₃, 300 MHz) δ: 1.35 (t, 3H), 4.35 (q, 2H), 4.70 (d, 1H), 5.15 (d, 1H), 7.10-7.20 (m, 2H), 8.10 (m, 1H), 8.25 (d, 1H).

### PREPARATION 20

### Ethyl imidazo[1,2-a]pyridine-2-carboxylate hydrobromide

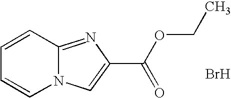

A suspension of the compound from preparation 19 (71.9 g, 249 mmol) in ethanol (750 ml) was heated at reflux for 3 hours, then allowed to cool. The mixture was concentrated under reduced pressure, the residue triturated with ether; filtered and dried to afford the title compound as a solid, 64.17 g.

¹HNMR (CD₃OD, 300 MHz) δ: 1.45 (t, 3H), 4.50 (q, 2H), 7.55 (m, 1H), 7.95 (m, 1H), 8.10 (dd, 1H), 8.80 (s, 1H), 8.85 (d, 1H).

### PREPARATION 21

### Imidazo[1,2-a]pyridine-2-carboxylic acid hydrobromide

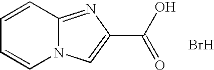

A solution of the ester from preparation 20 (5.0 g, 18.4 mmol) in 10% aqueous hydrobromic acid (90 ml) was heated under reflux for 6 hours. The cooled mixture was concentrated under-reduced pressure and the residue triturated with dioxan. The resulting solid was filtered off, washing with hexane and the filtrate evaporated under reduced pressure. The residue was again triturated with dioxan, the solid filtered and dried to afford additional compound, 3.83 g in total.

¹HNMR (CD₃OD, 300 MHz) δ: 7.57 (m, 1H), 7.96 (d, 1H), 8.06 (m, 1H), 8.78 (s, 1H), 8.84 (d, 1H).

LRMS: m/z ES⁺ 163 [MH]⁺

### PREPARATION 22

### Methyl imidazo[1,2-a]pyridine-8-carboxylate

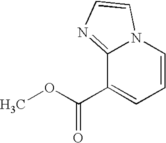

A mixture of methyl 2-aminonicotinate (WO 89/01488 pg 33, prep 17) (1 g, 6.56 mmol), and chloroacetaldehyde (1.05 ml, 6.56 mmol) in ethanol (5 ml) was heated under reflux for 18 hours. The cooled mixture was diluted with water (10 ml), 0.88 ammonia (1 ml) added and the solution concentrated under reduced pressure. The residue was dissolved in methanol and the dark solution treated with charcoal, the mixture filtered and the filtrate evaporated under reduced pressure. The residue was purified by column chromatography on silica gel using dichloromethane:methanol:0.88 ammonia (97:2.5:0.5) as eluant, and the product triturated with ether, to afford the title compound, 768 mg.

¹HNMR (CDCl₃, 400 MHz) δ: 4.02 (s, 3H), 6.83 (s, 1H), 7.63 (s, 1H), 7.79 (s, 1H), 8.00 (d, 1H), 8.31 (d, 1H).

LRMS: m/z TSP⁺ 177.2 ([MH⁺]

### PREPARATION 23

### Imidazo[1,2-a]pyridine-8-carboxylic acid

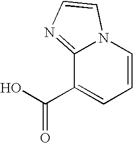

Lithium hydroxide solution (2.5 ml, 1M in water) was added to a solution of the ester from preparation 22 (400 mg, 2.27 mmol) in methanol (5 ml) and the solution stirred at room temperature for 90 minutes. The solution was concentrated under reduced pressure to remove the methanol, the aqueous solution acidified using 2N hydrochloric acid, and the mixture evaporated under reduced pressure to give the title compound as a yellow solid.

¹HNMR (DMSO-d₆, 400 MHz) δ: 7.60 (dd, 1H), 8.10 (s, 1H), 8.41 (d, 1H), 8.55 (s, 1H), 9.18 (d, 1H)

LRMS: m/z TSP⁺ 163 [MH]⁺

### PREPARATION 24

### 7-Methoxy-imidazo[1,2-a]Pyridine-8-carbonitrile

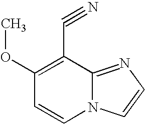

A mixture of 2-amino-4-methoxynicotinonitrile (Archiv. Der. Pharmazie 318(6); 1985; 481) (19, 6.5 mmol), and chloroacetaldehyde (1.25 g, 8 mmol) in ethanol (10 ml) was stirred at room temperature for 1 hour, then heated at reflux for 18 hours. The cooled mixture was basified by the addition of saturated sodium bicarbonate solution, and the resulting solid filtered off, washed with water and dried in vacuo to afford the title compound, 1 g.

¹HNMR (DMSO-de, 400 MHz) δ: 4.03 (s, 3H), 7.11 (d, 1H), 7.51 (s, 1H), 7.91 (s, 1H), 8.82 (d, 1H).

LRMS: m/z APCI⁺ 174 [MH]⁺

### PREPARATION 25

### 7-Methoxy-imidazo[1,2-a]pyridine-8-carboxylic acid

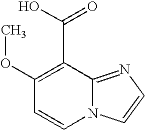

A solution of the nitrile from preparation 24 (600 mg, 3.47 mmol) in sulphuric acid (3 ml) and water (3 ml) was stirred at 60° C. for 24 hours. The cooled solution was diluted with ether (20 ml), then ethanol added until precipitation occurred. The resulting solid was filtered off, washed with ethanol and ether and dried in vacuo. The solid was dissolved in 6N hydrochloric acid, the solution stirred at 90° C. for 5 hours, and concentrated under reduced pressure to afford the title compound, 110 mg.

LRMS: m/z APCI⁺ 193 [MH]⁺

### PREPARATION 26

### Ethyl 3-hydroxymethyl-imidazo[1,2-a]pyridine-8-carboxylate

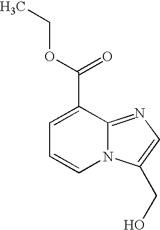

A mixture of 8-carboethoxyimidazo[1,2-a]pyridine (U.S. Pat. No. 5,294,612, ex 114(b)) (655 mg, 3.45 mmol), sodium acetate (1.06 g, 13 mmol), formaldehyde (37% aq solution, 1.8 ml, 22 mmol) and acetic acid (0.75 ml) was stirred at room temperature for 4 hours, then heated at reflux for 6 hours. The cooled mixture was dissolved in water (20 ml), potassium carbonate added, to achieve pH 8, and the solution extracted with ethyl acetate (3×25 ml). The combined organic solutions were washed with saturated sodium bicarbonate solution and brine, then dried (MgSO₄) and concentrated under reduced pressure. The crude product was purified by column chromatography on silica gel using an elution gradient of dichloromethane:methanol:0.88 ammonia (99.5:0.5:0 to 94:6:0.6) to afford the title compound.

¹HNMR (CDCl₃, 400 MHz) δ: 1.45 (t, 3H), 2.47 (brs, 1H), 4.51 (q, 2H), 4.94 (s, 2H), 6.93 (m, 1H), 7.40 (s, 1H), 8.00 (d, 1H), 8.51 (d, 1H).

### PREPARATION 27

### 3-Hydroxymethyl-imidazo[1,2-a]pyridine-8-carboxylic acid

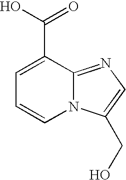

A solution of the ester from preparation 26 (200 mg, 0.9 mmol), 1N sodium hydroxide (1 ml) and methanol (5 ml) was stirred at room temperature for 18 hours. The solution was acidified using 2N hydrochloric acid (2 ml) and evaporated under reduced pressure.

¹HNMR (CD₃OD, 400 MHz) δ: 5.06 (s, 2H), 7.67 (m, 1H), 8.03 (s, 1H), 8.66 (d, 1H), 9.04 (d, 1H).

LRMS: m/z ES⁺ 193 [MH]⁺

### PREPARATION 28

### 1H-Benzoimidazole-4-carboxylic acid

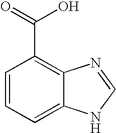

A suspension of 3-nitroanthranillic acid (J. Chem. Soc. 127; 1925; 1791) (4.0 g, 22 mmol) and palladium on charcoal (400 mg) in ethanol was hydrogenated at room temperature using a Parr shaker for 4 hours. The mixture was filtered and the filtrate acidified with conc. hydrochloric acid. Formic acid (2.49 ml, 65.9 mmol) was added and the solution heated under reflux for 2 hours. The solution was concentrated under reduced pressure to 7 low volume, cooled In ice, and the resulting precipitate filtered. Further precipitation of the filtrate occurred, this solid was filtered and the combined products were recrystallised from 0.5N hydrochloric acid to give the title compound, 2.62 g.

LRMS: m/z 162.1 [MH⁺]

### PREPARATION 29

### Ethyl 2-amino-3-isopropylamino-benzoate

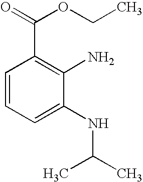

2-Iodopropane (2.0 ml, 20 mmol) was added to a solution of ethyl 2,3-diaminobenzoate (WO 97/10219 ex51(1)) (3 g, 16.67 mmol) in N,N-dimethylformamide (20 ml), and the solution stirred at 50° C. for 3 hours. The mixture was concentrated under reduced pressure and the residue partitioned between ethyl acetate (200 ml) and water (50 ml), and the layers separated. The organic layer was washed with water (5×50 ml), dried (MgSO₄) and evaporated under reduced pressure. The crude product was purified by column chromatography on silica gel using ethyl acetate:pentane (5:95 to 90:10) to afford the title compound as a yellow oil, 1.4 g.

¹HNMR (CDCl₃, 400 MHz) δ: 1.20 (d, 6H), 1.38 (t, 3H), 3.56 (m, 1H), 4.31 (q, 2H), 5.60 (brs, 2H), 6.84 (m, 1H), 6.80 (d, 1H), 7.42 (d, 1H).

LRMS: m/z ES⁺ 223 [MH]⁺

### PREPARATION 30

### 1-Isopropyl-1H-benzoimidazole-4-carboxylic acid

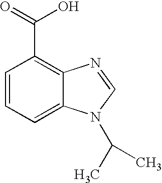

A solution of the amine from preparation 29 (1.4 g, 6.31 mmol) in formic acid (15 ml) was stirred at 60° C. for 45 minutes. 2M Hydrochloric acid (20 ml) and additional formic acid (15 ml) was added and the reaction heated under reflux for 12 hours. The cooled mixture was evaporated under reduced pressure and the residue triturated initially with ethyl acetate and the solid filtered and dried. This solid was then triturated with hot ethyl acetate and the solid filtered and dried at 60° C. to give the title compound as a pale pink solid, 1.16 g.

¹HNMR (DMSO-d₆, 400 MHz) δ: 1.61 (d, 6H), 5.10 (m, 1H), 7.72 (m, 1H), 8.13 (d, 1H), 8.39 (d, 1H), 9.75 (s, 1H).

LRMS: m/z TSP⁺ 205 [MH]⁺

### PREPARATION 31

### Ethyl 1-[2-(Tetrahydropyran-2-yloxy)-ethyl]-1H-indazole-3-carboxylate

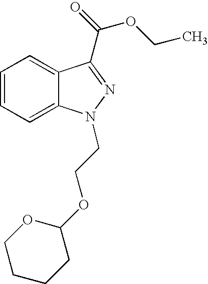

A mixture of indazole-3-carboxylic acid ethyl ester (Chem. Ber. 52; 1919; 1345) (1.9 g, 10 mmol), 2-(2-bromoethyloxy)tetrahydropyran (2.25 g, 10.8 mmol), potassium carbonate (1.43 g, 10.4 mmol) and lithium iodide (67 mg, 0.5 mmol) in 1-methyl-2-pyrrolidinone (20 ml) was heated at 80° C. for 17 hours. The mixture was partitioned between water (250 ml) and ethyl acetate (250 ml), and the layers separated. The organic phase was washed with water (3×200 ml), dried (MgSO₄) and evaporated under reduced pressure. The residual oil was purified by column chromatography on silica gel using an elution gradient of pentane:ethyl acetate (91:9 to 50:50) to afford the title compound as a pale yellow oil, 1.88 g.

¹HNMR (DMSOd₆, 400 MHz) δ: 1.20-1.53 (m, 6H), 1.35 (t, 3H), 3.30 (m, 2H), 3.80 (m, 1H), 4.00 (m, 1H), 4.37 (q, 2H), 4.48 (m, 1H), 4.70 (m, 2H), 7.32 (m, 1H), 7.80 (d, 1H), 8.05 (d, 1H).

LRMS: m/z ES⁺ 341 [MNa⁺]

### PREPARATION 32

### 1-[2-(Tetrahydropyran-2-yloxy)-ethyl]-1H-indazole-3-carboxylic acid

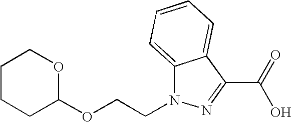

A solution of sodium hydroxide (413 mg, 10.3 mmol) in water (3.75 ml) was added dropwise to a solution of the ester from preparation 31 (1.83 g, 5.74 mmol) in ethanol (14.7 ml), and the reaction stirred at room temperature for 2 days. The mixture was acidified to pH 3 using 2N hydrochloric acid, and the mixture partitioned between ethyl acetate (75 ml) and water (75 ml). The layers were separated, and the aqueous further extracted with ethyl acetate (3×60 ml). The combined organic solutions were dried (MgSO₄) and evaporated under reduced pressure to give the title compound as a white crystalline solid, 1.44 g.

¹HNMR (DMSOd₆, 400 MHz) δ: 1.20-1.55 (m, 6H), 3.30 (m, 2H), 3.80 (m, 1H), 4.00 (m, 1H), 4.48 (m, 1H), 4.68 (m, 2H), 7.28 (m, 1H), 7.46 (m, 1H), 7.80 (d, 1H), 8.08 (d, 1H), 12.90 (brs, 1H).

LRMS: m/z ES-289 [M-H⁻]

### PREPARATION 33

### Ethyl 5-methyl-1-[2-(tetrahydropyran-2-yloxy)ethyl]-1H-pyrazole-3-carboxylate

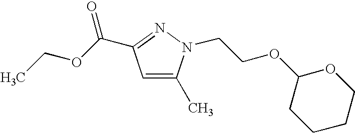

Ethyl 3-methylpyrazole-5-carboxylate (3 g, 19.5 mmol) was added to a suspension of sodium hydride (934 mg, 60% dispersion in mineral oil, 23.35 mmol) in tetrahydrofuran (50 ml), and the solution stirred at room temperature for 30 minutes. 2-(2-Bromoethoxy)tetrahydro-2-pyran (3.5 ml, 23.35 mmol) and lithium iodide (50 mg, 0.37 mmol) were added and the reaction heated under reflux for 16 hours. The cooled mixture was partitioned between water and ethyl acetate and the layers separated. The organic phase was washed sequentially with 10% citric acid, water, saturated sodium bicarbonate solution, water then brine, dried (MgSO₄) and concentrated under reduced pressure. The crude product was purified by column chromatography on silica gel using an elution gradient of methanol:dichloromethane (1:99 to 5:95) to afford the title compound, 4.47 g.

¹HNMR (CDCl₃, 400 MHz) δ: 1.38 (t, 3H), 1.40-1.76 (m, 6H), 2.36 (s, 1H), 3.41 (m, 1H), 3.59 (m, 1H), 3.76 (m, 1H), 4.06 (m, 1H), 4.32 (t, 2H), 4.37 (q, 2H), 4.47 (m, 1H), 6.53 (s, 1H)

LRMS: m/z ES⁺ 305 [MNa]⁺

### PREPARATIONS 34 and 35

### Ethyl 5-isopropyl-2-[2-(tetrahydro-pyran-2-yloxy)ethyl]-2H-pyrazole-3-carboxylate And Ethyl 5-Isopropyl-1-[2-(tetrahydro-pyran-2-yloxy)-ethyl]-1H-pyrazole-3-carboxylate

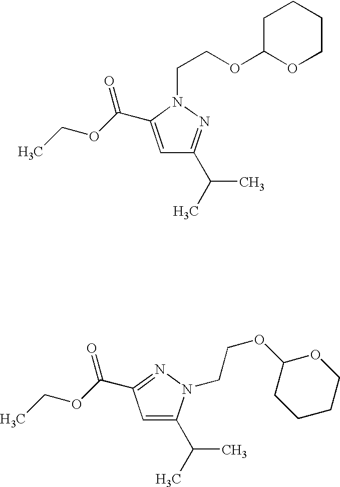

A mixture of ethyl 5-isopropyl-1H-pyrazole-3-carboxylate (Chem. and Pharm. Bull. 1984; 32(4); 1568) (509 mg, 2.8 mmol), 2-(2-bromoethoxy)tetrahydro-2-pyran (732 mg, 3.5 mmol) and potassium carbonate (483 mg, 3.5 mmol) in 1-methyl-2-pyrrolidinone (5 ml) was stirred at 80° C. for 18 hours. The cooled mixture was poured Into ethyl acetate and washed with water and brine, then dried (MgSO₄) and evaporated under reduced pressure. The residue was purified by column chromatography on silica gel using an elution gradient of ethyl acetate:pentane (20:80 to 40:60) to afford the title compound of preparation 34 as a clear oil, 663 mg.

¹HNMR (CDCl₃, 400 MHz) δ: 1.25 (d, 6H), 1.37 (t, 3H), 1.44-1.71 (m, 6H), 2.97 (m, 1H), 3.42 (m, 1H), 3.54 (m, 1H), 3.75 (m, 1H), 4.00 (m, 1H), 4.32 (q, 2H), 4.54 (t, 1H), 4.68 (m, 1H), 4.76 (m, 1H), 6.64 (s, 1H).

LRMS: m/z ACPI⁺ 311 [MH]⁺

Further elution provided the title compound of preparation 35, 242 mg.

¹HNMR (CDCl₃, 400 MHz) δ: 1.25 (d, 6H), 1.38 (t, 3H), 1.46-1.72 (m, 6H), 3.15 (m, 1H), 3.45 (m, 1H), 3.65 (m, 1H), 3.81 (m, 1H), 4.10 (m, 1H), 4.34 (m, 2H), 4.39 (m, 2H), 4.49 (t, 1H), 6.57 (s, 1H).

LRMS: m/z ACPI⁺311 [MH]⁺

### PREPARATION 36

### Methyl 5-ethyl-2H-pyrazole-3-carboxylate

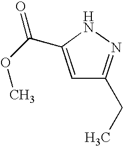

The title compound was prepared by analogy with the method described in Chem. and Pharm. Bull. 1984; 32(4);1568.

¹HNMR (DMSO-d₆, 400 MHz) δ: 1.20 (t, 3H), 2.60 (q, 2H), 3.60 (s, 3H), 6.50 (s, 1H).

LRMS: m/z APCI⁺ 155 [MH]⁺

### PREPARATION 37 and 38

### Methyl 3-ethyl-1-[2-(tetrahydro-2H-pyran-2-yloxy)ethyl]-1H-pyrazole-5-carboxylate And Methyl 5-ethyl-1-[2-(tetrahydro-2H-pyran-2-yloxy)ethyl]-1H-pyrazole-3-carboxylate

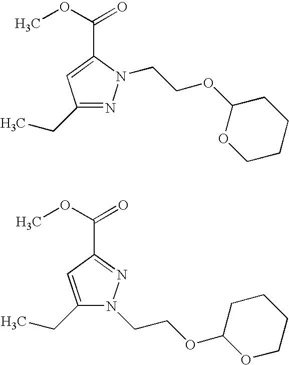

The title compounds were prepared from the ester from preparation 36 and 2-(2-bromoethoxy)tetrahydro-2-pyran by analogy with the method described for preparations 34 and 35.

### PREPARATION 37

¹HNMR (CDCl₃, 400 MHz) δ: 1.16 (t, 3H), 1.38-1.75 (m, 6H), 2.48 (m, 2H), 3:35 (m, 1H), 3.54 (m, 1H), 3.70 (m, 1H), 3.80 (s, 3H), 3.95 (m, 1H), 4.50 (m, 1H), 4.68 (m, 2H), 6.60 (s, 1H).

### PREPARATION 38

¹HNMR (CDCl₃, 400 MHz) δ: 1.25 (t, 3H), 1.38-1.68 (m, 6H), 2.70 (t, 2H), 3.38 (m, 1H), 3.54 (m, 1H), 3.72 (m, 1H), 3.85 (s, 3H), 4.04 (m, 1H), 4.25 (m, 2H), 4.43 (m, 1H), 6.54 (s, 1H).

### PREPARATION 39

### 5-Methyl-1-[2-(tetrahydro-pyran-2-yloxy)ethyl]-1H-Pyrazole-3-carboxylic acid

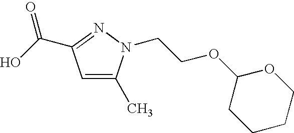

A mixture of the ester from preparation 33 (3 g, 10.6 mmol) and lithium hydroxide solution (50 ml, 50 mmol) in tetrahydrofuran (50 ml) was stirred at room temperature for 24 hours. The mixture was diluted with ethyl acetate and the layers separated. The aqueous phase was acidified using 2N hydrochloric acid, and extracted with ethyl acetate. These combined organic extracts were washed with water, brine, then dried (MgSO₄) and evaporated under reduced pressure to afford the title compound, 1.8 g.

¹HNMR (CDCl₃, 400 MHz) δ: 1.42-1.75 (m, 6H), 2.37 (s, 3H), 3.33 (m, 1H), 3.58 (m, 1H), 3.78 (m, 1H), 4.11 (m, 1H), 4.35 (t, 2H), 4.50 (m, 1H), 6.59 (s, 1H)

LRMS: m/z ACPI⁻ 253 [M-H]⁻

### PREPARATION 40

### 3-Ethyl-1-[2-(tetrahydro-2H-pyran-2-yloxy)ethyl]-1H-pyrazole-5-carboxylic acid

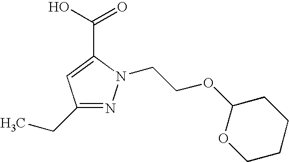

The title compound was prepared as a solid from the ester from preparation 37, following the procedure described in preparation 39.

¹HNMR (CDCl₃, 400 MHz) δ: 1.24 (t, 3H), 1.41-1.85 (m, 5H), 2.64 (q, 2H), 3.42 (m, 1H), 3.60 (m, 1H), 3.76 (m, 2H), 4.02 (m, 1H), 4.57 (m, 1H), 4.65-4.81 (m, 2H), 6.73 (s, 1H)

LRMS: m/z ES⁺ 291 [MNa]⁺

### PREPARATION 41

### 5-Ethyl-1-[2-(tetrahydro-2H-pyran-2-yloxy)ethyl]-1H-pyrazole-3-carboxylic acid

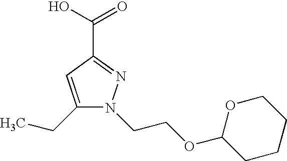

The title compound was prepared as a solid from the ester from preparation 38, following the procedure described in preparation 39.

¹HNMR (CDCl₃, 400 MHz) δ: 1.27 (t, 3H), 1.41-1.89 (m, 6H), 2.71 (q, 2H), 3.38-3.64 (m, 2H), 3.7-3.85 (m, 1H), 3.97-4.12 (m, 1H), 4.30 (t, 2H), 4.48 (s, 1H), 4.93 (s, 1H), 6.73 (s, 1H)

LRMS: m/z ES⁺ 291 [MNa]⁺

### PREPARATION 42

### 5-isopropyl-2-[2-(tetrahydro-pyran-2-yloxy)ethyl]-2H-pyrazole-3-carboxylic acid

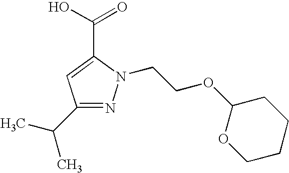

A mixture of the ester from preparation 34 (660 mg, 2.13 mmol), and 2M sodium hydroxide (2.5 ml, 5 mmol) in ethanol (10 ml) was stirred at room temperature for 18 hours. The reaction mixture was diluted with ethyl acetate, washed with 0.5N citric add and brine, dried (MgSO₄) and evaporated under reduced pressure to give the title compound as a clear oil, 570 mg.

¹HNMR (CDCl₃, 400 MHz) δ: 1.26 (d, 6H), 1.43-1.72 (m, 6H), 3.00 (m, 1H), 3.42 (m, 1H), 3.54 (m, 1H), 3.77 (m, 1H), 4.02 (m, 1H), 4.56 (m, 1H), 4.74 (m, 2H), 6.75 (s, 1H).

LRMS: m/z APCI⁺ 283 [MH]⁺

### PREPARATION 43

### 5-Isopropyl-1-[2-(tetrahydro-pyran-2-yloxy)-ethyl]-1H-pyrazole-3-carboxylic acid

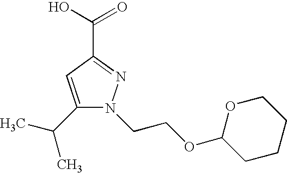

The title compound was obtained quantitatively from the ester from preparation 35 following the procedure described in preparation 42.

¹HNMR (CDCl₃, 400 MHz) δ: 1.28 (d, 6H), 1.46-1.71 (m, 6H), 3.15 (m, 1H), 3.45 (m, 1H), 3.62 (m, 1H), 3.83 (m, 1H), 4.12 (m, 1H), 4.34 (m, 2H), 4.57 (m, 1H), 6.63 (s, 1H).

LRMS: m/z APCI⁻ 281 [M-H]⁻

### PREPARATION 44

### Methyl 3-ethanesulphonylamino-benzoate

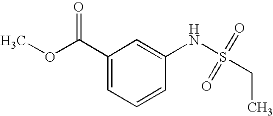

A solution of ethylsulphonyl chloride (1.25 ml, 13.2 mmol) in dichloromethane (10 ml) was added dropwise over 5 minutes to an ice-cooled solution of methyl 3-aminobenzoate (2 g, 13.2 mmol) and pyridine (1.6 ml, 19.8 mmol) in dichloromethane (30 ml). The reaction was stirred at room temperature for 18 hours, then partitioned between 2N hydrochloric acid and dichloromethane. The layers were separated, the aqueous phase extracted with dichloromethane and the combined organic solutions dried (MgSO₄) and evaporated under reduced pressure. The crude product was purified by column chromatography on silica gel using an elution gradient of dichloromethane:acetonitrile (99:1 to 90:10) to afford the title compound, 2.989.

¹HNMR (CDCl₃, 400 MHz) δ: 1.40 (t, 3H), 3.16 (q, 2H), 3.94 (s, 3H), 7.04 (s, 1H), 7.24 (m, 1H), 7.52 (m, 1H), 7.86 (m, 2H)

LRMS: m/z ES⁺ 266 [MNa]⁺

### PREPARATION 45

### Methyl 3-isopropylsulphonylamino-benzoate

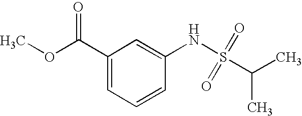

The title compound was obtained in 12% yield from methyl 3-aminobenzoate and isopropyl sulphonyl chloride following the procedure described in preparation 44.

¹HNMR (CDCl₃, 400 MHz) δ: 1.40 (d, 6H), 3.32 (m, 1H), 3.94 (s, 3H), 7.20 (m, 1H), 7.40 (m, 1H), 7.56 (m, 1H), 7.80 (m, 1H), 7.88 (s, 1H)

LRMS: m/z ES⁺ 280 [MNa]⁺

### PREPARATION 46

### Methyl 3-methylsulphonylamino-benzoate

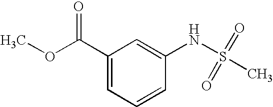

A solution of methane suphonyl chloride (1.03 ml, 13.2 mmol) in dichloromethane (10 ml) was added dropwise to an ice-cooled solution of methyl 3-aminobenzoate (2 g, 13.2 mmol) and triethylamine (3.68 ml, 26.4 mmol) in dichloromethane (40 ml), and the reaction stirred at room temperature for 18 hours. Additional triethylamine (1.84 ml, 13.2 mmol) and methane sulphonyl chloride (0.52 ml, 6.6 mmol) were added and the reaction stirred for a further 2 hours. The mixture was acidified carefully with 1N hydrochloric acid, then extracted with dichloromethane (3×). The combined organic extracts were dried (MgSO₄) and evaporated under reduced pressure. The crude product was purified by column chromatography on silica gel using dichloromethane:acetonitrile (99:1 to 94:6) to give the title compound, 1.5 g.

¹HNMR (CDCl₃, 400 MHz) δ: 3.04 (s, 3H), 3.94 (s, 3H), 6.84 (brs, 1H), 7.44-7.58 (m, 2H), 7.86 (m, 2H)

LRMS: m/z ES⁺ 252 [MNa]⁺

### PREPARATION 47

### Methyl 3-methanesulphonylmethylamino-benzoate

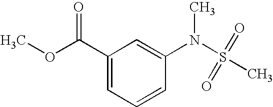

Sodium hydride (340 mg, 60% in mineral oil, 8.5 mmol) was added to an ice-cooled solution of the sulphonamide from preparation 46 (1.50 g, 6.5 mmol) in tetrahydrofuran (50 ml), and the solution stirred for 90 minutes. Methyl iodide (1.21 ml, 19.5 mmol) was added, and the reaction stirred at room temperature for 18 hours. The mixture was acidified using 1N hydrochloric acid, and extracted with ethyl acetate (2×). The combined organic extracts were dried (MgSO₄) and evaporated under reduced pressure. The crude product was purified by column chromatography on silica gel using an elution gradient of pentane:ethyl acetate:diethylamine (80:20:0.6 to 50:50:1) to afford the title compound as a white solid, 1.07 g.

¹HNMR (CDCl₃, 400 MHz) δ: 2.96 (s, 3H), 3.38 (s, 3H), 3.94 (s, 3H), 7.08 (t, 1H), 7.64 (m, 1H), 7.98 (m, 2H)

LRMS: m/z ES⁺ 266 [MNa]⁺

Microanalysis found; C, 49.48; H, 5.43; N, 5.78, C₁₀H₁₃NO₄S; requires C, 49.37; H. 5.39; N, 5.76%.

### PREPARATION 48

### 3-Methanesulphonylmethylamino-benzoic acid

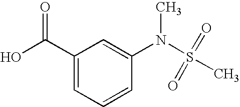

A solution of the ester from preparation 47 (1.05 g, 4.3 mmol), lithium hydroxide (43 ml, 1M, 43 mmol) in tetrahydrofuran (43 ml) was stirred at room temperature for 18 hours. The mixture was concentrated under reduced pressure to remove the tetrahydrofuran and the aqueous solution acidified using 2N hydrochloric acid. The resulting precipitate was filtered off, washed with water and dried in vacuo, to give the title compound, 773 mg.

¹HNMR (CD₃OD, 400 MHz) δ: 2.90 (s, 3H), 3.45 (s, 3H), 7.50 (m, 1H), 7.70 (d, 1H), 7.90 (d, 1H), 8.10 (s, 1H).

LRMS: m/z ES⁺ 252 [MNa]⁺

### PREPARATION 49

### 3-Ethanesulphonylamino-benzoic acid

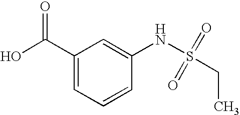

The title compound was obtained in 64% yield from the ester from preparation 44 following the procedure described in preparation 48.

¹HNMR (CD₃OD, 400 MHz) δ: 1.30 (s, 3H), 3.10 (q, 2H), 7.45 (m, 2H), 7.70 (d, 1H), 7.90 (d, 1H).

LRMS: m/z ES⁺ 252 [MNa]⁺

### PREPARATION 50

### 3-Isopropylsulphonylamino-benzoic acid

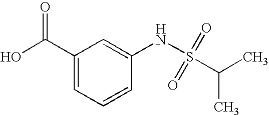

A solution of the ester from preparation 45 (398 mg, 1.55 mmol), and lithium hydroxide (15 ml, 1M, 15 mmol) in tetrahydrofuran (15 ml) was stirred at room temperature for 18 hours. The mixture was concentrated under reduced pressure to remove the tetrahydrofuran and the aqueous solution acidified using 2N hydrochloric acid. This solution was extracted with ethyl acetate (×3), the combined organic extracts washed with brine, dried (MgSO₄) and evaporated under reduced pressure to give the title compound, 376 mg.

¹HNMR (CD₃OD, 400 MHz) δ: 1.35 (d, 6H), 3.30 (m, 1H), 7.40 (m, 1H), 7.50 (d, 1H), 7.70 (d, 1H), 7.90 (s, 1H).

LRMS: m/z ES⁺ 242[MH]⁺

### PREPARATIONS 51 to 57

Potassium carbonate (2 eq) and potassium iodide (0.1 eq) were added to a solution of the appropriate phenol (1 eq) in acetonitrile (1.25 mlmmol⁻¹), and the mixture warmed to 90° C. 2-(2-Bromoethoxy)tetrahydro-2H-pyran (1.3 eq) was added and the reaction stirred at 90° C. for 72 hours. The cooled reaction was concentrated under reduced pressure and the residue partitioned between ethyl acetate and 10% citric acid solution, and the layers separated. The organic phase was washed with water, sodium bicarbonate solution and brine, then dried (MgSO₄) and evaporated under reduced pressure. The crude product was purified by column chromatography on silica gel using an elution gradient of ethyl acetate:pentane (5:95 to 50:50) to afford the title compounds as clear oils.

| Prep | | Yield | |
|---|---|---|---|
| No | | (%) | Data |
| 51¹ | 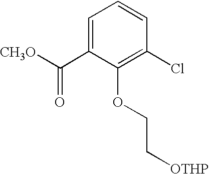 | 79 | ¹HNMR (CDCl₃, 400 MHz) δ: 1.50-1.90(m, 6H), 3.50 (m, 1H), 3.90 (m, 5H), 4.10(m, 1H), 4.20 (m, 2H), 4.70 (t, 1H), 7.10(m, 1H), 7.50 (dd, 1H), 7.70 (dd, 1H).LRMS: m/z ES⁺ 337 [MNa]⁺ |
| 52² | 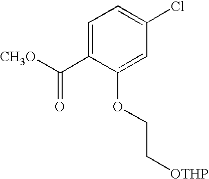 | 72 | ¹HNMR (CDCl₃, 400 MHz) δ: 1.44-1.90(m, 6H), 3.54 (m, 1H), 3.80-3.96 (m, 5H),4.06 (m, 1H), 4.24 (m, 2H), 4.74 (m, 1H),6.96 (m, 1H), 7.04 (d, 1H), 7.74(d, H)LRMS: m/z ES⁺ 337 [MNa]⁺ |
| 53³ | 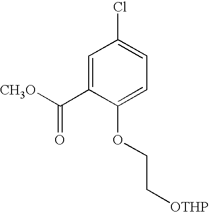 | 84 | ¹HNMR (CDCl₃, 400 MHz) δ: 1.46-1.90(m, 6H), 3.50 (m, 1H), 3.80-3.94 (m, 5H),4.06 (m, 1H), 4.20 (m, 2H), 4.74 (m, 1H),6.96 (d, 1H), 7.38 (m, 1H), 7.74 (d, 1H)LRMS: m/z ES⁺ 337 [MNa]⁺ |
| 54⁴ | 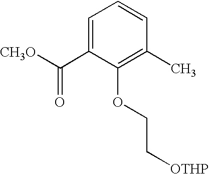 | 72 | ¹HNMR (CDCl₃, 400 MHz) δ: 1.44-1.94(m, 6H), 2.36 (s, 3H), 3.40-3.68 (m, 2H),3.70-3.96 (m, 5H), 4.10 (m, 2H), 4.74(m, 1H), 7.04 (m, 1H), 7.36 (m, 1H), 7.62(m, 1H)LRMS: m/z ES⁺ 317 [MNa]⁺ |
| 55⁵ | 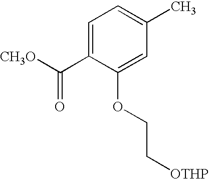 | 46 | ¹HNMR (CDCl₃, 400 MHz) δ: 1.48-1.92(m, 6H), 2.38 (m, 3H), 3.54 (m, 1H),3.80-3.94 (m, 5H), 4.06 (m, 1H), 4.22 (m,2H), 4.76 (m, 1H), 6.80 (m, 2H), 7.70 (d,1H)LRMS: m/z ES⁺ 317 [MNa]⁺ |
| 56⁶ | 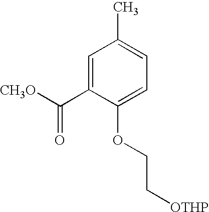 | 57 | ¹HNMR (CDCl₃, 400 MHz) δ: 1.44-1.90(m, 6H), 2.30 (m, 3H), 3.54 (m, 1H),3.80-3.94 (m, 5H), 4.06 (m, 1H), 4.20 (m,2H), 4.76 (m, 1H), 6.92 (d, 1H), 7.24 (m,1H), 7.58 (m, 1H)LRMS: m/z ES⁺ 317 [MNa]⁺ |
| 57⁷ | 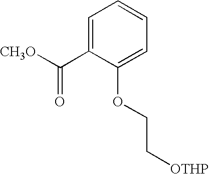 | 80 | ¹HNMR (CDCl₃, 400 MHz) δ: 1.50-1.90(m, 6H), 2.50 (t, 1H), 3.90 (m, 5H), 4.10(m, 1H), 4.25 (m, 2H), 4.80 (m, 1H), 7.00(m, 2H), 7.40 (dd, 1H), 7.80 (dd, 1H).LRMS: m/z ES⁺ 303 [MNa]⁺ |

1 = Methyl 3-chlorosalicylate (U.S. Pat. No. 4,895,860, pg 14) was the starting alcohol
2 = Methyl 4-chloro-2-hydroxybenzoate (EP 0234872, ex 2f) was the starting alcohol
3 = Methyl 5-chloro-2-hydroxybenzoate (EP 0234872 ex 2c) was used as the starting alcohol
4 = Methyl 2-hydroxy-3-methylbenzoate was used as the starting alcohol
5 = Methyl 2-hydroxy-4-methylbenzoate was used as the starting alcohol
6 = Methyl 2-hydroxy-5-methylbenzoate was used as the starting alcohol
7 = Methyl salicylate was used as the starting alcohol

### PREPARATIONS 58 to 64

A mixture of the appropriate ester from preparations 51 to 57 (1 eq) and lithium hydroxide (1M aqueous) (8-12 mlmmol⁻¹) in tetrahydrofuran (5-11 mlmmol⁻¹) was stirred at room temperature for 72 hours. The reaction mixture was concentrated under reduced pressure and the residue acidified using 10% aqueous citric acid solution. The aqueous solution was extracted with ethyl acetate, and the combined organic extracts were washed with brine, dried (MgSO₄) and evaporated under reduced pressure to afford the title compounds as clear oils.

| Prep | | |
|---|---|---|
| No | | Data |
| 58 | 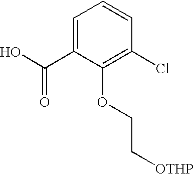 | ¹HNMR (CDCl₃, 400 MHz) δ: 1.46-1.94 (m, 6H),3.56 (m, 1H), 3.76-3.90 (m, 2H), 4.10 (m, 1H), 4.22(m, 1H), 4.52 (m, 1H), 4.74 (m, 1H), 7.23 (t, 1H),7.62 (m, 1H), 8.12 (m, 1H)LRMS: m/z ES⁺ 323 [MNa]⁺ |
| 59 | 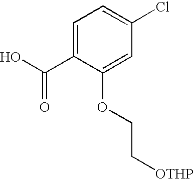 | ¹HNMR (CDCl₃, 400 MHz) δ: 1.40-1.90 (m, 6H),3.56 (m, 1H), 3.86 (m, 2H), 4.16 (m, 1H), 4.30-4.46(m, 2H), 4.74 (m, 1H), 7.06 (s, 1H), 7.14 (m, 1H),8.14 (d, 1H)LRMS: m/z ES⁺ 323 [MNa]⁺ |
| 60 | 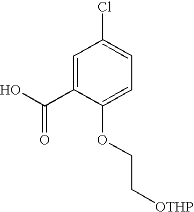 | ¹HNMR (CDCl₃, 400 MHz) δ: 1.48-1.90 (m, 6H),3.54 (m, 1H), 3.82 (m, 2H), 4.16 (m, 1H), 4.28-4.44(m, 2H), 4.78 (m, 1H), 7.00 (d, 1H), 7.50 (m, 1H),8.16 (d, 1H)LRMS: m/z ES⁺ 323 [MNa]⁺ |
| 61 | 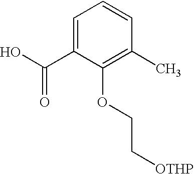 | ¹HNMR (CDCl₃, 400 MHz) δ: 1.46-1.96 (m, 6H),2.40 (s, 3H), 3.54 (m, 1H), 3.70-3.90 (m, 2H), 4.10(m, 1H), 4.18 (m, 2H), 4.74 (m, 1H), 7.20 (m, 1H),7.44 (d, 1H), 7.92-8.06 (m, 1H)LRMS: m/z ES⁺ 303 [MNa]⁺ |
| 62 | 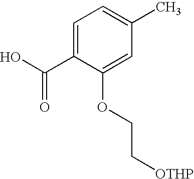 | ¹HNMR (CDCl₃, 400 MHz) δ: 1.44-1.94 (m, 6H),2.40 (s, 3H), 3.54 (m, 1H), 3.78-3.90 (m, 2H),4.08-4.20 (m, 1H), 4.32-4.46 (m, 2H), 4.74 (m,1H), 6.88 (s, 1H), 6.96 (m, 1H), 8.06 (d, 1H)LRMS: m/z ES⁺ 303 [MNa]⁺ |
| 63 | 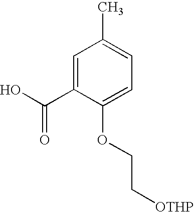 | ¹HNMR (CDCl₃, 400 MHz) δ: 1.44-1.94 (m, 6H),2.34 (s, 3H), 3.56 (m, 1H), 3.78-3.92 (m, 2H), 4.12(m, 1H), 4.30-4.44 (m, 2H), 4.72 (m, 1H), 6.96 (d,1H), 7.34 (m, 1H), 8.00 (s, 1H)LRMS: m/z ES⁺ 303 [MNa]⁺ |
| 64 | 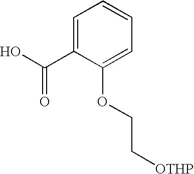 | ¹HNMR (CDCl₃, 400 MHz) δ: 1.50-1.92 (m, 6H),3.56 (m, 1H), 3.84 (m, 2H), 4.08-4.20 (m, 1H),4.34-4.48 (m, 2H), 4.74 (m, 1H), 7.06 (m, 1H), 7.16(m, 1H), 7.56 (m, 1H), 8.20 (m, 1H)LRMS: m/z ES⁺ 289 [MNa]⁺ |

### PREPARATION 65

### 2-Hydroxy-4-hydroxymethylbenzoic acid

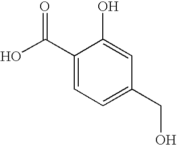

A mixture of 3-hydroxybenzylalcohol (10 g, 80 mmol) and potassium carbonate (33.35 g, 240 mmol) were stirred under carbon dioxide in a sealed vessel at 1500-2000 psi and 150° C. for 18 hours. The cooled residue was dissolved in water, acidified to pH 1 using concentrated hydrochloric acid and extracted with ethyl acetate. The combined organic extracts were washed with brine, dried (MgSO₄) and evaporated under reduced pressure. The product was recrystallised from cyclohexane/isopropyl acetate to afford the title compound, 740 mg.

¹HNMR (CD₃OD, 400 MHz) δ: 4.60 (s, 2H), 6.84 (m, 1H), 6.90 (s, 1H), 7.80 (d, 1H).

### PREPARATION 66

### 4-Ethyl-2-hydroxy-benzoic acid

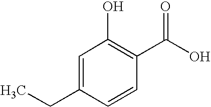

3-Ethyl phenol (10 g, 82 mmol) and potassium carbonate (34 g, 246 mmol) were heated in a sealed vessel at 150° C. under an atmosphere of carbon dioxide for 18 hours. The cooled mixture was dissolved in water, the solution acidified with concentrated hydrochloric acid, and the resulting precipitate filtered and dried to afford the title compound as a white solid, 11.45 g.

LRMS: m/z APCI⁻ 165 [M-H]⁻

### PREPARATION 67

### 2-Hydroxy-5-isopropyl-benzoic acid

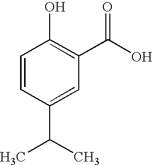

4-Isopropyl phenol (1.0 g, 7.3 mmol) and potassium carbonate (2.03 g, 14.7 mmol) were heated to 150° C. under an atmosphere of carbon dioxide. The cooled residue was suspended in ethyl acetate and acidified carefully with 2N hydrochloric acid. The layers were separated, the aqueous phase extracted with ethyl acetate and the combined extracts dried (MgSO₄) and evaporated under reduced pressure to give a tan-coloured solid, 1.23 g.

¹HNMR (CDCl₃, 400 MHz) δ: 1.20 (d, 6H), 2.90 (m, 1H), 6.80 (d, 1H), 6.90 (s, 1H), 7.80 (d, 1H), 10.40 (s, 1H).

### PREPARATION 68

### 2-Hydroxy-4-isopropyl-benzoic acid

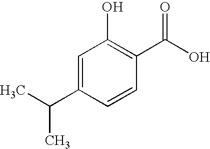

The title compound was obtained as a tan coloured solid from 3-isopropyl phenol, following the procedure described in preparation 67.

¹HNMR (CDCl₃, 400 MHz) δ: 1.20 (d, 6H), 2.90 (m, 1H), 7.00 (d, 1H), 7.40 (d, 1H), 7.70 (s, 1H), 10.20 (s, 1H).

### PREPARATION 69

### Benzyl 4-benzyloxy-2-hydroxybenzoate

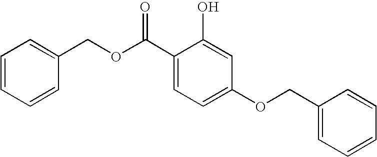

A mixture of benzyl bromide (111 g, 0.65 mol), potassium carbonate (90 g, 0.65 mol) and 2,4-dihydroxybenzoic acid (50 g, 0.32 mol) in N,N-dimethylformamide (250 ml) was stirred at room temperature for 18 hours.

The solid was filtered off, washing with N,N-dimethylformamide. Water (125 ml) was added to the filtrate, and the mixture extracted with ethyl acetate. The combined organic extracts were washed with 5% sodium hydroxide solution, dried (MgSO₄) and evaporated under reduced pressure. The crude product was recrystallised from 60/80 petroleum ether to give the title compound, 57.1 g.

¹HNMR (CDCl₃, 60 MHz) δ: 5.05 (s, 2H), 5.30 (s, 2H), 6.50 (m, 2H), 7.35 (m, 11H).

### PREPARATION 70

### 4-Benzyloxy-2-hydroxybenzoic acid

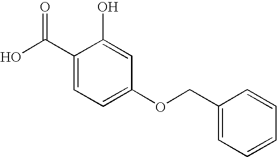

A solution of the compound from preparation 69 (9.0 g, 27 mmol) in 5% potassium hydroxide in ethanol was stirred under reflux for 6 hours. The mixture was concentrated under reduced pressure and the resulting solid dissolved in water and acidified using hydrochoric acid. The resulting solid was filtered off and recrystallised from toluene to afford the title compound, 3.1 g.

m.p. 179-180.5° C.

### PREPARATION 71

### 4-Fluoro-2-methoxy-benzonitrile

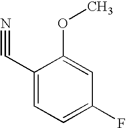

Potassium tert-butoxide (216 ml, 1M in tetrahydrofuran, 216 mmol) was added to ice-cooled methanol (8.7 ml, 216 mmol), and the solution stirred for 40 minutes. The resulting suspension was added dropwise to a solution of 2,4-difluorobenzonitrile (30 g, 216 mmol) in tetrahydrofuran at −78° C. Once addition was complete the reaction was allowed to warm to room temperature and stirred for 18 hours. The reaction was diluted with hexane (200 ml) and the mixture washed with water (200 ml), brine (2×200 ml), then dried (MgSO₄) and evaporated under reduced pressure. The residual solid was recrystallised from ethyl acetate:hexane to give the title compound, 9.8 g.

¹HNMR (CDCl₃, 300 MHz) δ: 3.90 (s, 3H), 6.70 (m, 2H), 7.55 (dd, 1H).

LRMS: m/z ES⁺ 152 [MH⁺]

### PREPARATION 72

### 4-Benzyloxy-2-methoxy-benzonitrile

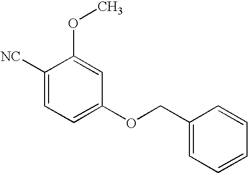

Potassium tert-butoxide (97 ml, 1M In tetrahydrofuran, 97 mmol) was added to an ice-cooled solution of benzyl alcohol (10.1 ml, 97 mmol) in tetrahydrofuran (50 ml). This solution was then added to a solution of the compound from preparation 71 (9.8 g, 65 mmol) in tetrahydrofuran (50 ml) and the reaction stirred at 40° C. for 5 hours. The mixture was diluted with ethyl acetate, and washed with water and brine. The solution was dried (MgSO₄) and evaporated under reduced pressure. The residue was recrystallised from ethyl acetate:hexane to give the title compound, 12.73 g.

¹HNMR (CDCl₃, 300 MHz) δ: 3.88 (s, 3H), 5.10 (s, 2H), 6.60 (m, 2H), 7.35-7.50 (m, 6H).

### PREPARATION 73

### 4-Benzyloxy-2-methoxy-benzoic acid

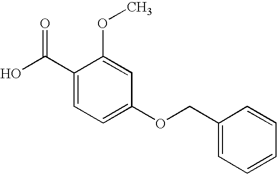

A solution of sodium hydroxide (6.7 g, 170 mmol) in water (50 ml) was added to a suspension of the compound from preparation 72 (10 g, 42 mmol) in ethanol (100 ml) and the reaction heated under reflux for 36 hours. Additional sodium hydroxide (2.0 g, 5 mmol) was added and the reaction heated for a further 24 hours. The cooled mixture was poured into ice/water (1 L), and acidified with concentrated hydrochloric acid. The resulting precipitate was filtered off and dried to give the title compound.

¹HNMR (CDCl₃, 300 MHz) δ: 3.98 (s, 3H), 5.10 (s, 2H), 6.80 (m, 2H), 7.40 (m, 5H), 7.52 (m, 1H).

### PREPARATION 74

### 4-Hydroxy-2-methoxy-benzoic acid

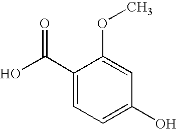

30% Palladium on charcoal (1.5 g) was added to a solution of the compound from preparation 73 (11.47 g, 44.4 mmol) in methanol (300 ml), and the mixture hydrogenated at 60 psi and room temperature for 24 hours. The mixture was filtered through silica and the filtrate evaporated under reduced pressure. The residue was recrystallised from ethyl acetate:hexane to give the title compound.

¹HNMR (CD₃OD, 300 MHz) δ: 3.80 (s, 3H), 6.35 (d, 1H), 6.45 (s, 1H), 7.55 (d, 1H).

### PREPARATION 75

### 5-Methoxy-benzo[1,2,5]thiadiazole-4-sulphonyl chloride

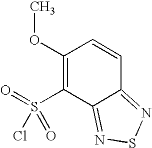

5-Methoxy-2,1,3-benzothiadiazole (500 mg, 3.01 mmol) was added to ice-cooled chlorosulphonic acid (11.0 ml, 15 mmol) and the reaction heated to 100° C. for 1 hour. The cooled mixture was poured into ice-water (15 ml), the resulting precipitate filtered off and dried to afford the title compound as a beige solid, 535 mg.

LRMS: m/z APCI⁺ 265, 267 [MH⁺]

### PREPARATION 76

### syn-[4-(2-Hydroxy-4-methyl-benzoylamino)-cyclohexyl]-carbamic acid tert-butyl ester

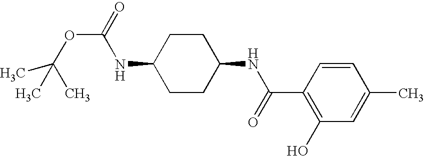

4-Methylsallylic acid (3.5 g, 23 mmol) was added to a mixture of the amine from preparation 5 (5.35 g, 25 mmol) 1-hydroxybenzotriazole hydrate (3.88 g, 28.8 mmol) 1-(3-dimethylaminopropyl-3-ethylcarbodiimide hydrochloride (6.23 g, 32.5 mmol) and N-diisopropylethylamine (4.84 g, 37.5 mmol) in dichloromethane (65 ml). The mixture was stirred at room temperature for 72 hours and was diluted with dichloromethane (100 ml). Water (150 ml) was added and the aqueous layer was acidified to pH 3 by addition of 2M hydrochloric acid. The phases were separated and the organic phase was washed with water (2×100 ml) and dried (MgSO₄). The organic solution was concentrated in-vacuo and the residue was triturated with hot ethyl acetate to give the title compound, 5.2 g.

LRMS: m/z ES⁺ 371 [MNa⁺]

### PREPARATION 77

### syn-N-(4-Amino-cyclohexyl)-2-hydroxy-4-methyl-benzamide hydrochloride

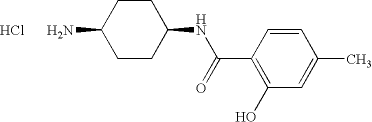

The compound from preparation 76 (5.1 g, 14.6 mmol) was suspended in dichloromethane (400 ml) and was cooled to 0° C. The mixture was purged under nitrogen and hydrogen chloride gas was bubbled into the mixture for 10 minutes to give a saturated solution. The reaction mixture was stirred at 4° C. for 3 hours and then concentrated in-vacuo. The residue was co-evaporated with dichloromethane (2×) and triturated with diethyl ether. The material obtained was isolated by filtration and was washed with diethyl ether to give the title compound as a white solid (4.21 g).

LRMS: m/z ES⁺ 249 [MH⁺]

### PREPARATION 78

### syn-2-Chloro-5-fluoro-N-[4-(2-hydroxy-4-methyl-benzoylamino)-cyclohexyl]-nicotinamide

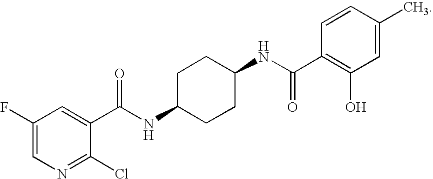

1-(3-Dimethylaminopropyl-3-ethylcarbodiimide hydrochloride (1.68 g, 5.85 mmol) was added to the compound from preparation 77 (2 g, 7.02 mmol) the acid from preparation 1 (1.03 g, 5.85 mmol), 1-hydroxybenzotriazole hydrate (0.95 g, 7.02 mmol) and N-diisopropylethylamine (4.6 ml, 26.3 mmol) in dichloromethane (50 ml) and the mixture was stirred at room temperature under a nitrogen atmosphere for 16 hours. Additional 1-(3-dimethylaminopropyl-3-ethylcarbodiimide hydrochloride (0.56 g, 2.9 mmol) was added and the mixture was stirred for a further 2 hours. The reaction mixture was partitioned between 1N hydrochloric acid and dichloromethane. The phases were separated and the aqueous layer was extracted with dichloromethane (2×). The combined organic solutions were dried (MgSO₄) and concentrated in-vacuo. The material obtained was recrystallised from isopropyl acetate to give the title compound as a white solid (1.3 g).

LRMS: m/z ES⁺ 406 [MH⁺]

### PREPARATION 79

### Syn-N-[4-(2-Benzyloxy-5-trifluoromethyl-benzoylamino)-cyclohexyl]-5-fluoro-2-(tetrahydro-thiopyran-4-yloxy)-nicotinamide

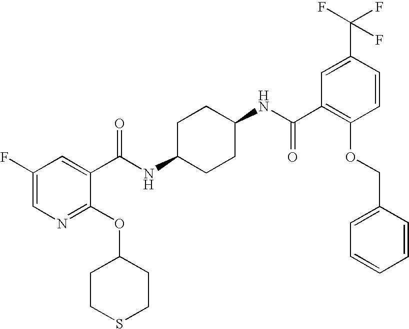

1-(3-Dimethylaminopropyl-3-ethylcarbodiimide hydrochloride (374 mg, 1.95 mmol) was added to a mixture of the amine from preparation 15b (530 mg, 1.5 mmol), 2-benzyloxy-5-trifluoromethylbenzoic acid (U.S. Pat. No. 3,953,595, pg 9), (400 mg, 1.35 mmol), 1-hydroxybenzotriazole hydrate (264 mg, 1.96 mmol) and N-ethyldiisopropylamine (0.78 ml, 4.5 mmol) in N,N-dimethylformamide (30 ml) and the reaction stirred at room temperature for 18 hours: The mixture was diluted with ethyl acetate (20 ml), and washed with 1N citric acid (20 ml), saturated sodium bicarbonate solution (20 ml), then dried (MgSO₄) and evaporated under reduced pressure. The resulting solid was triturated with ether to give the title compound as a white solid, 728 mg.

¹HNMR (CD₃OD, 400 MHz) δ: 1.48 (m, 2H), 1.60 (m, 2H), 1.75 (m, 4H), 1.89 (m, 2H), 2.30 (m, 2H), 2.69 (m, 4H), 3.97 (m, 2H), 5.30 (m, 3H), 7.18 (m, 1H), 7.31 (m, 2H), 7.45 (d, 1H), 7.53 (d, 2H), 7.81 (d, 1H), 8.09 (m, 1H), 8.20 (m, 2H).

LRMS: m/z ES⁺ 654 [MNa]⁺

### PREPARATION 80

### Syn-5-Fluoro-N-(4-{2-[2-(tetrahydro-pyran-2-yloxy)-ethoxy]-benzoylamino}-cyclohexyl)-2-(tetrahydro-thiopyran-4-yloxy-nicotinamide

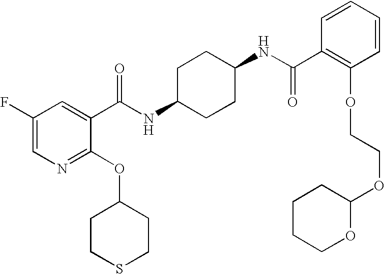

A mixture of the amine from preparation 15a (200 mg, 0.51 mmol), the acid from preparation 64 (150 mg, 0.56 mmol), 1-(3-dimethylaminopropyl-3-ethylcarbodiimide hydrochloride (150 mg, 0.78 mol), 1-hydroxybenzotriazole hydrate (80 mg, 0.59 mmol) and N-ethyldiisopropylamine (225 μl, 1.29 mmol) in N,N-dimethylformamide (2 ml) was stirred at room temperature for 18 hours. The mixture was partitioned between ethyl acetate and 10% citric acid solution and the layers separated. The organic phase was washed with further 10% citric acid, saturated aqueous sodium bicarbonate solution, brine, then dried (MgSO₄) and evaporated under reduced pressure to give the title compound as a gum, 260 mg.

¹HNMR (CD₃OD, 400 MHz) δ: 1.27-2.02 (m, 15H), 2.40 (m, 2H), 2.65-2.79 (m, 4H), 3.38 (m, 1H), 3.72 (m, 2H), 3.88 (m, 1H), 4.02-4.16 (m, 4H), 4.37 (t, 3H), 4.58 (m, 1H), 5.31 (m, 1H), 7.07 (t, 1H), 7.16 (d, 1H), 7.47 (t, 1H), 7.95 (d, 1H), 8.06 (m, 1H), 8.17 (m, 1H), 8.43 (m, 1H).

LRMS: m/z APCI⁺ 518 [MH-THP]⁺

### PREPARATION 81

### Syn-1-[2-(Tetrahydro-pyran-2-yloxy)-ethyl]-1H-indazole-3-carboxylic acid (4-{[5-fluoro-2-(tetrahydro-thiopyran-4-yloxy)-pyridine-3-carbonyl]-amino}-cyclohexyl)-amide

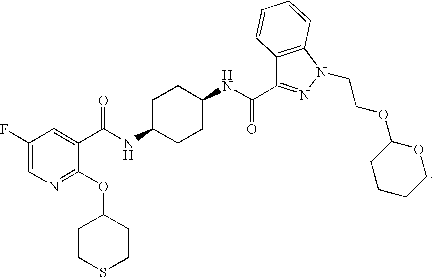

The title compound was obtained as a white solid, from the amine from preparation 15a and the acid from preparation 32, following a similar procedure to that described in preparation 80, except 1-methyl-2-pyrrolidinone was used as the reaction solvent.

¹HNMR (CD₃OD, 400 MHz) δ: 1.20-1.53 (m, 6H), 1.78 (m, 8H), 1.94 (m, 2H), 2.27 (m, 2H), 2.65 (m, 22H), 2.80 (m, 2H), 3.22 (m, 1H), 3.30 (m, 1H), 3.80 (m, 1H), 3.97 (m, 3H), 4.47 (m, 1H), 4.65 (m, 2H), 5.20 (m, 1H), 7.23 (m, 1H), 7.43 (m, 1H), 7.65 (d, 1H), 7.78 (d, 1H), 7.97 (m, 1H), 8.10 (m, 2H), 8.29 (s, 1H).

LRMS: m/z ES⁺ 648 [MH]⁺

### PREPARATION 82

### Syn-5-Fluoro-N-[4-({5-methyl-1-[2-(tetrahydro-pyran-2-yloxy)-ethyl]-1H-pyrazole-3-carbonyl}-amino)-cyclohexyl]-2-(tetrahydro-thiopyran-4-yloxy)-nicotinamide

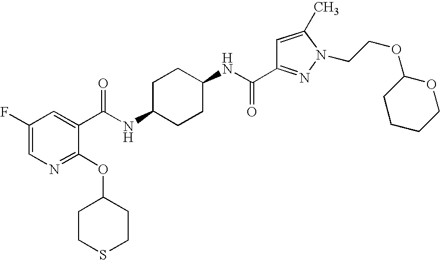

A mixture of the amine from preparation 15a (190 mg, 0.48 mmol), the acid from preparation 39 (125 mg, 0.49 mmol), 1-(3-dimethylaminopropyl-3-ethylcarbodiimide hydrochloride (140 mg, 0.73 mol), 1-hydroxybenzotriazole hydrate (70 mg, 0.52 mmol) and N-ethyldiisopropylamine (260 μl, 1.44 mmol) In N,N-dimethylformamide (3 ml) was stirred at room temperature for 18 hours. The mixture was partitioned between ethyl acetate (50 ml) and 10% citric acid solution (50 ml) and the layers separated. The organic phase was washed with further 10% citric acid, saturated aqueous sodium bicarbonate solution, brine, then dried (MgSO₄) and evaporated under reduced pressure to give the title compound as a gum, 260 mg.

¹HNMR (CDCl₃, 400 MHz) δ: 1.42-2.04 (m, 15H), 2.32-2.48 (m, 6H), 2.81 (m, 4H), 3.41 (m, 1H), 3.55 (m, 1H), 3.77 (m, 1H), 4.00-4.29 (m, 5H), 4.50 (m, 1H), 5.31 (m, 1H), 6.57 (s, 1H), 7.07 (m, 1H), 8.01-8.13 (m, 2H), 8.26 (m, 1H)

LRMS: m/z APCI⁺ 590 [MH]⁺

### PREPARATION 83

### Syn-N-[4-(4-Benzyloxy-2-ethoxy-benzoylamino)-cyclohexyl]-5-fluoro-2-(tetrahydro-thiopyran-4-yloxy)-nicotinamide

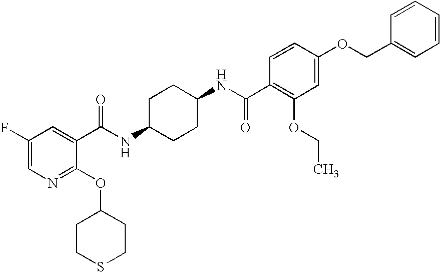

Potassium carbonate (86 mg, 0.62 mmol) and potassium iodide (5 mg, 0.03 mmol) were added to a solution of the phenol from example 79 (180 mg, 0.31 mmol) in acetonitrile (5 ml) and N,N-dimethylformamide (1 ml). Ethyl bromide (30 μl, 0.4 mmol) was added and the mixture stirred at 35° C. for 18 hours. The mixture was evaporated under reduced pressure and the residue partitioned between ethyl acetate and 1N hydrochloric acid, and the layers separated. The organic phase was washed with water, sodium carbonate solution and brine, then dried (MgSO₄) and evaporated under reduced pressure. The product was purified by column chromatography on silica gel using an elution gradient of ethyl acetate:pentane (20:80 to 70:30) to afford the title compound as an oil, 174 mg.

¹HNMR (CDCl₃, 400 MHz) δ: 1.52 (t, 3H), 1.60-2.08 (m, 10H), 2.42 (m, 2H), 2.74 (m, 4H), 4.08-4.22 (m, 4H), 5.10 (s, 2H), 5.26 (m, 1H), 6.56 (d, 1H), 6.68 (m, 1H), 7.32-7.46 (m, 5H), 8.04 (m, 3H), 8.20 (d, 1H), 8.26 (m, 1H)

LRMS: m/z ES⁺ 608 [MH]⁺

### PREPARATION 84

### Syn-N-[4-(4-Benzyloxy-2-cyclopropylmethoxy-benzoylamino)cyclohexyl]-5-fluoro-2-(tetrahydro-thiopyran-4-yloxy)-nicotinamide

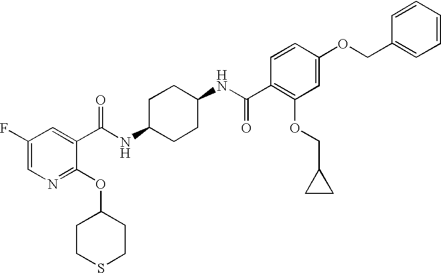

The title compound was obtained in 87% yield from the phenol from example 79 and (bromomethyl)cyclopropane, following the procedure described in preparation 83, except the reaction was performed at 90° C.

¹HNMR (CDCl₃, 400 MHz) δ: 0.38 (m, 2H), 0.64 (m, 2H), 1.20-1.38 (m, 1H), 1.64-2.04 (m, 10H), 2.40 (m, 2H), 2.74 (m, 4H), 3.92 (d, 2H), 4.04-4.22 (m, 2H), 5.10 (s, 2H), 5.24 (m, 1H), 6.50 (d, 1H), 6.68 (m, 1H), 7.30-7.46 (m, 5H), 8.02 (m, 2H), 8.16-8.28 (m, 3H)

LRMS: m/z ES⁺ 656 [MNa]⁺

### PREPARATION 85

### Syn-N-[4-(4-Benzyloxy-2-cyclopentoxy-benzoylamino)-cyclohexyl]-5-fluoro-2-(tetrahydro-thiopyran-4-yloxy)-nicotinamide

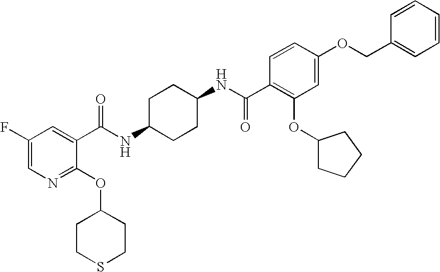

The title compound was obtained in 87% yield from the phenol from example 79 and cyclopentylbromide, following the procedure described in preparation 83, except the reaction was performed at 90° C.
¹HNMR (CDCl₃, 400 MHz) δ: 1.38-2.06 (m, 18H), 2.24 (m, 2H), 2.68-2.72 (m, 4H), 4.12 (m, 2H), 4.90 (m, 1H), 5.10 (s, 1H), 5.24 (m, 1H), 6.46 (d, 1H), 6.66 (m, 1H), 7.30-7.48 (m, 5H), 7.94 (d, 1H), 8.04 (m, 2H), 8.16 (d, 1H), 8.28 (m, 1H)

LRMS: m/z ES⁺ 670 [MNa]⁺

### PREPARATION 86

### Syn-5-Fluoro-N-(4-{5-methyl-2-[2-(tetrahydro-pyran-2-yloxy)-ethoxy]-benzoy lamino}-cyclohexyl)-2-(tetrahydro-thiopyran-4-yloxy)-nicotinamide

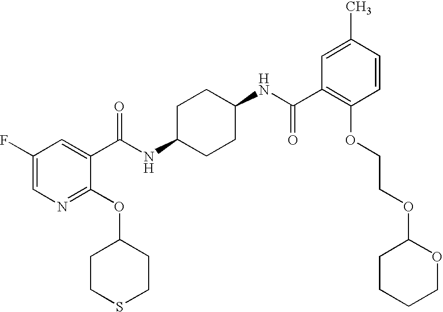

A mixture of the phenol from example 22 (1.29 g, 2.65 mmol), potassium carbonate (690 mg, 5 mmol), and 2-(2-bromoethoxy)tetrahydro-2H-pyran (840 mg, 4 mmol) in 1-methyl-2-pyrrolidinone (10 ml), was heated at 60° C. for 4 hours, followed by a further 18 hours at room temperature. The mixture was diluted with ethyl acetate and washed with water (×3), then brine, dried (MgSO₄) and evaporated under reduced pressure. The residue was purified by column chromatography on silica gel using ethyl acetate as the eluant to afford the title compound as a white foam, 1.20 g.

¹HNMR (CDCl₃, 400 MHz) δ: 1.37 (m, 2H), 1.46 (m, 2H), 1.61 (m, 4H), 1.76 (m, 4H), 1.92 (m, 4H), 2.33 (s, 3H), 2.43 (m, 2H), 2.72 (m, 4H), 3.39 (m, 1H), 3.72 (m, 1H), 3.86 (m, 1H), 4.13 (m, 3H), 4.30 (t, 2H), 4.54 (m, 1H), 5.24 (m, 1H), 6.87 (d, 1H), 7.21 (d, 1H), 8.04 (m, 3H), 8.13 (d, 1H), 8.26 (dd, 1H).

LRMS: m/z APCI⁻ 614 [M-H⁻]

### PREPARATION 87

### 1H-Indazole-7-carboxylic acid

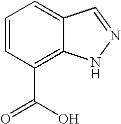

A solution of sodium nitrite (1.9 g, 27.6 mmol) in water (5 ml) was added dropwise to an ice-cooled solution of methyl 2-amino-3-methyl benzoate (U.S. Pat. No. 4,657,893 preparation II) (4.14 g, 25 mmol) in acetic acid (50 ml). This solution was then added dropwise to a solution of tert-butyl mercaptan (2.26 g, 25 mmol) in ethanol (70 ml) and stirred at room temperature. The pH of the mixture was adjusted to 5.5 using saturated sodium carbonate solution and the mixture poured into brine. This mixture was extracted with ethyl acetate, the combined organic extracts dried (Na₂SO₄), concentrated under reduced pressure and the residue azeotroped with dichloromethane and heptane. The residue was dissolved in dimethyl sulphoxide (40 ml) and added dropwise to a suspension of potassium tert-butoxide (14.05 g, 126 mmol) in dimethyl sulphoxide (150 ml), and the reaction stirred at room temperature for 2 hours. The reaction was poured carefully into 1N hydrochloric acid, and extracted with ethyl acetate. The combined organic extracts were washed with 1N hydrochloric acid, dried (Na₂SO₄) and evaporated under reduced pressure. The product was slurried with Isopropanol, sufficient dichloromethane added for complete dissolution, and the solution allowed to evaporate. The resulting solid was filtered off, washed with isopropanol to afford the title compound as an off-white solid.

Microanalysis found: C, 59.26; H, 3.73; N, 17.28. C₈H₆N₂O₂ requires C, 59.31; H, 3.51; N. 17.42%.

Mpt. 230-233° C.

### PREPARATION 88

### Syn-N-[4-(4-Benzyloxy-2-hydroxy-benzoylamino)-cyclohexyl]-5-fluoro-2-(tetrahydro-thiopyran-4-yloxy)-nicotinamide

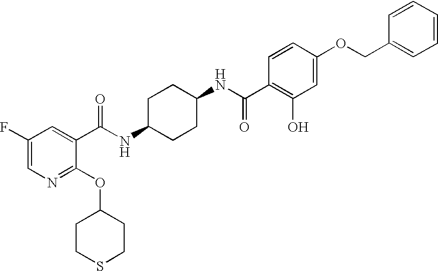

The title compound was obtained as an oil in 32% yield from the amine from preparation 15a and the acid from preparation 70, following a similar procedure to that described in examples 6 to 14, except N,N-dimethylformamide was used as the reaction solvent.

¹HNMR (CDCl₃, 400 MHz) δ: 1.60-2.10 (m, 10H), 2.40 (m, 2H), 2.70-2.90 (m, 4H), 4.14 (m, 1H), 4.28 (m, 1H), 5.08 (s, 2H), 5.48 (m, 1H), 6.16 (m, 1H), 6.48 (m, 1H), 6.56 (d, 1H), 7.30-7.46 (m, 6H), 8.04-8.14 (m, 2H), 8.28 (m, 1H)

LRMS: m/z ES⁺ 602 [MNa]⁺

Microanalysis found; C, 64.09; H, 5.96; N, 7.08, C₃₁H₃₄FN₃O₅S; requires C, 64.23; H, 5.91; N, 7.25%.

### PREPARATION 89

### Syn-5-Fluoro-N-[4-(2-hydroxy-benzoylamino)-cyclohexyl]-2-(tetrahydro-thiopyran-4-yloxy)-nicotinamide

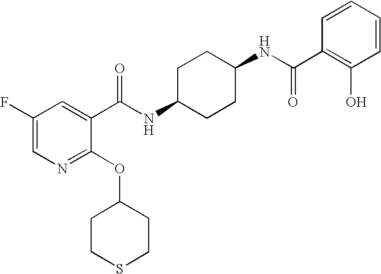

A solution of 2-hydroxybenzoic acid (32 mg, 0.21 mmol) In N,N-dimethylformamide (0.5 ml) was added to a mixture of 1-(3-dimethylaminopropyl)-3-ethylcarbodiimide hydrochloride (44 mg, 0.23 mmol), the amine from preparation 15a (82 mg, 0.21 mmol), 1-hydroxybenzotriazole hydrate (31 mg, 0.23 mmol) and N-methylmorpholine (48 μl, 0.44 mmol) in N,N-dimethylformamide (4 ml), and the reaction stirred at room temperature for 18 hours. The mixture was evaporated under reduced pressure and the residue suspended in tetrahydrofuran (1 ml) and 1N sodium hydroxide solution (1 ml), and stirred at room temperature for 72 hours. The mixture was concentrated under reduced pressure, the aqueous solution acidified by the addition of 2N hydrochloric acid (1 ml), and extracted with dichloromethane (5 ml, 2 ml). The combined organic extracts were washed with water (2 ml), dried (MgSO₄) and evaporated under reduced pressure. The residue was crystallised from methanol to afford the title compound in 86% yield.

¹HNMR (CDCl₃, 400 MHz) δ: 1.72(m, 2H), 1.81 (m, 2H), 1.97 (m, 6H), 2.39 (m, 2H), 2.80 (m, 4H), 4.15 (m, 1H), 4.26 (m, 1H), 5.47 (m, 1H), 6.37 (m, 1H), 6.87 (t, 1H), 6.99 (d, 1H), 7.42 (m, 2H), 8.06 (d, 1H), 8.11 (m, 1H), 8.28 (m, 1H), 12.22 (brs, 1H)

LRMS: m/z ES⁺ 496 [MNa]⁺

Microanalysis found; C, 60.60; H, 5.96; N, 8.71, C₂₄H₂₈FN₃O₄S; requires C, 60.87; H, 5.96; N, 8.87%.

### In Vitro Activity of the Nicotinamide Derivatives (I)

The PDE4 inhibitory activity of the nicotinamide derivatives of the formula (I) is determined by the ability of compounds to inhibit the hydrolysis of CAMP to AMP by PDE4 (Thompson J W, Teraski W L, Epstein P M, Strada S J., “Assay of nucleotidephosphodiesterase and resolution of multiple molecular forms of the Isoenzyme”, Advances in cyclic nucleotides research, edited by Brooker G, Greengard P, Robinson G A. Raven Press, New York 1979, 10, p. 69-92). Tritium labelled cAMP is incubated with PDE4. Following incubation, the radiolabelled AMP produced is able to bind yttrium silicate SPA beads. These SPA beads subsequently produce light that can be quantified by scintillation counting. The addition of a PDE4 inhibitor prevents the formation of AMP from cAMP and counts are diminished. The IC₅₀ of a PDE4 inhibitor can be defined as the concentration of a compound that leads to a 50% reduction in counts compared to the PDE4 only (no inhibitor) control wells.

The anti-inflammatory properties of the nicotinamide derivatives of the formula (I) are demonstrated by their ability to inhibit TNFα release from human peripheral blood mononuclear cells (see also Yoshimura T. Kurita C, Nagao T. Usami E, Nakao T, Watanabe S, Kobayashi J, Yamazaki F, Tanaka H, Nagai H., “Effects of CAMP-phosphodiesterase isozyme inhibitor on cytokine production by lipopolysaccharide-stimulated human peripheral blood mononuclear cells”, Gen. Pharmacol., 1997, 29(4), p. 63). Venous blood is collected from healthy volunteers and the mononuclear cells purified by centrifugation through Histopaque (Ficoll) cushions. TNFα production from these cells is stimulated by addition of lipopolysaccharide. After 18 hours incubation in the presence of LPS, the cell supernatant is removed and the concentration of TNFα in the supernatant determined by ELISA. Addition of PDE4 inhibitors reduces the amount of TNFα produced. An IC₅₀ is determined which is equal to the concentration of compound that gives 50% inhibition of TNFα production as compared to the LPS stimulated control wells.

All the examples were tested in the assay described above and found to have an IC₅₀ (TNFα screen) of less than 300 nM. And for most of the tested compounds, they were found to have an IC₅₀ (TNFα screen) of even less than 100 nM.

Data are presented below for the Examples in which the TNFα and PDE4 inhibition are presented as IC₅₀ values in nM.

| | IC₅₀ | IC₅₀ |
|---|---|---|
| | (TNFα screen) | (PDE4 inhibition) |
| Example No. | in nM | in nM |
| 1 | 0.7 | |
| 2 | 1.5 | |
| 3 | 0.02 | |
| 4 | 0.04 | 1.5 |
| 5 | 0.02 | 1 |
| 6 | 13 | |
| 7 | 0.09 | |
| 8 | 0.04 | |
| 9 | 0.1 | 2.8 |
| 10 | 0.04 | 5 |
| 11 | 0.1 | |
| 12 | 1.5 | |
| 13 | 4 | |
| 14 | 0.1 | |
| 15 | 0.1 | |
| 16 | 0.8 | |
| 17 | 10 | |
| 18 | 10 | |
| 19 | 2 | |
| 20 | 0.1 | |
| 21 | 1 | |
| 22 | 0.2 | |
| 23 | 0.2 | |
| 24 | 0.1 | |
| 25 | 0.03 | |
| 26 | 1 | |
| 27 | 5 | |
| 28 | 0.2 | |
| 29 | 0.4 | |

1. A compound of formula (I), 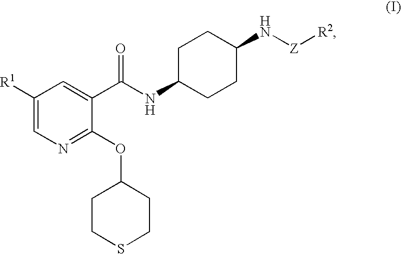
or a pharmaceutically acceptable salt thereof,
wherein:
R¹ is H, halo or (C₁-C₄)alkyl;
Z is CO or SO₂;
R² is phenyl, benzyl, naphthyl, heteroaryl or (C₃-C₈)cycloalkyl;
said phenyl, benzyl, naphthyl, heteroraryl and (C₃-C₈)cycloalkyl are each substituted with one (C₁-C₆)alkoxy, ((C₃-C₈)cycloalkyl)-(C₁-C₆)alkoxy, hydroxy(C₂-C₆)alkoxy, ((C₃-C₈)cycloalkyl)oxy or (C₁-C₆)alkoxyphenyl optionally substituted with one to three OH or halo;
said phenyl, benzyl, naphthyl, heteroraryl and (C₃-C₈)cycloalkyl are each additionally optionally substituted independently with; one or two halo: CN: CONR³R⁴: (C₁-C₆)alkyl optionally substituted with one to three halo; OH; hydroxy(C₁-C₆)alkyl; ((C₃-C₈)cycloalkyl)-(C₁-C₆)alkyl: (C₃-C₈)cycloalkyl; or NR³R⁴; and
R³ and R⁴ are each independently H, (C₁-C₄)alkyl or SO₂(C₁-C₄)alkyl.

2. A compound, of claim 1, or a Pharmaceutically acceptable salt thereof, wherein R¹ is H, halo, CH₃ or C₂H₅.

3. A compound, of claim 1, or a pharmaceutically acceptable salt thereof, wherein R² is phenyl, imidazolyl, pyrazinyl, indazolyl, purinyl, quinolyl, quinazolinyl, benzofuranyl, dihydrobenzofuranyl, benzothiadiazolyl, benzoxadiazolyl, pyrazolyl, imidazopyridyl, benzimidazolyl, pyrazolopyridyl, pyrazolopyrimidinyl, benzyl or cyclopropyl;
said phenyl, imidazolyl, pyrazinyl, indazolyl, purinyl, quinolyl, quinazolinyl, benzofuranyl, dihydrobenzofuranyl, benzothiadiazolyl, benzoxadiazolyl, pyrazolyl, imidazopyridyl, benzimidazolyl, pyrazolopyridyl, pyrazolopyrimidinyl, benzyl and cyclopropyl are each substituted with one (C₁-C₆)alkoxy, ((C₃-C₈)cycloalkyl)-(C₁-C₆)alkoxy, hydroxy(C₂-C₆)alkoxy, ((C₃-C₈)cycloalkyl)oxy, or (C₁-C₆)alkoxyphenyl optionally substituted with one to three OH or halo:
said phenyl, imidazolyl, pyrazinyl, indazolyl, purinyl, quinolyl, quinazolinyl, benzofuranyl, dihydrobenzofuranyl, benzothiadiazolyl, benzoxadiazolyl, pyrazolyl, imidazopyridyl, benzimidazolyl, pyrazolopyridyl, pyrazolopyrimidinyl, benzyl and cyclopropyl are each additionally optionally substituted independently with one or two halo; CN; CONR³R⁴; (C₁-C₆)alkyl optionally substituted with one to three halo; OH; hydroxy(C₁-C₆)alkyl; ((C₃-C₈)cycloalkyl)-(C₁-C₆)alkyl; (C₃-C₈)cycloalkyl; or NR³R⁴.

4. A compound of claim 1, or a pharmaceutically acceptable salt or solvate thereof, wherein R¹ is H, F, Cl or CH₃.

5. A compound, of claim 1, or a pharmaceutically acceptable salt or solvate thereof,
wherein R² is phenyl, imidazolyl, indazolyl, quinolyl, quinazolinyl, dihydrobenzofuranyl, benzothiadiazolyl, benzoxadiazolyl, pyrazolyl, imidazopyridyl, benzimidazolyl, pyrazolopyridyl, benzyl or cyclopropyl;
said Phenyl, imidazolyl, indazolyl, quinolyl, quinazolinyl, dihydrobenzofuranyl, benzothiadiazolyl, benzoxadiazolyl, pyrazolyl, imidazopyridyl, benzimidazolyl, pyrazolopyridyl, benzyl and cyclopropyl are each substituted with one OCH₃, OC₂H₄OH, O(CH₂)₃OH, OC₂H₅, cyclopropylmethoxy or cyclopentyloxy; and
said phenyl, imidazolyl, indazolyl, quinolyl, quinazolinyl, dihydrobenzofuranyl, benzothiadiazolyl, benzoxadiazolyl, pyrazolyl, imidazopyridyl, benzimidazolyl, pyrazolopyridyl, benzyl and cyclopropyl are each additionally optionally substituted independently with one or two CH₃, N(CH₃)SO₂CH₃, NHSO₂CH₂CH₃, NHSO₂CH(CH₃)₂, OH, CH₂OH, Cl, F, C₂H₅, CH(CH₃)₂, C₂H₄OH, or CF₃.

6. A compound, of claim 1, or a pharmaceutically acceptable salt or solvate thereof, wherein R¹ is F.

7. A compound, of claim 1, or a Pharmaceutically acceptable salt or solvate thereof, wherein Z is CO.

8. A compound, of claim 1, or a Pharmaceutically acceptable salt or solvate thereof, wherein R² is

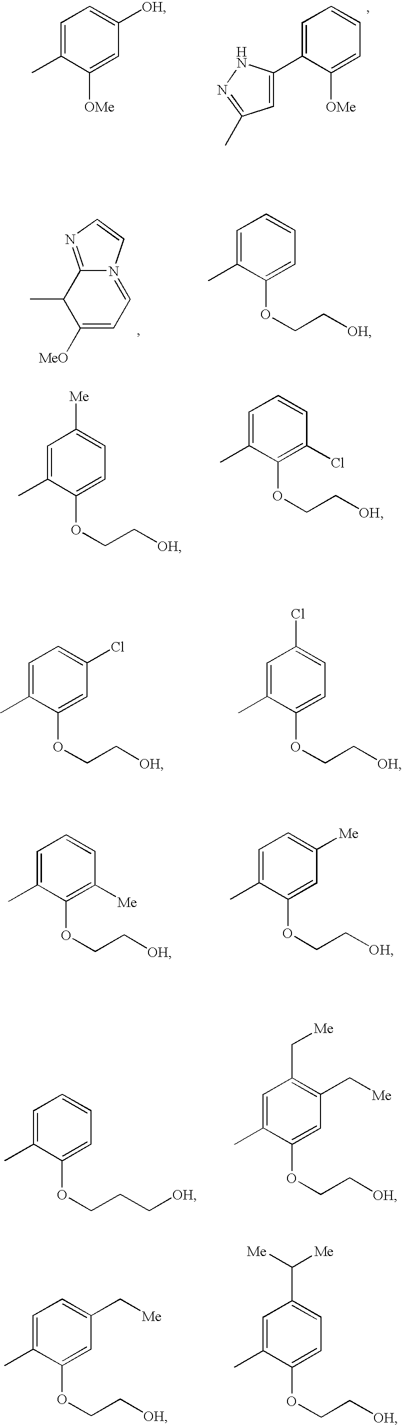

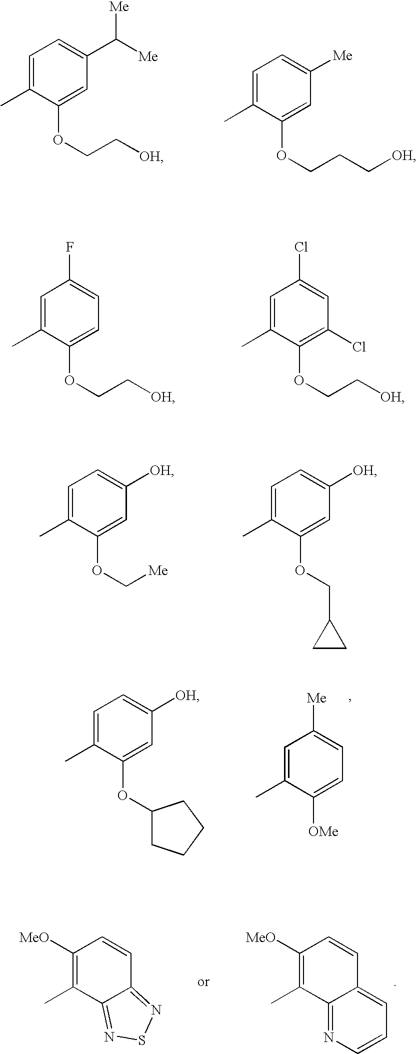

9. A compound, of claim, or a pharmaceutically acceptable salt or solvate thereof, wherein R² is

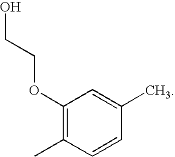

10.
Syn-5-Fluoro-N-[4-(4-hydroxy-2-methoxy-benzoylamino)-cyclohexyl]-2-(tetrahydro-thiopyran-4-yloxy)-nicotinamide:
Syn-5-Fluoro-N-(4-{[5-(2-methoxy-phenyl)-1H-pyrazole-3-carbonyl]-amino}-cyclohexyl)-2-(tetrahydro-thiopyran-4-yloxy)-nicotinamide;
Syn-7-Methoxy-imidazo[1,2-a]pyridine-8-carboxylic acid (4-{[5-fluoro-2-(tetrahydro-thiopyran-4-yloxy)-pyridine-3-carbonyl]-amino}-cyclohexyl)-amide;
Syn-5-Fluoro-N-{4-[2-(2-hydroxy-ethoxy)-benzoylamino]-cyclohexyl}-2-(tetrahydro-thiopyran-4-yloxy)-nicotinamide;
Syn-5-Fluoro-N-{5-[2-(2-hydroxy-ethoxy)-4-methyl-benzoylamino]-cyclohexyl}-2-(tetrahydro-thiopyran-4-yloxy)-nicotinamide;
Syn-N-{4-[3-Chloro-2-(2-hydroxy-ethoxy)-benzoylamino]-cyclohexyl}-5-fluoro-2-(tetrahydro-thiopyran-4-yloxy)-nicotinamide;
Syn-N-{4-[4-Chloro-2-(2-hydroxy-ethoxy)-benzoylamino]-cyclohexyl}-5-fluoro-2-(tetrahydro-thiopyran-4-yloxy)-nicotinamide;
Syn-N-{4-[5-Chloro-2-(2-hydroxy-ethoxy)-benzoylamino]-cyclohexyl}-5-fluoro-2-(tetrahydro-thiopyran-4-yloxy)-nicotinamide;
Syn-N-{4-[2-(2-hydroxy-ethoxy)-5-methyl-benzoylamino]-cyclohexyl}-2-(tetrahydro-thiopyran-4-yloxy)-nicotinamide;
Syn-N-{4-[2-(2-hydroxy-ethoxy)-3-methyl-benzoylamino]-cyclohexyl}-5-fluoro-2-(tetrahydro-thiopyran-4-yloxy)-nicotinamide;
Syn-N-{4-[2-(2-hydroxy-ethoxy)-4-methyl-benzoylamino]-cyclohexyl}-5-fluoro-2-(tetrahydro-thiopyran-4-yloxy)-nicotinamide;
Syn-N-{4-[2-(2-hydroxy-ethoxy)-4-methyl-benzoylamino]-cyclohexyl}-2-tetrahydro-thiopyran-4-yloxy)-nicotinamide;
Syn-N-{4-[5-Ethyl-2-(2-hydroxy-ethoxy)-benzoylamino]-cyclohexyl}-5-fluoro-2-(tetrahydro-thiopyran-4-yloxy)-nicotinamide;
Syn-N-{4-[4-Ethyl-2-(2-hydroxy-ethoxy)-benzoylamino]-cyclohexyl}-5-fluoro-2-(tetrahydro-thiopyran-4-yloxy)-nicotinamide;
Syn-N-{4-[2-(2-hydroxy-ethoxy)-4-isopropyl-benzoylamino]-cyclohexyl}-5-fluoro-2-(tetrahydro-thiopyran-4-yloxy)-nicotinamide;
Syn-N-{4-[2-(2-(2-hydroxy-ethoxy)-5-isopropyl-benzoylamino]-cyclohexyl}-5-fluoro-2-(tetrahydro-thiopyran-4-yloxy)-nicotinamide;
Syn-5-Fluoro-N-{4-[2-(3-hydroxy-propoxy)-4-methyl-benzoylamino]-cyclohexyl}-2-(tetrahydro-thiopyran-4-yloxy)-nicotinamide;
Syn-5-Fluoro-N-{4-[5-fluoro-2-(2-hydroxy-ethoxy)-benzoylamino]-cyclohexyl}-2-(tetrahydro-thiopyran-4-yloxy)-nicotinamide; or
Syn-N-{4-[3,5-Dichloro-2-(2-hydroxy-ethoxy)-benzoylamino]-cyclohexyl}-5-fluoro-2-(tetrahydro-thiopyran-4-yloxy)-nicotinamide;
Syn-N-{4-[2-(2-Hydroxy-ethoxy)-benzoylamino]-cyclohexyl}-5-methyl-2-(tetrahydro-thiopyran-4-yloxy)-nicotinamide;
Syn-5-Fluoro-N-{4-[2-(3-hydroxy-propoxy)-benzoylamino]-cyclohexyl}-2-(tetrahydro-thiopyran-4-yloxy)-nicotinamide;
Syn-N-[4-(2-Ethoxy-4-benzoylamino-cyclohexyl]-5-fluoro-2-(tetrahydro-thiopyran-4-yloxy)-nicotinamide;
Syn-N-[4-(2-Ethoxy-4-hydroxy-benzoylamino)-cyclohexyl]-5-fluoro-2-(tetrahydro-thiopyran-4-yloxy)-nicotinamide;
Syn-N-[4-(2-Cyclopentoxy-4-hydroxy-benzoylamino)-cyclohexyl]5-fluoro-2-(tetrahydro-thiopyran-4-yloxy)-nicotinamide;
Syn-N-[4-(2-methoxy-5-methyl-benzenesulfonylamino)-cyclohexyl]-2-(tetrahydro-thiopyran-4-yloxy)-nicotinamide;
Syn-5-Fluoro-N-[4-(7-methoxy-quinoline-8-sulfonylamino)-cyclohexyl]-2-(tetrahydro-thiopyran-4-yloxy)-nicotinamide; or
Syn-N-[4-(7-methoxy-quinoline-8-sulfonylamino)-cyclohexyl]-2-(tetrahydro-thiopyran-4-yloxy)-nicotinamide, or
a Pharmaceutically acceptable salt or solvate thereof.

11. Syn-5-Fluoro-N-{5-[2-(2-hydroxy-ethoxy)-4-methyl-benzoylamino]-cyclohexyl}-2-(tetrahydro-thiopyran-4-yloxy)-nicotinamide of formula: 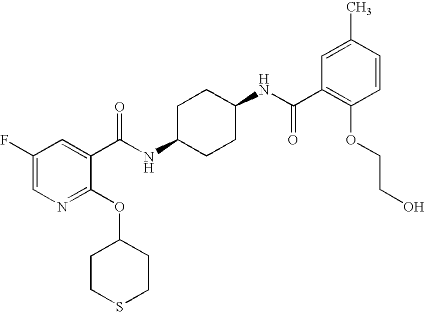
or a pharmaceutically acceptable salt or solvate thereof.

12. A pharmaceutical composition comprising a compound of claim 1, or a pharmaceutically acceptable salt or solvate thereof, and a pharmaceutically acceptable excipient, diluent or carrier.

13.-19. (canceled)

20. A process for preparing a compound of claim 1, comprising reacting a compound of formula (VI), 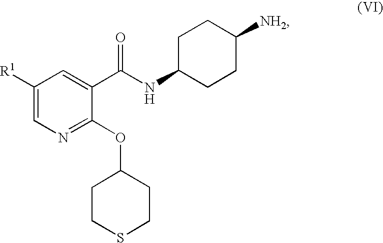
with a reagent of formula Y-Z-R².
wherein
R¹ is H, halo or (C₁-C₄)alkyl;
Z is CO or SO₂;
R² is phenyl, benzyl, naphthyl, heteroaryl or (C₃-C₈)cycloalkyl;
said phenyl, benzyl, naphthyl, heteroraryl and (C₃-C₈)cycloalkyl each being substituted with one of (C₁-C₆)alkoxy ((C₃-C₈)cycloalkyl)-(C₁-C₆)alkoxy, hydroxy(C₂-C₆)alkoxy, ((C₃-C₈)cycloalkyl)oxy or phenyl substituted by (C₁-C₆)alkoxy;
said phenyl, benzyl, naphthyl, heteroraryl and (C₃-C₈)cycloalkyl are each additionally optionally substituted independently with one or two halo; CN; CONR³R⁴; (C₁₋₆ alkyl optionally substituted with one to three halo; OH; hydroxy(C₁-C₆)alkyl: ((C₁-C₈)cycloalkyl(C₁-C₆)alkyl: (C₃-C₈)cycloalkyl; or NR³R⁴; and
R³ and R⁴ are each independently H, (C₁-C₄)alkyl or SO₂ (C₁-C₄)alkyl,
and Y is a leaving group.

21. A process for preparing a compound of claim 1, comprising reacting a compound of formula (IX) 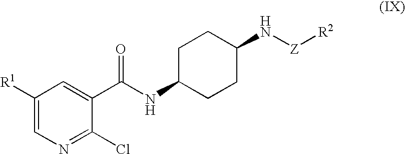
with tetrahydrothiopyran-4-ol.

22. A process for preparing a compound of claim 1, comprising reacting a compound of formula (XII) with a compound of formula (VIII):

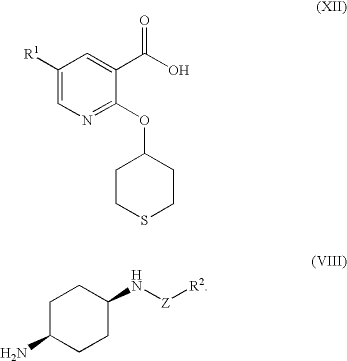

23. A compound of formula (IX): 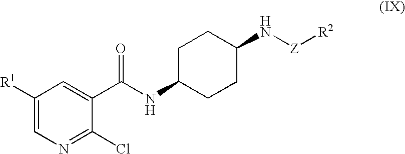
wherein
R¹ is H, halo or (C₁-C₄)alkyl:
Z is CO or SO₂;
R² is phenyl, benzyl, naphthyl, heteroaryl or (C₃-C₈)cycloalkyl;
said phenyl, benzyl, naphthyl, heteroraryl and (C₃-C₈)cycloalkyl each being substituted with one of (C₁-C₆)alkoxy, ((C₃-C₈)cycloalkyl)-(C₁-C₆)alkoxy, hydroxy(C₂-C₆)alkoxy, ((C₃-C₈)cycloalkyl)oxy and phenyl substituted by (C₁-C₆)alkoxy;
said phenyl, benzyl, naphthyl, heteroraryl and (C₃-C₈)cycloalkyl are each additionally optionally substituted independently with one or two halo; cyano; CONR³R⁴: (C₁-C₆)alkyl optionally substituted with one to three halo; hydroxy; hydroxy(C₁-C₆)alkyl;
((C₃-C₈)cycloalkyl)-(C₁-C₆)alkyl; (C₃-C₈)cycloalkyl; or NR³R⁴; and
R³ and R⁴ are each independently H, (C₁-C₄)alkyl or SO₂(C₁-C₄)alkyl.

24. A combination comprising a compound of claim 1 with another therapeutic agent selected from:
(a) 5-Lipoxygenase (5-LO) inhibitors or 5-lipoxygenase activating protein (FLAP) antagonists,
(b) Leukotriene antagonists (LTRAs) including antagonists of LTB4, LTC4, LTD4, and LTE4,
(c) Histaminic receptor antagonists including H1, H3 and H4 antagonists,
(d) α1- and α2-adrenoceptor agonist vasoconstrictor sympathomimetic agents for decongestant use,
(e) Muscarinic M3 receptor antagonists or anticholinergic agents,
(f) β2-adrenoceptor agonists,
(g) Theophylline,
(h) Sodium cromoglycate,
(i) COX-1 inhibitors (NSAIDs) and COX-2 selective inhibitors,
(j) Oral or inhaled Glucocorticosteroids,
(k) Monoclonal antibodies active against endogenous inflammatory entities,
(I) Anti-tumor necrosis factor (anti-TNF-a) agents,
(m) Adhesion molecule inhibitors including VLA-4 antagonists,
(n) Kinin-B1- and B2-receptor antagonists,
(O) Immunosuppressive agents,
(p), Inhibitors of matrix metalloproteases (MMPs),
(q) Tachykinin NK1, NK2 and NK3 receptor antagonists,
(r) Elastase inhibitors,
(s) Adenosine A2a receptor agonists,
(t) Inhibitors of urokinase,
(u) Compounds that act on dopamine receptors, e.g. D2 agonists,
(v) Modulators of the NFkb pathway, e.g. IKK inhibitors,
(w) Agents that can be classed as mucolytics or anti-tussive,
(x) antibiotics, and
(y) p38 MAP kinase inhibitors.

25. A method of treating a disease, disorder or condition in which PDE4 inhibition is beneficial in a mammal suffering from a disease, disorder or condition in which PDE4 inhibition is beneficial, said method comprising administering to said mammal in need of such treatment a therapeutically effective amount of a compound of claim 1, a pharmaceutically acceptable salt thereof or a pharmaceutical composition comprising a compound of claim 1 or a pharmaceutically acceptable salt thereof and a pharmaceutically acceptable carrier, diluent or excipient.

26. A method of claim 25 wherein the disease, disorder or condition is selected from:
asthma of whatever type, etiology, or pathogenesis, in particular asthma that is a member selected from the group consisting of atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, non-atopic asthma, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma and wheezy infant syndrome,
chronic or acute bronchoconstriction, chronic bronchitis, small airways obstruction, and emphysema,
obstructive or inflammatory airways diseases of whatever type, etiology, or pathogenesis, in particular an obstructive or inflammatory airways disease that is a member selected from the group consisting of chronic eosinophilic pneumonia, chronic obstructive pulmonary disease (COPD), COPD that includes chronic bronchitis, pulmonary emphysema or dyspnea associated therewith, COPD that is characterized by irreversible, progressive airways obstruction, adult respiratory distress syndrome (ARDS) and exacerbation of airways hyper-reactivity consequent to other drug therapy
pneumoconiosis of whatever type, etiology, or pathogenesis, in particular pneumoconiosis that is a member selected from the group consisting of aluminosis or bauxite workers' disease, anthracosis or miners' asthma, asbestosis or steam-fitters' asthma, chalicosis or flint disease, ptilosis caused by inhaling the dust from ostrich feathers, siderosis caused by the inhalation of iron particles, silicosis or grinders' disease, byssinosis or cotton-dust asthma and talc pneumoconiosis;
bronchitis of whatever type, etiology, or pathogenesis, in particular bronchitis that is a member selected from the group consisting of acute bronchitis, acute laryngotracheal bronchitis, arachidic bronchitis, catarrhal bronchitis, croupus bronchitis, dry bronchitis, infectious asthmatic bronchitis, productive bronchitis, staphylococcus or streptococcal bronchitis and vesicular bronchitis,
bronchiectasis of whatever type, etiology, or pathogenesis, in particular bronchiectasis that is a member selected from the group consisting of cylindric bronchiectasis, sacculated bronchiectasis, fusiform bronchiectasis, capillary bronchiectasis, cystic bronchiectasis, dry bronchiectasis and follicular bronchiectasis,
seasonal allergic rhinitis or perennial allergic rhinitis or sinusitis of whatever type, etiology, or pathogenesis, in particular sinusitis that is a member selected from the group consisting of purulent or nonpurulent sinusitis, acute or chronic sinusitis and ethmoid, frontal, maxillary, or sphenoid sinusitis,
rheumatoid arthritis of whatever type, etiology, or pathogenesis, in particular rheumatoid arthritis that is a member selected from the group consisting of acute arthritis, acute gouty arthritis, chronic inflammatory arthritis, degenerative arthritis, infectious arthritis, Lyme arthritis, proliferative arthritis, psoriatic arthritis and vertebral arthritis,
gout, and fever and pain associated with inflammation,
an eosinophil-related disorder of whatever type, etiology, or pathogenesis, in particular an eosinophil-related disorder that is a member selected from the group consisting of eosinophilia, pulmonary infiltration eosinophilia, Loffler's syndrome, chronic eosinophilic pneumonia, tropical pulmonary eosinophilia, bronchopneumonic aspergillosis, aspergilloma, granulomas containing eosinophils, allergic granulomatous angiitis or Churg-Strauss syndrome, polyarteritis nodosa (PAN) and systemic necrotizing vasculitis,
atopic dermatitis, allergic dermatitis, contact dermatitis, or allergic or atopic eczema,
urticaria of whatever type, etiology, or pathogenesis, in particular urticaria that is a member selected from the group consisting of immune-mediated urticaria, complement-mediated urticaria, urticariogenic material-induced urticaria, physical agent-induced urticaria, stress-induced urticaria, idiopathic urticaria, acute urticaria, chronic urticaria, angioedema, cholinergic urticaria, cold urticaria in the autosomal dominant form or in the acquired form, contact urticaria, giant urticaria and papular urticaria,
conjunctivitis of whatever type, etiology, or pathogenesis, in particular conjunctivitis that is a member selected from the group consisting of actinic conjunctivitis, acute catarrhal conjunctivitis, acute contagious conjunctivitis, allergic conjunctivitis, atopic conjunctivitis, chronic catarrhal conjunctivitis, purulent conjunctivitis and vernal conjunctivitis,
uveitis of whatever type, etiology, or pathogenesis, in particular uveitis that is a member selected from the group consisting of inflammation of all or part of the uvea, anterior uveitis, iritis, cyclitis, iridocyclitis, granulomatous uveitis, nongranulomatous uveitis, phacoantigenic uveitis, posterior uveitis, choroiditis; and chorioretinitis,
psoriasis;
multiple sclerosis of whatever type, etiology, or pathogenesis, in particular multiple sclerosis that is a member selected from the group consisting of primary progressive multiple sclerosis and relapsing remitting multiple sclerosis,
autoimmune/inflammatory diseases of whatever type, etiology, or pathogenesis, in particular an autoimmune/inflammatory disease that is a member selected from the group consisting of autoimmune hematological disorders, hemolytic anemia, aplastic anemia, pure red cell anemia, idiopathic thrombocytopenic purpura, systemic lupus erythematosus, polychondritis, scleroderma, Wegner's granulomatosis, dermatomyositis, chronic active hepatitis, myasthenia gravis, Stevens-Johnson syndrome, idiopathic sprue, autoimmune inflammatory bowel diseases, ulcerative colitis, endocrin opthamopathy, Grave's disease, sarcoidosis, alveolitis, chronic hypersensitivity pneumonitis, primary biliary cirrhosis, juvenile diabetes or diabetes mellitus type I, keratoconjunctivitis sicca, epidemic keratoconjunctivitis, diffuse interstitial pulmonary fibrosis or interstitial lung fibrosis, idiopathic pulmonary fibrosis, cystic fibrosis, glomerulonephritis with and without nephrotic syndrome, acute glomerulonephritis, idiopathic nephrotic syndrome, minimal change nephropathy, inflammatory/hyperproliferative skin diseases, benign familial pemphigus, pemphigus erythematosus, pemphigus foliaceus, and pemphigus vulgaris,
prevention of allogeneic graft rejection following organ transplantation,
inflammatory bowel disease (IBD) of whatever type, etiology, or pathogenesis, in particular inflammatory bowel disease that is a member selected from the group consisting of collagenous colitis, colitis polyposa, transmural colitis, ulcerative colitis and Crohn's disease (CD),
septic shock of whatever type, etiology, or pathogenesis, in particular septic shock that is a member selected from the group consisting of renal failure, acute renal failure, cachexia, malarial cachexia, hypophysial cachexia, uremic cachexia, cardiac cachexia, cachexia suprarenalis or Addison's disease, cancerous cachexia and cachexia as a consequence of infection by the human immunodeficiency virus (HIV),
liver injury,
pulmonary hypertension of whatever type, etiology or pathogenesis including primary pulmonary hypertension/essential hypertension, pulmonary hypertension secondary to congestive heart failure, pulmonary hypertension secondary to chronic obstructive pulmonary disease, pulmonary venous hypertension, pulmonary arterial hypertension and hypoxia-induced pulmonary hypertension,
bone loss diseases, primary osteoporosis and secondary osteoporosis,
central nervous system disorders of whatever type, etiology, or pathogenesis, in particular a central nervous system disorder that is a member selected from the group consisting of depression, Alzheimers disease, Parkinson's disease, learning and memory impairment, tardive dyskinesia, drug dependence, arteriosclerotic dementia and dementias that accompany Huntington's chorea, Wilson's disease, paralysis agitans, and thalamic atrophies,
infection, especially infection by viruses wherein such viruses increase the production of TNF-α in their host, or wherein such viruses are sensitive to upregulation of TNF-α in their host so that their replication or other vital activities are adversely impacted, including a virus which is a member selected from the group consisting of HIV-1, HIV-2, and HIV-3, cytomegalovirus (CMV), influenza, adenoviruses and Herpes viruses including Herpes zoster and Herpes simplex,
yeast and fungus infections wherein said yeast and fungi are sensitive to upregulation by TNF-α or elicit TNF-α production in their host, e.g., fungal meningitis, particularly when administered in conjunction with other drugs of choice for the treatment of systemic yeast and fungus infections, including but are not limited to, polymixins, e.g. Polymycin B, imidazoles, e.g. clotrimazole, econazole, miconazole, and ketoconazole, triazoles, e.g. fluconazole and itranazole as well as amphotericins, e.g. Amphotericin B and liposomal Amphotericin B,
ischemia-reperfusion injury, ischemic heart disease, autoimmune diabetes, retinal autoimmunity, chronic lymphocytic leukemia, HIV infections, lupus erythematosus, kidney and ureter disease, urogenital and gastrointestinal disorders and prostate diseases,
reduction of scar formation in the human or animal body, such as scar formation in the healing of acute wounds, and
psoriasis, other dermatological and cosmetic uses, including antiphlogistic, skin-softening, skin elasticity and moisture-increasing activities.

27. A method of claim 26 wherein the disease, disorder or condition is chronic obstructive pulmonary disease, asthma or chronic bronchitis.
